(19) United States
(12) Patent Application Publication
(10) Pub. No.: US 2008/0124400 A1
(43) Pub. Date: May 29, 2008

(54) Microparticles With High Loadings Of A Bioactive Agent

(75) Inventors: Richard T. Liggins, Coquitlam (CA); Philip M. Toleikis, Vancouver (CA); Dechi Guan, Vancouver (CA)

Correspondence Address: SEED INTELLECTUAL PROPERTY LAW GROUP PLLC, 701 FIFTH AVENUE, SUITE 5400, SEATTLE, WA, 98104-7092, US

(73) Assignee: Angiotech International AG, Zug (CH)

(21) Appl. No.: 11/630,819

(22) PCT Filed: Jun. 24, 2005
(86) PCT No.: PCT/US05/22443

§ 371 Date: Jan. 28, 2008

Related U.S. Application Data
(60) Provisional application No. 60/582,610, filed on Jun. 24, 2004.

(51) Int. Cl. A61K 31/337; A61K 9/14

(52) U.S. Cl. 424/501; 514/449

(57) ABSTRACT

The present invention discloses compositions, devices and methods for the production, use and administration of compositions that comprise microparticles that are loaded with a drug at a concentration of greater than 50% (weight drug/weight microparticle).

### TECHNICAL FIELD

The present invention relates generally to pharmaceutical compositions and methods for the production and use of the compositions, which include microparticles having a high loading of a drug, that is, greater than 50% weight drug/weight microparticle.

### BACKGROUND

Microparticles for use as drug delivery systems have been the focus of development and optimization for several decades because they offer the potential of injectable controlled release of bioactive agents. Generally, about 0.0001 to 30% by weight of a drug can be loaded into a microparticle, with 0.001 to 20% being most common (see, e.g., WO 03/005961; Bain et al., J. Microencapsul. 1999(16), 369-85; Vachon et al., J. Microencapsul. 1995(12), 287-305; Knepp et al., J. Pharm. Pharmacol. 1993(45), 887-91; Fournier et al., Cancer 2003(97) 2822-29; Boisdron-Celle et al., J. Pharm. Pharmacol. 1995(47), 108-14; Ramtoola et al.; J. Microencapsul. 1992(9), 415-23; He et al., Acta Pharmacol. Sin. 2001(22), 530-3; and O'Hara et al., Pharm Res. 2000(17), 955-61). Paclitaxel loaded microspheres which have been reported in the literature include, e.g., U.S. Pat. Nos. 6,515,016, 6,333,347, 6,537,585, 6,350,464, 6,419,709, 6,395,300, 6,447,796, 6,277,391, 6,200,547, and 5,626,862, Burt et al., Cancer Letters 1995(88) 73-9; Attawia et al., J Control Release 2001(71) 193-202; Wang et al., Chem Pharm Bull 1996(44) 1935-40; Das et al., J Biomed Mater Res 2001(55) 96-103; Chandy et al., Drug Delivery 2001(8) 77-86; Mu and Feng, J Control Release 2001(76) 239-54; Dordunoo et al., Cancer Chemother Pharmacol 1995(36) 279-82; Harper et al., Clin Cancer Res 1999(5) 4242-8; Mu and Fen, J. Control. Rel 2003(86) 33-48; Demetrick et al., Am J Surg 1997(173) 403-6; and Liggins et al., Biomaterials 2000(21) 1959-69; Int J Pharm 2001(222) 19-33. Higher loadings (e.g., up to 50% w/w) have been reported for certain drugs (see, e.g., Polakovic M. et al., J Control Release 1999(60), 169-77; Rajaonarivony et al., J Pharm Sci. 1993(82), 912-7; Owusu-Ababio et al., J Microencapsul 1996(13), 195-205; Hariharan et al., J Microencapsul 2002(19), 95-109; Li et al., Pharm Res. 1994(11), 1792-9; Spenlehauer et al., J Pharm Sci. 1986(75), 750-5; Zhang et al., Yao Xue Xue Bao. 1994(29), 544-9; Bodmeier et al., J Pharm Sci. 1993(82), 191-4; Thanoo et al., Passerini et al., J Pharm Pharmacol 2002(54), 913-9; Ozsoy et al., Boll Chim Farm 2002(141), 29-32; Bunjes et al., Pharm Res. 2001(18), 287-93; Kim et al′; Biomaterials 2001(22), 2049-56; Gorner et al., J. Control. Rel. 1999(57), 259-68; Karasulu et al., Eur. J. Pharm. Sci. 2003(19) 99-104; Uzunkaya and Bergisadi, Farmaco. 2003(58) 509-12; Yamada et al., J. Control. Rel. 2001(75) 271-82; and U.S. Pat. No. 6,447,796). Microspheres having drug loadings in excess of 50% w/w; however, have been reported for only a few compounds (see, e.g., Bodmeier et al., J Microencapsul 1992(9), 89-98; Shukla et al., Pharm Res. 1991(8), 1396-400; Hejazi et al., Int J Pharm 2002(235), 87-94; Owusu-Ababio et al., J Control Release 1999(57), 151-9; Al-Maaieh et al., J Control Release 2001(70), 169-81; Curley et al., Anesthesiology 1996(84) 1401-10; Wong et al., J. Control. Rel. 2002(84) 99-114; and U.S. Pat. No. 6,515,016).

### SUMMARY OF INVENTION

Briefly stated, the present invention provides compositions that comprise microspheres having a high loading (i.e., higher than 50% w/w) of one or more bioactive agents useful in treating a variety of medical conditions. The bioactive agent (i.e., drug) contained in the microparticles may be selected from a variety of therapeutically active compounds for which sustained or local release may provide a benefit to the patient. The present compositions may be administered to a patient (e.g., a mammal, such as a human) in need thereof to effectively treat or prevent various medical conditions, such as, but not limited to, cancer, benign fibrotic hyperplasia, vascular diseases, surgical adhesions, inflammatory conditions, psoriasis, restenosis, arthritis, infection, pain, and aneurysms.

High loading compositions may facilitate less frequent dosing and may exhibit increased efficacy, or altered pharmacokinetics, distribution or metabolism in the body, and decreased toxicity or side effects relative to equivalent doses given in a different, conventional formulation. Furthermore, the microspheres described herein may be prepared using less excipient than for standard microsphere compositions, resulting in improved degradation, biocompatibility and drug release from the composition.

The drug-loaded compositions of the present invention may have one or more of the following features: sufficiently biocompatible, with drug release profiles desirable for a given clinical application, not prone to aggregation, without undesirable entrapment of drug that would result in too slow or incomplete release, and reasonably safe and well tolerated.

In one aspect, the present invention provides compositions comprising a microparticle wherein the microparticle comprises a polymer and a drug, and wherein the drug is present in the microparticle at a concentration of greater than 50% (weight of drug/weight of microparticle). In certain embodiments, the drug may be present in the microparticle at a concentration of greater than 55%, 60%, 65%, 70%, 75%, 80%, 85%, 90%, or 95% (weight of drug/weight of microparticle).

In certain embodiments, the polymer may be or comprise a synthetic polymer, such as a polyester or polyether. In certain embodiments, the synthetic polymer may be or comprise a polyester that contains the residues of one or more of the monomers selected from lactide, lactic acid, glycolide, glycolic acid, ε-caprolactone, γ-caprolactone, hydroxyvaleric acid, hydroxybutyric acid, β-butyrolactone, γ-butyrolactone, gamma-valerolactone, γ-decanolactone, δ-decanolactone, trimethylene carbonate, 1,4-dioxane-2-one and 1,5-dioxepan-2one. The polyester may further include a residue having a chemical formula [—OC₆H₄COOH]. Exemplary polyesters include, but are not limited to, poly(L-lactide) (PLLA), poly(DL-lactide) (PDLLA), lactide copolymers, poly(glycolide), poly(DL-lactide-co-glycolide) (PLGA), poly(ε-caprolactone), poly(δ-decanolactone), poly(δ-valerolactone), or poly(lactic acid) (PLA). In other embodiments, the polymer is a polyether, such as a polyether that includes a residue of polyethylene glycol (PEG) or a copolymer thereof (e.g., PLA-block-PEG, PLGA-block-PEG, and polypropylene oxide-block-PEG).

In certain other embodiments, the polymer may be or comprise a biologically derived polymer, such as a polysaccharide (e.g., chitosan, cellulose, alginate, and derivatives thereof).

In certain embodiments, the polymer is bioresorbable. In certain other embodiments, the polymer is non-biodegradable such as poly(methylmethacrylate), poly(styrene), and poly(divinylbenzene).

Each of the polymeric microparticles may be used to load each of the drugs disclosed herein. For instance, in certain embodiments, an anti-cancer agent (e.g., paclitaxel, cisplatin, 5-fluorouracil, doxorubicin, mitoxantrone, etoposide, and derivatives and analogues thereof) may be combined with various polymers disclosed herein (e.g., polyesters, polylactide, lactide copolymers, and polylacgtide-co-glycolide).

In certain other embodiments, the drug is an anti-fibrotic agent (e.g., paclitaxel, mitomycin C, 5-fluorouracil, an interferon, D-penicillamine, β-aminoproprionitrile, and analogues and derivatives thereof).

In certain other embodiments, the drug is an anti-infective agent (e.g., an antibiotic including cephalexin, rifampicin, griseofulvin, tetracycline, ciprofloxacin, erythromycin, silver-containing organic compounds, and analogues and derivatives thereof).

In certain other embodiments, the drug is an anti-inflammatory agent (e.g., aspirin, hydrocortisone, naproxen, indomethacin, ketoprofen, and analogues and derivatives thereof).

In certain other embodiments, the drug is a neurologically active agent (e.g., pentoxyfyline, fluphenazine, bupivicaine, lidocaine, naltrexone, and analogues and derivatives thereof).

In certain other embodiments, the drug is an anti-restenotic agent (e.g., paclitaxel, sirolimus, tacrolimus, everolimus, analogues and derivatives thereof).

In certain other embodiments, the drug is an anti-oxidant agent.

In certain other embodiments, the drug is a fibrosing agent.

In certain embodiments, the drug is an anti-microtubule agent (e.g., a taxane, including paclitaxel and analogues and derivatives thereof).

In one aspect, the microparticles of the present invention may be in the form of a microsphere. The microparticles and microspheres of the invention may have an average diameter of between about 0.5 μm and about 1000 μm, between about 0.5 μm and about 500 μm, between about 0.5 μm to about 200 μm, between about 0.5 μm and about 100 μm, between about 0.5 μm and about 50 μm, between about 0.5 μm and about 25 μm, between about 0.5 μm and about 10 μm, or between about 1 μm and about 10 μm.

In certain embodiments, the compositions may further include a carrier. The carrier may be in the form of a gel, hydrogel, paste, ointment, cream, tablet, capsule, spray, powder, film, or surgical sealant.

In certain embodiments, the described compositions may further include a scaffold. The scaffold may be a medical device, such as, packing material, gauze, stents, screws, pins, plates, artificial joints, sutures, catheters, grafts, stent-grafts, shunts, spinal implants, artifical discs, aneurysm coils, heart valves, and implantable brachytherapy devices. The scaffold may be a porous matrix (e.g., fabrics, meshes, porous films, sponges, and pledgets). The scaffold may include a polymer, e.g., polyethylene, silicone, ethylene vinyl acetate copolymer, polyethylene terephthalate, fluorinated polyethylene derivatives, and polyurethane.

In certain embodiments, the microsphere further comprises a stabilizer, including polymeric stabilizers (e.g., poly(vinyl alcohol), dextran sulfate, polyvinylpyrollidone, carbopol, and polaxamer 188).

In certain embodiments, the drug is paclitaxel or an analogue or a derivative thereof, and the polymer is selected from the group consisting of polyesters, polylactide, lactide copolymers, and polylactide-co-glycolide.

In certain other embodiments, the drug is lidocaine or an analogue or a derivative thereof, and the polymer is selected from the group consisting of polyesters, polylactide, lactide copolymers, and polylactide-co-glycolide.

Also provided by the present invention are processes for making the compositions of the instant invention. In one aspect, a method for producing a polyester is provided, comprising polymerizing a composition comprising one or more of the monomers selected from the group consisting of lactide, lactic acid, glycolide, glycolic acid, ε-caprolactone, γ-caprolactone, hydroxyvaleric acid, hydroxybutyric acid, β-butyrolactone, gamma-butyrolactone, gamma-valerolactone, γ-decanolactone, δ-decanolactone, trimethylene carbonate, 1,4-dioxane-2-one and 1,5-dioxepan-2-one using a polymerization initiator, wherein the polymerization initiator is salicylic acid.

In one aspect, a method for manufacturing a medical device is provided that comprises combining a scaffold and a microparticle, wherein the microparticles comprises a polymer and a drug, wherein the drug is present in the microparticle at a concentration of greater than 50% (weight of drug/weight of microparticle). In certain embodiments, the drug is present in the microparticle at a concentration of greater than 55%, 60%, 65%, 70%, 75%, 80%, 85%, 90%, or 95% (weight of drug/weight of microparticle).

In another aspect, a method for making a microparticle is provided that comprises combining a polymer and a drug, such that the drug is present in the microparticle at a concentration of greater than 50%, 55%, 60%, 65%, 70%, 75%, 80%, 85%, 90%, or 95% (weight of drug/weight of microparticle).

In yet another aspect, kits are provided that include (i) a container containing microparticles with high loadings of a drug and (ii) another container containing a carrier. In certain embodiments, the drug is a local anesthetic, such as lidocaine. In certain embodiments, the carrier is a tissue filler (such as a collagen composition). The kits may further comprise (iii) a device for combining the microparticles and the carrier.

In a related aspect, kits are provided that include (i) a container containing microparticles with high loadings of a drug and (ii) a scaffold.

In another aspect, there is provided by the instant invention a method for treating a disease or condition, comprising administering to a patient in need thereof a therapeutically effective amount of a composition comprising microparticles having a high loading of an indicated drug as described herein. In a further aspect, the method comprises delivering the therapeutic composition to a target site or confined space within the body.

Compositions of the present invention may be administered by a variety of routes, depending on the condition targeted for treatment. In certain embodiments, the route of administration comprises intraarticular, intraperitoneal, topical, intravenous, intramuscular, subcutaneous, ocular, oral, rectal, into the urinary/genital tract, or to a surgically incised area, such as resection margins, incision, and anastomosis.

In one aspect, a method of treating an inflammatory condition is provided that includes administering to a patient in need thereof an effective amount of a composition in accordance with the invention, wherein the drug is an anti-inflammatory agent, an analgesic, anti-neoplastic agent, anti-restenotic agent, anti-infective agent, hemostatic agent, or an anti-microtubule agent (e.g., paclitaxel and analogues and derivatives thereof).

In another aspect, a method of treating an infection is described that includes administering to a patient in need thereof an effective amount of a composition in accordance with the invention, wherein the drug is an antibiotic or an anti-infective agent (e.g., penicillin, cephalosporin, erythromycin, and quinolone).

In yet another aspect, a method of treating a neoplastic disease is described that includes administering to a patient in need thereof an effective amount of a composition in accordance with the invention, wherein the drug is an anti-neoplastic agent (such as paclitaxel and analogues and derivatives thereof).

In another aspect, a method of treating fibrosis is described that comprises administering to a patient in need thereof an effective amount of a composition in accordance with the invention, wherein the drug is an anti-fibrotic agent (such as paclitaxel and an analogue or a derivative thereof).

In another aspect, a method of regenerating tissue is described that comprises administering to a patient in need thereof an effective amount of a composition in accordance with the invention, wherein the drug is heparin, an analogue thereof, or a growth factor.

In another aspect, a method for tissue filling is provided that comprises administering to a patient in need thereof an effective amount of a composition in accordance with the invention. In certain embodiments, the drug is a local anesthetic (such as lidocaine). In certain embodiments, the composition may further comprise a polymeric carrier, such as collagen.

In another aspect, a method for treating restenosis is provided that comprises administering to a patient in need thereof an effective amount of a composition in accordance with the invention wherein the drug is an anti-restenotic agent (such as paclitaxel and analogues and derivatives thereof).

These and other aspects of the present invention will become evident upon reference to the following detailed description and attached drawings. In addition, various references are set forth herein which describe in more detail certain procedures, devices, or compositions, and are therefore incorporated by reference in their entirety.

### BRIEF DESCRIPTION OF THE DRAWINGS

FIG. 1 is a graph showing cumulative release (%) over the course of 15 days for 40%, 70%, and 90% (w/w) paclitaxel loaded PLLA microspheres.

FIG. 2 is graph showing cumulative release (%) over the course of 15 days for 70% (w/w) paclitaxel in 1200, 2000, and 45,000 MW PLLA.

FIG. 3 is a graph showing the change in paclitaxel content of microparticles by weight (% w/w) over a period of three weeks for samples dissolved in water.

### DETAILED DESCRIPTION

Prior to setting forth the invention, it may be helpful to an understanding thereof to set forth definitions of certain terms that will be used hereinafter.

The terms “active agent,” “bioactive agent,” “biologically active agent,” “therapeutic agent,” “pharmacologically active agent,” and “drug” are used interchangeably herein to refer to a chemical material or compound suitable for administration to a patient and that induces a desired effect. The terms include agents that are therapeutically effective as well as prophylactically effective. Also included are derivatives and analogs of those compounds or classes of compounds specifically mentioned that also induce the desired effect.

“Microparticle” as used herein refers to a particle with a diameter (i.e., the distance spanning the widest point, or points, of the microparticle) of about 0.5 μm to 1000 μm. Microparticles may have regular or irregular shapes.

“Microsphere” as used herein refers to a microparticle that is essentially spherical in shape. Microspheres may be spherical, elliptoid or have a shape that approximates such a spherical or elliptoid shape, and may be smooth or have disruptions such as cracks or dimples. Microspheres typically have a mean diameter between about 0.5 μm and about 1000 μm.

In certain embodiments, the microparticles or microspheres have a preferred average diameter of at least about 0.5 μm, 1 μm, 5 μm, 10 μm, 20 μm, 50 μm or 100 μm, the optimal size being determined by the desired drug release properties and the application. In certain embodiments, the microparticles have a preferred average diameter of no more than about 5 μm, 10 μm, 20 μm, 50 μm, 100 μm, 150 μm, 250 μm, 500 μm, or 1000 μm, the optimal size being determined by the desired drug release properties and the application.

“Theoretical loading” as used herein refers to the amount of drug incorporated into the microparticle expressed in terms of the mass percent drug in the microparticle (w/w %), where the remaining mass is accounted for by the presence of at least one excipient. The number is determined by the ratio of drug to excipients charged in the manufacturing process. Depending on the method of preparation, and on typical variation within the method, the drug and excipient(s) will be incorporated into the microparticles with characteristic efficiencies. Unless the efficiencies of incorporation of the drug and all other components are equal, and no significant impurities or residuals are present, the theoretical loading level will not be the exact amount of drug in the microparticles. Despite this inequality, the theoretical loading level is still a useful measure since in provides a value related to the actual loading based on the encapsulation efficiency.

“Measured loading” as used herein refers to the amount of drug incorporated into a microparticle on a % w/w basis. The number is determined by measurement or inference (described below) of the actual amount of drug contained in the microparticle irrespective of the theoretical loading. The measured loading may be determined analytically or inferred by a number of means known to those skilled in the art. Measurement may be quantitative, for instance based on a comparison of drug levels relative to that in a known reference standard. Alternately, measurement may be semi-quantitative, as in a limit test. Any suitable analytical method may be employed, such as, compendial methods described in the current (or other stated) edition of the United States Pharmacopeia, or any other method demonstrated to be suitable for measurement of the drug within the microparticles. Suitable instrumentation useful for measuring drug loading is dependent on the drug to be measured, including for various embodiments of the invention, spectroscopic instruments (e.g., infrared, fluorescence, and ultraviolet spectrographs), titrators (for titratable drugs such as acids and bases) and substrate based assays such as ELISA. Substrate based assays which measure drug content in terms of activity should be used also to determine the inherent activity of the drug so that the activity-based loading measurement may be related to the mass of drug in microparticles. Alternatively, the measured loading may be determined by inference. Total content may be inferred, for example, by determining the solubility concentration of a given drug in the continuous phase used in forming microspheres by the solvent evaporation method wherein an organic drug-polymer solution is suspended in an immiscible, usually aqueous continuous phase. Assuming that drug may be transferred from the organic to the continuous phase, not more drug than can be dissolved in that phase may be transferred without crystallization of the drug. By knowing the saturation solubility and the volume of the continuous phase and provided that no crystals are observed to have formed (determined microscopically), the minimum inferred (measured) total content may be taken as the total initially loaded minus the mass of drug which can be dissolved in the continuous phase.

“Encapsulation efficiency” as used herein refers to the ratio of measured loading to theoretical loading, expressed as a percentage. A number less than 100% indicates that less drug was encapsulated into the microparticles per gram of excipient than was charged in the manufacturing process.

“Carrier” as used herein refers to a substance that facilitates the delivery of microparticles according to the present invention or a composition comprising the microparticles. It may be in a liquid, semi-solid, or solid form. In certain embodiments, the carrier is mixed with microparticles or a composition that comprises microparticles before administration into a patient. In certain other embodiments, the carrier and microparticles or a composition that comprises microparticles may be mixed at the site of administration. In certain embodiments, the carrier is a polymeric carrier. In certain embodiments, a carrier facilitates the delivery of microparticles by forming an injectable or syringable mixture with the microparticles, by providing a vehicle suitable for delivery to a specific administration site, or by allowing sustained and/or controlled release of a drug present in the composition. In certain embodiments, the carrier may be a solid or semi-solid substrate with exterior and/or interior surface(s) onto which microparticles or a composition comprising microparticles may be applied. Such a substrate is also referred to as “scaffold.”

The term “stabilizer” refers to a compound that is present in a microparticle and stabilizes the microparticle. In certain embodiments, the stabilizer is polymeric.

“Anti-inflammatory agent” should be understood to include any polypeptide, or molecule that impairs the inflammatory process in either cell culture or in vivo. A wide variety of methods may be utilized to determine the anti-inflammatory activity of a particular compound including, for example, assays described by Tak et al. in Mechanisms of Inflammation, Section 2 of Firestein et al., (eds.)

“Polysaccharide” refers to a combination of at least three monosaccharides that are generally joined by glycosidic bonds. Naturally occurring polysaccharides may be purified according to accepted procedures known to those having skill in the art at the time of this invention. Polysaccharides may be ionically or chemically cross-linked by groups such as vinyl sulfone (see U.S. Pat. No. 4,605,691) or other polymers of low molecular weight (see U.S. Pat. No. 4,582,865). One class of polysaccharides is cellulose polymers, which are polymers of glucose units of which a defined proportion may be derivatized, for example, with methyl or acetate groups.

“Polypeptide” includes peptides, proteins, cyclic proteins, branched proteins, polyamino acids, copolymers thereof, and derivatives of each of these (including those with non-naturally occurring amino acids known in the art), which may be naturally or synthetically derived.

“Fibrosis,” “scarring,” or “fibrotic response” refers to the formation of fibrous tissue in response to injury or medical intervention.

Therapeutic agents which inhibit fibrosis or scarring are referred to herein as “anti-fibrotic agents,” “fibrosis-inhibiting agents,” “anti-scarring agents,” and the like, where these agents inhibit fibrosis through one or more mechanisms including: inhibiting angiogenesis, inhibiting migration or proliferation of connective tissue cells (such as fibroblasts, smooth muscle cells, vascular smooth muscle cells), reducing ECM production, and/or inhibiting tissue remodeling.

“Inhibit fibrosis,” “reduce fibrosis,” and the like are used synonymously to refer to the action of agents or compositions which result in a statistically significant decrease in the formation of fibrous tissue that can be expected to occur in the absence of the agent or composition.

Therapeutic agents which promote (also referred to interchangeably herein as induce, stimulate, cause, increase, accelerate, and the like) fibrosis or scarring are referred to interchangeably herein as “fibrosis-inducing agents,” “scarring agents,” “fibrosing agents,” “adhesion-inducing agents,” and the like. These agents promote fibrosis through one or more mechanisms including, for example, inducing or promoting angiogenesis, stimulating migration or proliferation of connective tissue cells (such as fibroblasts, smooth muscle cells, vascular smooth muscle cells), inducing extracellular matrix (ECM) production, and promoting tissue remodeling. In addition, numerous therapeutic agents described herein can have the additional benefit of promoting tissue regeneration (the replacement of injured cells by cells of the same type).

“Host,” “person,” “subject,” “patient” and the like are used synonymously to refer to the living being into which the compositions provided herein are administered.

“Inhibitor” refers to an agent that prevents a biological process from occurring or slows the rate or degree of occurrence of a biological process. The process may be a general one such as scarring or refer to a specific biological action such as, for example, a molecular process resulting in release of a cytokine.

“Anti-microtubule agents” should be understood to include any protein, peptide, chemical, or another molecule that impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al., (Cancer Lett 79(2):213-219, 1994) and Mooberry et al., (Cancer Lett. 96(2):261-266, 1995).

“Medical device,” “implant,” “medical device or implant,” “implant/device” and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing, replacing or augmenting damaged or diseased organs and tissues.

“Bioresorbable” as used herein refers to the property of a composition or material being able to be cleared from a body after administration to a human or animal. Bioresorption may occur by one or more of a variety of means, such as, for example, dissolution, oxidative degradation, hydrolytic degradation, enzymatic degradation, metabolism, clearance of a component, its breakdown product, or its metabolite through routes such as, for example, the kidney, intestinal tract, lung or skin.

“Biodegradable” refers to materials for which the degradation process is at least partially mediated by, and/or performed in, a biological system. “Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or pronation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds that simultaneously contain a basic group and an acidic group, for example a carboxyl group in addition to basic nitrogen atoms, can be present as zwifterions. Salts can be obtained by customary methods known to those skilled in the art, for example by combining a compound with an inorganic or organic acid or base in a solvent or diluent, or from other salts by cation exchange or anion exchange.

A “tissue filler” refers to a composition that is implanted into a tissue to increase the volume of the tissue for cosmetic purposes or for treating disorders associated with an improperly reduced tissue volume. A tissue filler is generally biocompatible (i.e., substantially non-toxic), non-allergenic (i.e., produce no or tolerable levels of immune and inflammatory responses), and durable (i.e., present at the site of administration for at least one month). It may be biodegradable or partially biodegradable.

The term “effective amount” refers to the amount of an agent or composition that provides the effect desired. The actual amount that is determined to be an effective amount will vary depending on factors such as the size, general health and condition, sex and age of the patient and can be more readily determined by the caregiver.

“Concentration by weight (w/w)” refers to the ratio in percentage of the weight of a drug to that of a microparticle in which the drug is present.

Concentration Ranges: Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. As used herein, the term “about” means±15%.

As used herein, the terms “average” or “mean” include the arithmetic mean as well as any appropriate weighted averages such as are used in the expression of polymeric molecular weight or particle size distributions.

### Microparticle Compositions

In one aspect, the present application provides microparticles and compositions that comprise microparticles. The microparticle comprises a drug and a polymer, where the drug is at a concentration of greater than 50% (w/w). The microparticle may comprise additional components such as a stabilizer. The composition that comprises the microparticles may also comprise additional components such as a carrier, including a scaffold.

A. Therapeutic Agents

Microparticles of the present invention may include a wide variety of therapeutic agents (used interchangeably with “drugs”). In certain embodiments of the invention, the drugs may be selected from a variety of therapeutically active compounds for which sustained release may provide a benefit to the patient.

Representative examples of classes of therapeutic agents (which are efficacious in one of a number of indications) include, for example, vitamins, anti-infectives, anti-inflammatories, anticancer agents, immunosuppressants, antihistamines, antipsychotics, antiangiogenic compounds, analgesics, diuretics, lipid or cholesterol lowering agents, anticoagulants, anticonvulsants, anti-thrombotic agents, profibrotic agents, anti-fibrotic agents, fibrosing agents, vasoconstrictors, vasodilators, antiarhythmics, narcotics, narcotic antagonists, antibiotics, retinols, sedatives, stimulants, thyroid stimulants, thyroid hormone suppressants, labor inducing agents, sunscreens, blood glucose level modifying compounds, or neuromuscular blockers or relaxants. In certain embodiments, the therapeutic agent has at least one of anti-inflammatory, antibiotic, anti-infective, anti-microtubule, anti-fibrotic, fibrosis-inducing, antioxidant, anti-restontic, anticancer activity, and neurological or anaesthetic activities.

The present compositions may include any number of hydrophobic and/or hydrophilic drugs, and the drug may be water-soluble or water-insoluble. For example, compositions are described that include a drug with a water solubility at 25° C. of less than 10% (weight of drug/volume of water), less than 2% (w/v), less than 1% (w/v), or less than 0.75% (w/v), less than 0.5% (w/v), or less than 0.1% (w/v) as measured by techniques such as quantitative chromatography, and spectroscopic methods such as UV or IR absorption.

Microparticles may be loaded with drugs having any molecular weight. In certain embodiments, microparticles are described which include a drug having a molecular weight of greater than 445 g/mol (e.g., paclitaxel, rapamycin, geldanamycin and its analogues, etoposide, vancomycin, vincristine and its analogues). In certain embodiments, the compound has at least 23 carbon atoms (e.g., paclitaxel, angiotensisn, polymyxin, oxytocin, docetaxel, codeine, irinotecan, vitamins E and D, cephalosporines, buprinorphine, loperamide, raloxifene, beclomethasone, hydrocortisone, interferons, somatotropins, and certain bioactive peptides). In certain embodiments, microparticles are described which include 50% (w/w) or greater of a drug having a molecular weight of less than 180 g/mol (e.g., pyrimidine derivatives such as 5-fluorouracil, phenol derivatives such as silver fluoride (MW=127), phenylpropanolamine (MW=151), nicotinic acid (MW=123), flucytrosine (MW=129), tryptamine (MW=160), salicylic acid (sodium salt) (MW=160) and fenadiazole (MW=162)).

In certain embodiments (such as forming microparticles with certain polymers and with certain drug concentration), the drug is not (i) ibuprofen (when wax, paraffin, or semi-synthetic glyceryl esters are the polymer and the drug concentration is 60% or lower), (ii) theophyline (when cellulose acetate is the polymer and the drug concentration is 60% or lower), (iii) tetracycline (when chitosan is the polymer and the drug concentration is 69% or lower), (iv) ciprofloxacin (when PLLA is the polymer and the drug concentration is 71% or lower), (v) bupivicaine (when polylactic-co-glycolic acid is the polymer and the drug concentration is 75% or lower), (vi) sulfathiazole (when chitosan is the polymer and the drug concentration is 60% or lower), or (vii) paclitaxel (when nylon is the polymer and the drug concentration is 60% or lower).

1. Fibrosing Agents

In certain embodiments, the drug may be an agent that promotes fibrosis or scarring. Therapeutic agents that promote fibrosis or scarring can do so through one or more mechanisms including: inducing or promoting angiogenesis, stimulating migration or proliferation of connective tissue cells (such as fibroblasts, smooth muscle cells, vascular smooth muscle cells), inducing ECM production, and/or promoting tissue remodeling. In addition, numerous therapeutic agents described in this invention will have the additional benefit of also promoting tissue regeneration (the replacement of injured cells by cells of the same type). Fibrosis-inducing agents are described, e.g., in the U.S. patent application entitled “Medical Implants and Fibrosis-Inducing Agents,” filed Nov. 20, 2004 (U.S. Ser. No. 10/986,230) and in the U.S. patent application entitled “Compositins and Methods for Treating Diverticular Disease,” filed May 12, 2005 (U.S. Ser. No. 11/129,763), both applications are incorporated by reference in their entireties. Examplary fibrosing agents include, but are not limited to, silk (such as silkworm silk, spider silk, recombinant silk, raw silk, hydrolyzed silk, acid-treated silk, and acylated silk), talc, chitosan, polylysine, fibronectin, bleomycin or an analogue or derivative thereof, a fibrosing agent that connective tissue growth factor (CTGF), metallic beryllium or an oxide thereof, copper, saracin, silica, crystalline silicates, quartz dust, talcum powder, ethanol, a component of extracellular matrix, collagen, fibrin, fibrinogen, poly(ethylene terephthalate), poly(ethylene-co-vinylacetate), N-carboxybutylchitosan, an RGD protein, a polymer of vinyl chloride, cyanoacrylate, crosslinked poly(ethylene glycol)-methylated collagen, an inflammatory cytokine, TGFβ, PDGF, VEGF, TNFα, NGF, GM-CSF, IGF-a, IL-1, IL-8, IL-6, a growth hormone, a bone morphogenic protein, a cell proliferative agent, dexamethasone, isotretinoin, 17-β-estradiol, estradiol, diethylstibesterol, cyclosporine a, all-trans retinoic acid or an analogue or derivative thereof, wool (including animal wool, wood wool, and mineral wool), cotton, bFGF, polyurethane, polytetrafluoroethylene, poly(alkylcyanoacrylate), activin, angiopoletin, insulin-like growth factor (IGF), hepatocyte growth factor (HGF), a colony-stimulating factor (CSF), erythropoietin, an interferon, endothelin-1, angiotensin II, bromocriptine, methylsergide, fibrosin, fibrin, an adhesive glycoprotein, proteoglycan, hyaluronan, secreted protein acidic and rich in cysteine (SPaRC), a thrombospondin, tenacin, a cell adhesion molecule, an inhibitor of matrix metalloproteinase, a tissue inhibitor of matrix metalloproteinase, methotrexate, carbon tetrachloride, and thioacetamide.

2. Anti-Fibrotic Agents

In certain embodiments, the drug may be an agent that inhibits fibrosis or scarring. Therapeutic agents which inhibit fibrosis or scarring can do so through one or more mechanisms including: inhibiting angiogenesis, inhibiting migration or proliferation of connective tissue cells (such as fibroblasts, smooth muscle cells, vascular smooth muscle cells), reducing ECM production, and/or inhibiting tissue remodeling. In addition, numerous therapeutic agents described in this invention will have the additional benefit of also reducing tissue regeneration (the replacement of injured cells by cells of the same type) when appropriate. Fibrosis-inhibiting agents are described, e.g., in U.S. patent application, “Medical Implants and Anti-Scarring Agents,” filed Nov. 10, 2004 (U.S. Ser. No. 10/986,231); and “Anti-Scarring Agents, Therapeutic Compositions, and Use Thereof,” filed May 10, 2005 (U.S. Ser. No. 60/679,293). Exemplary anti-fibrotic agents include, but are not limited to, cell cycle inhibitors (e.g., doxorubicin, mitoxantrone, TAXOTERE, vinblastine, tubercidin, paclitaxel, and analogues and derivatives thereof), podophyllotoxins (e.g., etoposide), immunomodulators (e.g., sirolimus and everolimus), heat shock protein 90 antagonists (e.g., geldanamycin) and analogues and derivatives thereof, HMGCOA reductase inhibitors (e.g., simvastatin) and analogues and derivatives thereof, inosine monophosphate dehydrogenase inhibitors (e.g., mycophenolic acid, 1-alpha-25 dihydroxy vitamin D₃) and analogues and derivatives thereof, NF kappa B inhibitors (e.g., Bay 11-7082) and analogues and derivatives thereof, antimycotic agents (e.g., sulconizole) and analogues and derivatives thereof, p38 MAP kinase inhibitors (e.g., SB202190) and analogues and derivatives thereof, and anti-angiogenic agents (e.g., halofuginone bromide) and analogues and derivatives. Additional exemplary anti-fibrotic agents include, but are not limited to, ZD-6474 (an angiogenesis inhibitor), AP-23573 (an mTOR inhibitor), synthadotin (a tubulin antagonist), S-0885 (a collagenase inhibitor), aplidine (an elongation factor-1 alpha inhibitor), ixabepilone (an epithilone), IDN-5390 (an angiogenesis inhibitor and an FGF inhibitor), SB-2723005 (an angiogenesis inhibitor), ABT-518 (an angiogenesis inhibitor), combretastatin (an angiogenesis inhibitor), anecortave acetate (an angiogenesis inhibitor), SB-715992 (a kinesin antagonist), temsirolimus (an mTOR inhibitor), adalimumab (a TNFα antagonist), erucylphosphocholine (an ATK inhibitor), alphastatin (an angiogenesis inhibitor), BXT-51072 (an NF Kappa B inhibitor), etanercept (a TNFα antagonist and TACE inhibitor), humicade (a TNFα inhibitor), and gefitinib (a tyrosine kinase inhibitor), as well as analogues and derivatives of the aforementioned.

3. Anti-Inflammatory Agents and Analgesics

In certain embodiments, the drug to be incorporated into microparticles of the present invention may have anti-inflammatory activity or analgesic activity. In these embodiments, the drug may be selected from a non-steroidal anti-inflammatory agent (including aspirin, ibuprofen, indomethacin, naproxen, prioxicam, diclofenac, tolmetin, fenoclofenac, meclofenamate, mefenamic acid, etodolac, sulindac, carprofen, fenbufen, fenoprofen, flurbiprofen, ketoprofen, oxaprozin, tiaprofenic acid, phenylbutazone diflunisal, salsalte, and salts and analogues thereof); opiates (including codeine, meperidine, methadone, morphine, pentazocine, fentanyl, hydromorphone, oxycodone, oxymorphone, and salts and analogues thereof); steroidal antiinflammatories including hydrocortisone and esters thereof. In one embodiment, the drug incorporated may be an anti-inflammatory agent such as naproxen or indomethacin. In yet other embodiments, the anti-inflammatory agent is ketoprofen or an analogue or derivative thereof.

Exemplary compositions of the present invention that include anti-inflammatory agents include, without limitation, polylactide, lactide copolymer, polyester, or poly(glycolide-co-lactide) microspheres having at least 60% w/w aspirin, 60% w/w indomethacin, 70% w/w ibuprofen, 70% w/w naproxen, or 70% w/w hydrocortisone.

4. Antibiotic and Anti-Infective Agents

In certain embodiments, the bioactive agent may be an antibiotic or anti-infective agent, which may act by a number of mechanisms. They may be anthelmintics (including mebendazole, niclosamide, piperazine, praziquante, thibendazole and pyrantel pamoate); aminoglycosides (including tobramycin, gentamicin, amikacin and kanamycin); antifungals (including amphotericin B, clotrimazole, fluconazole, ketoconazole, itraconazole, miconazole, nystatin, and griseofulvin); cephalosporins (including cefazolin, cefotaxime, cefoxitin, defuroxime, cefaclor, cefonicid, cefotetan, cefoperazone, ceftriaxone, cephalexin, moxalactam, and ceftazidime, and salts thereof); β-lactams (including aztreonam, and imipenem); chloramphenicol and salts thereof; erythromycins and salts thereof (including roxithromycin, erythromycin, and its esters such as ethylsuccinate, guceptate and stearate); penicillins (including penicillin G, amoxicillin, amdinocillin, ampicillin, carbenicillin, ticarcillin, cloxacillin, nafcillin, penicillin V, and their salts and esters); tetracyclines (including tetracycline, and doxycycline, and salts thereof); clindamycin; polymixin B; vancomycin; ethambutol; isoniazid; rifampin; rifampicin; antivirals (including acyclovir, zidovudine, vidarabine); anti-HIV drugs; quinolones (including ciprofloxacin); sulfonamides; nitrofurantoin; metronidazole; clofazimine; triclosan and chlorhexidine. Antibiotic agents also include active analogues and derivatives of the aforementioned antibiotic agents. In certain embodiments, the antibiotic of the invention has additional therapeutic activities as anticancer and/or anti-restenotic activities.

In certain embodiments, the drug incorporated may be an antibiotic such as a sulfonamide. For example, sulfathiazole may be loaded into polylactide, lactide copolymer, polyester, or polylactide-co-glycolide microparticles at a level of higher than about 65% (e.g., about 70%, 75%, 80%, 85%, 90%, or 95%).

Additional exemplary compositions within the scope of the invention that include anti-infective agents are, without limitation, polylactide, lactide copolymer, polyester, or poly(glycolide-co-lactide) microspheres having at least (w/w) 50% cephalexin, 50% rifampicin, 60% griseofulvin, 75% tetracycline, or 75% ciprofloxacin, or 70% erythromycin, or 50% of a silver organic compound or salt, or silver chloride. These exemplary compositions may further include a scaffold such as a suture, catheter, or orthopedic device.

5. Anti-Microtubule Agents

A wide variety of anti-microtubule agents can be utilized in the present invention to form high drug loading microparticles. Representative examples of anti-microtubule agents include taxanes, colchicine, LY290181, glycine ethyl ester, aluminum fluoride, and CI 980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), vinca alkaloids (e.g., vinblastine and vincristine), discodermolide (ter Haar et al., Biochemistry 35: 243-250, 1996), as well as analogues and derivatives of any of these (see also PCT/CA97/00910 (WO 98/24427), which as noted above is hereby incorporated by reference in its entirety, for a list of additional anti-microtubule agents).

Within one embodiment of the invention, the anti-microtubule agent is paclitaxel, a compound that disrupts mitosis (M-phase) by binding to tubulin to form abnormal mitotic spindles, or an analogue or derivative thereof.

The utility of the anti-microtubule agent paclitaxel, as a component of the compositions that comprise part of this invention, is demonstrated by data from a series of in vitro and in vivo experiments. Paclitaxel inhibits neutrophil activation (Jackson et al., Immunol. 90:502-10, 1997), decreases T-cell response to stimuli, and inhibits T-cell function (Cao et al., J. Neuroimmunol. 108:103-11, 2000), prevents the proliferation of and induces apoptosis in synoviocytes (Hui et al., Arth. Rheum. 40:1073-84, 1997), inhibits AP-1 transcription activity via reduced AP-1 binding to DNA (Hui et al., Arth. Rheum. 41:869-76, 1998), inhibits collagen induced arthritis in an animal model (Brahn et al., Arth. Rheum. 37:839-45, 1994; Oliver et al., Cellular Immunol. 157:291-9, 1994) but is non-toxic to non-proliferating cells, such as normal chondrocytes and non-proliferating synoviocytes (Hui et al., Arth. Rheum. 40:1073-84, 1997).

Paclitaxel, formulations, prod rugs, epimers, isomers, analogues and derivatives thereof may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol, phosphonoxy and carbonate derivatives of taxol, taxol 2′,7-di(sodium 1,2-benzenedicarboxylate, 10-desacetoxy-11,12-dihydrotaxol-10,12(18)-diene derivatives, prodrugs including 2′-and/or 7-O-ester, amide, thioester derivatives, (2′-and/or 7-O-carbonate derivatives), fluoro taxols, 9-deoxotaxol, 7-deoxy-9-deoxotaxol, 10-desacetoxy-7-deoxy-9-deoxotaxol, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′-succinyltaxol; 2′-(beta-alanyl)-taxol); 2′-γ-aminobutyryltaxol formate; ethylene glycol derivatives of 2′-succinyltaxol; prodrugs or derivatives having amino acids attached at either or both of the 2′ and 7 positions (R₉ and R₃, respectively); 2′-glutaryltaxol; 2′-(N,N-dimethylglycyl) taxol; 2′-(2-(N,N-dimethylamino)propionyl)taxol; 2′-orthocarboxybenzoyl taxol; 2′-aliphatic carboxylic acid derivatives of taxol, prodrugs {2′-(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, TAXOL (Bristol-Myers Squibb Company, New York, N.Y.) analogues with modified phenylisoserine side chains, taxotere, (N-debenzoyl-N-tert-(butoxycaronyl)-1,0-deacetyltaxol, cephalomannine, Taxol C, Taxol D, Taxol E, Taxol F, brevifoliol, yunantaxusin and taxusin, debenzoyl-2-acyl paclitaxel derivatives, benzoate paclitaxel derivatives, sulfonated 2′-acryloyltaxol; sulfonated 2′-O-acyl acid paclitaxel derivatives, C18-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, C7 taxane derivatives, C10 taxane derivatives, 2-debenzoyl and 2-acyl paclitaxel derivatives, taxane analogues bearing new C2 and C4 functional groups, n-acyl paclitaxel analogues, 10-deacetyl taxol B, and 10-deacetyl taxol, benzoate derivatives of taxol, 2-aroyl-4-acyl paclitaxel analogues, ortho-ester paclitaxel analogues, and deoxy paclitaxel and deoxy paclitaxel analogues.

In one aspect, the anti-microtubule agent is a taxane having the formula (C1):

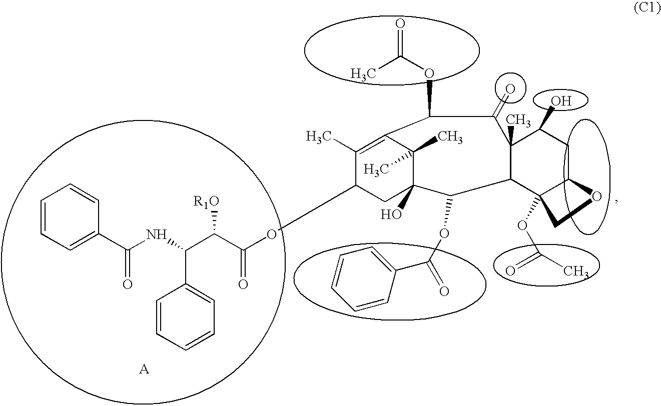

where the gray-highlighted portions may be substituted and the non-highlighted portion is the taxane core. A side-chain (labeled “A” in the diagram) is desirably present in order for the compound to have good activity as an anti-microtubule agent. Examples of compounds having this structure include paclitaxel (Merck Index entry 7117), docetaxol (TAXOTERE, Merck Index entry 3458, Aventis Pharma S. A., France), and 3′-desphenyl-3′-(4-nitrophenyl)-N-debenzoyl-N-(t-butoxycarbonyl)-10-deacetyltaxol.

In certain embodiments, suitable taxanes such as paclitaxel and its analogues and derivatives are disclosed in U.S. Pat. No. 5,440,056 as having the structure (C2):

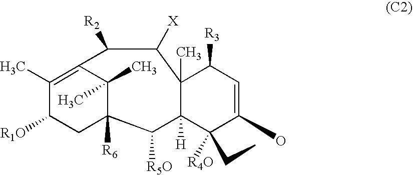

wherein X may be oxygen (paclitaxel), hydrogen (9-deoxotaxol or 9-deoxy derivatives, which may be further substituted to yield taxanes such as 7-deoxy-9-deoxotaxol, 10-desacetoxy-7-deoxy-9-deoxotaxol,), thioacyl, or dihydroxyl precursors; R₁ is selected from paclitaxel or taxotere side chains or an alkanoyl of the formula (C3)

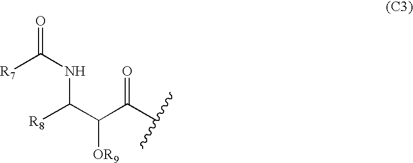

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy to yield taxanes that include in some cases with further substitution: 10-deacetyltaxol, 10-desacetoxy-11,12-dihydrotaxol-10,12(18)-diene derivatives, 10-deacetyl taxol A, 10-deacetyl taxol B; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In certain embodiments, the paclitaxel analogues and derivatives useful as anti-microtubule agents in the present invention are disclosed in PCT International Patent Application No. WO 93/10076. As disclosed in this publication, the analogue or derivative should have a side chain attached to the taxane nucleus at C₁₃, as shown in the structure below (formula C4), in order to confer antitumor activity to the taxane.

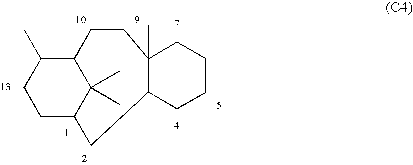

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based anti-microtubule agent useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

In one embodiment, the anti-microtubule agent is a taxane (e.g., paclitaxel or an analogue or derivative thereof). Exemplary compositions that comprise anti-microtubule agents include, but are not limited to, polyester, polylactide, lactide copolymer, or polylactide-co-glycolide microparticles containing greater than 50% w/w, or greater than 60%, or greater than 70%, or greater than 80%, or greater than 90% of paclitaxel or a derivative or analogue thereof.

6. Cardiovascular and Anti-Restenotic Agents

In certain embodiments, therapeutic drugs may be agents that inhibit some or all of the processes involved in the development of intimal hyperplasia, such as cell proliferation, cell migration and matrix deposition. Agents in this category include cell cycle inhibitors and/or anti-angiogenic agents (e.g., anthracyclines and taxanes), immunosuppressive compounds (e.g., sirolimus and its analogues and derivatives), and non-steroidal anti-inflammatory agents (e.g., dexamethasone and its analogues and derivatives). Furthermore, antithrombotic agents and antiplatelet agents may also be loaded into polymeric microparticles.

In certain embodiments, the therapeutic agent is sirolimus, or a derivative or an analogue thereof. Sirolimus (also referred to as “rapamycin”) is a macrolide antibiotic. Sirolimus analogues useful in the present invention include tracolimus and derivatives thereof (e.g., EP0184162B1 and U.S. Pat. No. 6,258,823), and everolimus and derivatives thereof (e.g., U.S. Pat. No. 5,665,772). Further representative examples of sirolimus analogues and derivatives include ABT-578 and others can be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507, 5,985,890, 5,604,234, 5,597,715, 5,583,139, 5,563,172, 5,561,228, 5,561,137, 5,541,193, 5,541,189, 5,534,632, 5,527,907, 5,484,799, 5,457,194, 5,457,182, 5,362,735, 5,324,644, 5,318,895, 5,310,903, 5,310,901, 5,258,389, 5,252,732, 5,247,076, 5,225,403, 5,221,625, 5,210,030, 5,208,241, 5,200,411, 5,198,421, 5,147,877, 5,140,018, 5,116,756, 5,109,112, 5,093,338, and 5,091,389.

7. Anticancer Agents

Anticancer agents suitable to be incorporated into microparticles of the present invention may act by a number of mechanisms. These agents may be antimetabolites, anti-microtubule agents, chelating agents, antibiotics or antiangiogenic agents. Exemplary anticancer agents useful in the present invention include, but are not limited to, alkylating agents such as bis(chloroethyl)amines (including cyclophosphamide, mechlorethamine, chlorambucil, or melphalan), nitrosoureas (including carmustine, estramustine, lomustine or semustine), aziridines (including thiotepa or triethylenemelamine), alkylsulfonates including busulfan, other agents with possible alkylating agent activity (including procarbazine, cisplatin, carboplatin, dacarbazine, or hexamethylmelamine); antimetabolites such as methotrexate, mercaptopurine, thioguanine, 5-fluorouracil, cytarabine, azacitidine; plant alkaloids such as vinca alkaloids (including vincristine, vinorelbine, or vinblastine), bleomycin, dactinomycin, anthracyclines (including daunorubicin or doxorubicin, idarubicin, epirubicin, pirarubicin, zorubicin carubicin, anthramycin, mitoxantrone, menogaril, nogalamycin, aclacinomycin A, olivomycin A, chromomycin A₃, and plicamycin), etoposide, teniposide, mithramycin, mitomycin; hormonal agents such as androgens (including testosterone, or fluoxymestrone), antiandrogens including flutamide, estrogens (including diethylstilbesterol, estradiol, ethylestradiol, or estrogen), antiestrogens including tamoxifen, progestins (including hydroxyprogesterone, progesterone, medroxyprogesterone, or megestrol acetate), adrenocorticosteroids (including hydrocortisone, or prednisone), gonadotropin-releasing hormones and agonists thereof including leuprolide; cytadrenl other anticancer agents (including amscarine, asparaginase, hydroxyurea, mitotane, quniacrine); and anti-microtubule agents including paclitaxel and docetaxol. Also included are analogues and derivatives of the aforementioned compounds. Additional anticancer agents may be defined as compounds which exhibit therapeutic activity against cancer, as defined using standard tests known in the art, including in vitro cell studies, in vivo and ex vivo animal studies and clinical human studies. Suitable tests are described in texts such as “Anticancer Drug Development Guide” (B. A. Teicher ed., Humana Press, 1997 Totowa, N.J.). Other anticancer agents include antiangiogenic agents such as active taxanes as described above, including paclitaxel and docetaxol; angiostatic steroids including squaline; cartilage derived proteins and factors; thrombospondin; matrix metalloproteinases (including collagenases, gelatinases A and B, stromelysins 1, 2 and 3, martilysin, metalloelastase, MT1-MMP (a progelatenase), MT2-MMP, MT3-MMP, MT4-MMP, Bay 12-9566 (Bayer), AG-3340 (Agouron), CGS27023! (Novartis), Chiroscience compounds D5140, D1927, D2163); and phytocemicals (including genistein, daidzein, leuteolin, apigenin, 3 hydroxyflavone, 2′,3′-dihydroxyflavone, 3′,4′-dihydroxyflavone, or fisetin). Anti-angiogentic agents also include active analogues and derivatives of the aforementioned antiangiogenic agents. Certain anticancer agents are also classified as antifibrotic agents. These include mitomycin C, 5-fluorouracil, interferons, D-penicillamine and β-aminoproprionitrile.

Exemplary compositions within the scope of the invention that include polyester, polylactide, lactide copolymer, polylatide-co-glycolide microparticles that comprise at least 50% w/w cisplatin, 50% w/w 5-fluorouracil, 50% w/w doxorubicin, 55% w/w mitoxantrone, or 50% w/w etoposide.

8. Neurologically Active Agents

In certain embodiments of the invention, the drug incorporated into microparticles in a high loading is neurologically active. Such drugs may have the following therapeutic activities: anticonvulsants, antipsychotics, anaesthetics and antidepressants, anti-Parkinson's disease compounds, and anti-Alzheimer's disease compounds. Exemplary anticonvulsants include barbiturates (such as secobarbital, phenobarbital, amobarbital and primidone); benzodiazepines such as clonazepam; hydantoins such as phenyloin; succinimides such as ethosuximide, and valproic acid. Exemplary antidepressants include tricyclic antidepressants such as amitriptylline, desipramine, doxepin, imipramine, nortriptylline, protriptyline, and trimipramine; heterocyclics such as maprotiline, nefazodone, venlafaxine, amoxapine, trazodone, alprazolam, and fluoxetine and chlropropiophenones such as bupropion; and serotonin reuptake inhibitors such as fluoxetine, fluvoxamine, and paroxetine. Antipsychotic agents include haloperidol, loxapine, molindone, perphenazine, thioridazine, trifluoperazine, thiotixene, chlorpromazine, and fluphenazine. Exemplary anaesthetics include methohexital sodium, thiopental sodium, etomidate, keatmine, propofol, bupivicaine, chloroprocaine, etidocaine, lidocaine, mepivicaine, prilocalne, procaine, tetracaine, benzocaine, cocaine, dibucainem dyclonnine, and pramoxine. Exemplary anti-Parkinson's disease compounds include selegiline (L-deprenyl). Salts (for example hydrochlorides and sodium salts), esters, prodrugs, analogues and derivatives of the aforementioned compounds are additional exemplary neurologically active agents.

Other drugs useful in the present invention include immunomodulatory agents such as cyclosporine A and mycophenolic acid, including analogues, ester prodrugs and derivatives thereof; drugs useful in treating certain lung disorders, such as theophylline or pentoxyffyline. The drug incorporated in the microsphere may also be an aesthetic such as lidocaine, xylocalne, etidocaine, carobicaine, xylocalne, marcaine, nesacaine, etiod, or bupivicaine. For example, microparticles are described containing about 40% (e.g., lidocaine) to greater than about 80% (e.g., bupivicaine).

Exemplary compositions within the scope of the invention that comprise neurologically active agents are, without limitation, polyester, polylactide, lactide copolymer, or poly(glycolide-co-lactide) microspheres having at least 50% (w/w) lidocaine, at least 60% w/w cyclosporine A, 65% w/w theophylline, 60% pentoxyfyline, 50% fluphenazine, 80% bupivicaine, or 50% naltrexone.

In certain embodiments, microparticles may be prepared that include a combination (e.g., a blend) of two or more of the aforementioned bioactive agents.

9. Antioxidant Agents

Antioxidant agents suitable to be incorporated into microparticles of the present invention may act by a number of mechanisms. They may be vitamins (e.g., vitamins C and E) or quinolone compounds (e.g., BHA and BHT), amino acids (e.g., N-acetylcysteine), a metal or metal containing molecule or salt having an antioxidant metal such as selenium, cadmium, zinc or vanadium, particularly metals with a +2 valence, other compounds such as repaglinide, carnosine, antioxidant extracts or fractions thereof from green or black teas, alpha-lipoic acid, or antioxidant enzymes. Particularly suitable antioxidants include hydrophobic molecules having a melting point above 40° C., including analogs and derivatives of the aforementioned antioxidants.

B. Polymers

In addition to a drug, the microparticles of the present invention also comprise a polymer. The term “polymer,” as used herein, refers to a macromolecule formed by the chemical union of five or more identical monomers. In the case of hydrocarbon monomers, greater than about 80 units are required. Generally, hydrocarbon structures comprising fewer of hydrocarbon monomers (e.g., —CH₂— groups) are waxes, particularly when the structure comprises an ester linkage in the linear chain structure, for example, beeswax which comprises hydrocarbon chains of C₃₆-ester-C₃₆.

Suitable polymers include biologically derived as well as synthetic materials. For example, biologically derived polymers such as hyaluronic acid and derivatives thereof, dextran and derivatives thereof, cellulose and derivatives thereof (e.g., methylcellulose, hydroxypropylcellulose, hydroxypropylmethylcellulose, carboxymethylcellulose, cellulose acetate phthalate, cellulose acetate propionate, cellulose acetate succinate, cellulose acetate butyrate, hydroxypropylmethylcellulose phthalate), chitosan and derivatives thereof, β-glucan, arabinoxylans, carrageenans, pectin, glycogen, fucoidan, chondrotin, pentosan, keratan, alginate, cyclodextrins, and salts and derivatives, including esters and sulphates thereof, may be used in the present invention. In further embodiments, the biologically derived polymer may be a polypeptide such as poly(L-glutamic acid), collagen, albumin, fibrin and gelatin.

In yet other embodiments, the polymeric excipient may be synthetic. Synthetic polymer include, for example, homopolymers, copolymers or cross-linked polymers comprising polyethers such as polyethylene glycol, polyesters such as poly(lactide)s and poly(lactic acid)s, which include L- and D-isomers as well as mixtures of D and L in any ratio, such as 50:50 (DL), poly(glycolide), copolymers of poly(glycolide) and poly(lactide)s (PLGA), polycaprolactones namely poly(ε-caprolactone) (PCL), or polyvalerolactones, such as poly(γ-valerolactone), polymers of acrylic acid and derivatives thereof, such as polyacrylic acid or polymethylmethacrylate, polyurethanes, polyethylene, polyethylene glycol, polystyrene, ethylene vinyl acetate, poloxamers, silicones, polystyrene, polypropylene, crosslinked divinyl benzene, vinyls such as polyvinyl chloride, polyvinyl acetate, and polyvinyl alcohol, polythioesters, polyanhydrides, polyamides (e.g., nylon), and polyorthoesters.

Polymers may be linear, branched, block, graft, random or alternating copolymers, and may be crosslinked either chemically or ionically.

The polymers may be a biodegradable or a bioresorbable polymer (e.g., poly(lactide), poly(lactic acid), poly(glycolide), copolymers of poly(glycolide) and poly(lactide)s (PLGA), polycaprolactones) or a non-biodegradable polymer (e.g., poly(methylmethacrylate), poly(styrene), and poly(divinylbenzene)).

Polymers for use in preparing compositions having a high percentage of drug loading may have any molecular weight depending on the type of polymer and the desired application. In certain embodiments, the composition includes a polymer having a relatively low average molecular weight (e.g., a weight average molecular weight (M_{w}) of less than 100,000 g/mol or a number average molecular weight (Mₙ) of 67,000 g/mol or less, as measured by GPC). Polymers may have weight average molecular weights, for example, of less than about 75,000 g/mol, or less than about 50,000 g/mol, or less than about 25,000 g/mol, or less than about 10,000 g/mol, or less than 5000 g/mol.

Representative examples of polymers that can be synthesized with M_{w} falling below 100,000 include polyesters such as poly(lactic acid) (e.g., PLLA), poly(caprolactone), and PLGA.

In certain embodiments, a microsphere may having a high loading of drug, such as 50% w/w or higher and include a polymer having a relativetly low molecular weight (i.e., Mₙ less than 67,000; M_{w} less than 100,000). In some embodiments, the loading may be 60% w/w or more. In yet other embodiments, for certain drugs, the loading may be 75% or 80% or 90% w/w or more.

In other embodiments, the polymer may have a molecular weight of greater than 100,000 (e.g., polysaccharides, such as chitosan, alginates, and certain types of synthetic polymers, such as polyesters and polyethylene, polystyrene, polymethylmethacrylates, polyethylene oxide, multiblock polymers of polyoxyethylene and polyoxypropylene). In certain embodiments, a microsphere may having a high loading of drug, such as 50% w/w or higher and include a polymer having a relativetly high molecular weight (i.e., Mₙ=67,000 or greater or M_{w}=100,000 or greater). In some embodiments, the loading may be 60% w/w or more. In yet other embodiments, for certain drugs, the loading may be 75% or 80% or 90% w/w or more.

Also suitable are derivatives and combinations (i.e., blends and copolymers) of the aforementioned polymers. Derivatization may be accomplished by the inclusion of unique end groups, pendant groups, or monomeric units within the backbone, which may be spaced randomly, regularly or with a defined density. These may include acids, bases, ionizing species, complexing species, halogens, latent degradation sites, such as thio- or phosphoesters, hydrophobic groups such as phenyl containing groups, or groups with latent functionality for example cross-linkers such as succinimides.

C. Carriers

In certain embodiments of the present invention, a composition that contains high drug loaded microparticles may further comprise a carrier. The microparticles may be dispersed throughout the carrier or may be contained in only certain regions of the carrier, for example, being contained inside a capsule. The carrier may be a solid or a liquid. Carriers themselves or in combination with microparticles may include or form, for example, gels, hydrogels, suspension mediums, capsules, tablets, powders, inserts (e.g., vaginal inserts), suppositories, pastes, creams, sprays, ointments, films, sealants, and scaffolds. In certain embodiments, the carrier provides for delivery of the drug loaded microparticles or facilitates administration of the microparticles. In some embodiments, microparticles are suspended in a gel or other liquid having in it suspending agents. In some other embodiments, microparticles may be dispersed in a cream, ointment, tablet, suppository, or vaginal insert having other excipients typically found in such formulations. In yet other embodiments, microparticles or compostions comprising microparticles may be combined with various scaffolds. Carriers useful in the present invention may be prepared according to methods well known to those skilled in the art, including those described in texts such as Remington's Pharmaceutical Sciences, 17^{th} edition (A. Gennaro ed., Mack Publishing Company, 1986 Easton Pa.).

1. Gels and Hydrogels

A gel is a clear or translucent and uniform colloidal mixture of a soft and malleable consistency in a more solid form than a solution. It consists of a solid component dissolved in a dispersion medium. A gel may be a hydrogel in which the dispersion medium is primarily water. Alternatively, a gel may be anorganogel in which the dispersion medium is primarily a non-aqueous fluid.

In certain embodiments, gels possess properties such as elevated viscosity and elasticity, which may be reduced with increased dilution with an aqueous medium such as water. In certain other embodiments, gels may maintain an elevated level of viscosity and elasticity when diluted with an aqueous solution, such as water.

In certain embodiments, gels may contain only non-crosslinked and/or partially crosslinked polymers. Alternately, gel may contain only crosslinked polymers (see, e.g., Goodell et al., Am. J. Hosp. Pharm. 43:1454-1461, 1986; Langer et al., “Controlled release of macromolecules from polymers”, in Biomedical Polymers, Polymeric Materials and Pharmaceuticals for Biomedical Use, Goldberg, E. P., Nakagim, A. (eds.) Academic Press, pp. 113-137,1980; Rhine et al., J. Pharm. Sci. 69:265-270, 1980; Brown et al., J. Pharm. Sci. 72:1181-1185, 1983; and Bawa et al., J. Controlled Release 1:259-267, 1985). Crosslinking may be accomplished by several means including covalent, hydrogen, ionic, hydrophobic, chelation complexation, and the like.

In certain embodiments of the instant invention, the carrier gel may include a polypeptide or polysaccharide. In some aspects, the polysaccharides and polypeptides of the instant invention can be fashioned to exhibit a variety of forms with desired release characteristics and/or with specific desired properties. For example, polymers can be formed into gels by dispersing them into a solvent such as water. In certain embodiments, polysaccharides and polypeptides and other polymers can be fashioned to release microparticles and/or a therapeutic agent present in the microparticles upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polysaccharides include carboxymethyl cellulose, cellulose acetate trimellilate, hydroxypropylmethylcellulose phthalate, hydroxypropyl-methylcellulose acetate succinate, chitosan and alginates.

Likewise, polysaccharides and polypeptides and other polymers can be fashioned to be temperature sensitive (see, e.g., Okano, “Molecular Design of Stimuli-Responsive Hydrogels for Temporal Controlled Drug Delivery,” in Proceed. Intern. Symp. Control. Rel. Bioact Mater. 22:111-112, Controlled Release Society, Inc., 1995; Hoffman et al., “Characterizing Pore Sizes and Water ‘Structure’ in Stimuli-Responsive Hydrogels,” Center for Bioengineering, Univ. of Washington, Seattle, Wash., p. 828; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305). Representative examples of thermogelling polymers, such as poly(oxyethylene)-poly(oxypropylene) block copolymers (e.g., PLURONIC F127 from BASF Corporation, Mount Olive, N.J.), and cellulose derivatives. Paclitaxel microspheres having lower, traditional loadings have been incorporated into a thermoreversible gel carrier (WO 00/66085).

Exemplary polysaccharides include, without limitation, hyaluronic acid (HA), also known as hyaluronan, and derivatives thereof (see, e.g., U.S. Pat. Nos. 5,399,351, 5,266,563, 5,246,698, 5,143,724, 5,128,326, 5,099,013, 4,913,743, and 4,713,448), including esters, partial esters and salts of hyaluronic acid. For example, an aqueous solution of HA having a non-inflammatory molecular weight (greater than about 900 kDa) and a concentration of about 10 mg/ml would be in the form of a gel. The aqueous solution may further comprise one or more excipients that serve other functions, such as buffering, anti-microbial stabilization, or prevention of oxidation. Microspheres made from, for example, 70% paclitaxel loaded poly(L-lactide), MW=2000, may be incorporated into a 10 mg/ml HA gel as follows. HA, MW=1 MDa, is dissolved in water to a concentration of 20 mg/ml and microparticles are dispersed in water to a concentration in the range of 0.02 to 20 mg/ml. The two phases are combined in equal volumes by mixing (e.g., syringe mixing, using two interconnected luer lok syringes between which the liquids are passed back and forth fifty times), such that the microparticles are evenly distributed throughout the mixture, which has a concentration of 10 mg/ml HA and between 0.1 and 10 mg/ml microparticles, equivalent to 0.07 and 7 mg/ml paclitaxel in a gel carrier.

2. Creams, Ointments and Pastes

Creams, ointments and pastes useful as carriers in certain embodiments compositions of the present invention are may be conventional delivery systems or cosmetic vehicles. Such formulations carriers are described in texts such as Remington's Pharmacetuical Sciences (17th edition, Alfonso Gennaro, 1985, Mack Publishing Co. Easton Pa.).

Creams, ointment and pastes may be formed from or include absorbent ointment bases (e.g., anhydrous lanolin also called Wool Fat USP XVI; Hydrophilic Petrolatum or hydroxystearin sulphate); oleaginous ointment bases (e.g., Ointment USP XI also called “White Ointment” or “Simple Ointment”, Yellow Ointment, Petroleum Jelly also called “Petrolatum”, or White Petroleum Jelly also called “White Petrolatum”); emulsion bases (e.g., Cold Cream, also called Petrolatum Rose Water Ointment USP XVI, Rose Water Ointment, Hydrophilic Ointment) and also includes precursor thereto or ingredients thereof, including but not limited to, for example, acacia, agar, alginic acid, alginic salts, Bentonite, cross-linked polymers of acrylic acid such as CARBOMER (CarboMer, Inc., San Diego, Calif.), carrageenan, cellulose and derivatives thereof, cholesterol, gelatin, sodium lauryl sulphate, TWEEN (available from ICI Americas, Inc., Bridgewater, N.J., under the trade designation TWEEN) and Spans, which are sorbitan esters available from ICI Americas, Inc. including SPAN 20 (sorbitan laurate), SPAN 60 (sorbitan stearate), SPAN 80 (sorbitan oleate), BRIJ surfactants, stearyl alcohol, xanthan gum, mucillages, waxes such as paraffin, beeswax, or spermaceti, polyethylene glycol ointment base, petrolatum, oleic acid, olive oil, and mineral oil.

In certain embodiments, the carrier forms an oil-in-water type emulsion and microparticles dispersed within it. In certain embodiments, the non-aqueous phase of the emulsion comprises at least one of benzyl benzoate, tributyrin, triacetin, mineral oil, olive oil, safflower oil and corn oil. In certain embodiments, the emulsion may be a microemulsion. In other embodiments, the emulsion may be a cream. In yet other embodiments, the emulsion may be a lotion. Microparticles may be incorporated at the time the emulsion is prepared by suspending microparticles into one or both liquid phases prior to emulsification. Alternately, microparticles may be added after the emulsion is formed, by mixing.

Pastes may be formed from any semi-solid vehicle by the inclusion of sufficient solid microparticles. Typically, pastes will have 40% or more solid microparticles in the selected vehicle. Various techniques known to those skilled in the art of compounding may be used to form such a paste, such as mixing by levigation and/or geometric dilution. For example, microparticles may be mixed with White Petrolatum in a 1:1 weight ratio by levigation to produce a suitable paste. The process may be completed by hand or by using an automated or manufacturing process.

3. Tablets and Capsules

In certain embodiments of the invention, a carrier and microspheres may form a composition in the form of a tablet. Tablets may be formed by a number of means and using a number of ingredients known to those skilled in the art, and described in texts such as Remington's Pharmaceutical Sciences (17^{th} edition A. Gennaro ed., Mack Publishing Company 1985, Easton Pa., pp 1605-25). Tablets in these embodiments may be designed to be administered by chewing, swallowing, dissolving under the tongue, injection or insertion into a body cavity. Depending on the application, tablets will therefore be designed having definitive physical properties such as disintegration rate, dissolution rate, friability, hardness and drug dose. To accomplish the required design a number of excipients may be used such as diluents, (e.g., dicalcium phosphate, calcium sulphate, lactose, cellulose, kaolin, mannitol, sodium chloride, sugar, starch, sorbitol, or inositol), binders (e.g., starch, gelatin, sucrose, glucose, dextrose, lactose, natural gums such as sodium alginate, synthetic gums such as Veegum, polyethylene glycol, polyvinylpyrrolidone, or ethyl cellulose), lubricants (e.g., talc, magnesium stearate, or hydrogenated vegetable oil), glidants (e.g., talc or silicone dioxide), disintegrants (e.g., starch, celluloses, aligns, gums, crosslinked polymers, Croscarmelose, or Crospovidone), colorants such as FDand C dyes, flavoring agents, effervescing agents such as sodium bicarbonate, or film or sugar coatings. Tablets may be formulated to provide sustained release, or protection from stomach acid. Microparticles may be added at an appropriate step in the preparation of tablets, such as inclusion into granules, by mixing with powders prior to wet or dry granulation, or by blending microparticles with preexistent granules.

In yet other embodiments, the carrier may be formed as a capsule the interior of which contains microparticles and optionally other excipients and the exterior of which is formed by a shell formed, for example, from gelatin. Capsules may be hard or soft, with the flexibility being modulated by the addition of plasticizers into the shell. Suitable plasticizers include glycerin or sorbitol. Capsules may be formed using techniques, ingredients and methods known to those skilled in the art and described in texts such as Remington's Pharmaceutical Sciences (17^{th} edition A. Gennaro ed., Mack Publishing Company 1985, Easton Pa., pp 1625-30).

4. Suppositories and Inserts

In certain embodiments of the invention, microparticles are contained within a carrier that is a suppository or insert intended to deliver the microparticles into the rectal or vaginal cavities. Such suppositories may be fabricated by conventional means known to those skilled in the art of pharmaceutical compounding. Typically, suppositories will include a solid matrix in which the microparticles are contained. The solid may be comprised of a low melting material such as cocoa butter, mixtures of polyethylene glycol 1000, 4000 and 6000, or glycerinated gelatin so that upon insertion into a body cavity having a temperature of, for example, greater than 32° C., the matrix will melt, releasing the microparticles. Suppositories or inserts comprising microparticles may be fabricated by conventional means by melting the matrix material to form a liquid, mixing in microparticles and compression molding or melt molding the material to form the final composition.

5. Sprays

In certain embodiments of the invention, microparticles are contained within a carrier that is administered as a spray, resulting in aerosol formation, nebulization, suspension of microparticles in a gas (including air), etc. In such embodiments, a spray is meant to include the dispersed system being sprayed, as well as precursors thereto. Sprays may be administered using various devices such as inhalers, nebulizers, syringes equipped with a sprayer, and pressurized canisters equipped with atomizers. Sprays may be inhaled, or applied to a surface such as skin, a serosal or mucosal surface, a wound site, a surgical site, the airways or the throat.

6. Powders

Within the scope of the invention, high loading microparticles may be formed into a powder that may have additional excipients. Powders may be used as a drug delivery system in certain conventional systems such as oral powders for suspension, douche powders, insufflations or dusting powders. Alternately, powders may be used in compounding by a pharmacist in the preparation of pastes, creams or triturations. In the invention, powder compositions comprise microparticles and may further comprise ingredients that impart specific tonicity, pH, dissolution or suspension characteristics. Powders may be packaged as bulk or divided powders or may be contained in a suitable delivery system. Pulmonary delivery systems suitable for the delivery of powders may be used to deliver high drug loading microparticles to the airways.

7. Films

Within yet other aspects of the invention, microparticles may be combined with a carrier to form a film. Preferably, such films are generally less than 5, 4, 3, 2 or 1 mm thick, more preferably less than 0.75 mm or 0.5 mm thick, and most preferably less than 500 μm. Such films are preferably flexible with a good tensile strength (e.g., greater than 50, preferably greater than 100, and more preferably greater than 150 or 200 N/cm²), good adhesive properties (i.e., readily adheres to moist or wet surfaces), and have controlled permeability.

8. Tissue Sealants

In certain embodiments, high drug loaded microspheres of the present invention may also be combined with tissue sealants. As used herein, the term “sealant” refers to a material that decreases or prevents the migration of fluid from or into a surface such as a tissue surface. Sealants are typically formed by the application of precursor molecules to a tissue followed by local polymerization. Sealants may also be used to adhere materials together, either when applied between the materials and then polymerized, or when used to jointly embed materials. Generally, surgical sealants are absorbable materials used primarily to control internal bleeding and to seal tissue.

Sealant material and devices for delivering sealant materials for use in the instant invention are described, e.g., in U.S. Pat. Nos. 6,624,245; 6,534,591; 6,495,127; 6,482,179; 6,458,889; 6,323,278; 6,312,725; 6,280,727; 6,277,394; 6,166,130; 6,110,484; 6,096,309; 6,051,648; and 5,874,500; 6,063,061; 5,895,412; 5,900,245; and 6,379,373.

Sealants that may be combined with one or more drugs contained at least partly in highly loaded microparticles include tissue adhesives (e.g., cyanoacryates and cross-linked poly(ethylene glycol)-methylated collagen compositions) and sealants, including commercially available products, such as COSEAL (Cohesion Technologies, Inc., Palo Alto, Calif.), FLOSEAL (Fusion Medical Technologies, Inc., Fremont, Calif.); SPRAYGEL or a variation thereof (Confluent Surgical, Inc., Boston Mass.); and absorbable sealants for use in lung surgery, such as FOCALSEAL (Genzyme BioSurgery, Cambridge, Mass.).

9. Scaffolds

The compositions of the present invention may be fashioned in a wide variety of forms and may include a scaffold in addition to drug loaded microparticles, and optionally in addition to another carrier.

In compositions including a scaffold, microparticles may be applied onto the exterior and/or interior surfaces of the scaffold resulting in a solid or semi-solid structure often having a defined geometry.

Suitable scaffolds include metallic medical implants such as stents, screws, pins, plates or artificial joints; fabrics such as gauze; porous matrices such as sponges made of gelatin (e.g., GELFOAM from Amersham Health), or cellulose or derivatives thereof (e.g., SEPRAFILM); biologically derived matrices such as semi-synthetic heart valves from a mammalian source (e.g., porcine source), autologous or synthetic tissue grafts such as skin or bone; orthopedic implants such as those made of biodegradable polymers such as poly(L-lactide); sutures; catheters (e.g., balloon catheters); implants made, e.g., of collagen, polyethylene, silicone, ethylene vinyl acetate copolymer, fluorinated polyethylene derivatives (e.g., TEFLON), or a polyurethane; grafts; stent-grafts; hydrogels; tissue sealants, shunts; aneurysm coils; bandages; or implantable brachytherapy devices.

The scaffold may facilitate delivery of the drug to its intended site of action, and at the same time, the scaffold also may provide other therapeutic effects. For example, a stent may be used to deliver a drug to a blood vessel and to open the blood vessel having a reduced lumen size due to atherosclerosis, a suture may be used to deliver a drug to a wound site while at the same time providing for mechanical closure of the wound site, or a skin graft could be used to deliver a drug to a burn while at the same time promoting tissue regeneration. Because of the possibility of a dual therapeutic action of a composition that includes drug loaded microparticles and a scaffold, certain embodiments of the invention include a drug and a scaffold wherein the drug is intended to have a therapeutic effect which is complementary, additive or synergistic to the therapeutic effect expected to be achieved by the scaffold itself, yielding an improvement over conventional therapy.

a. Catheters and Balloon Catheters

In certain embodiments of the invention, microparticles or compositions comprising microparticles may be combined with a scaffold that is a catheter designed to deliver a fluid or a surgical device into a lumen within the body. Suitable catheters may be intended for use in the cardiovascular system or the genitourinary tract. In certain other embodiments, the catheter may be equipped with a balloon designed to temporarily occlude a lumen and optionally permanently alter the luminal area, such as an angioplasty balloon. Catheters suitable for use as a scaffold may be fabricated of polymers such as silicone, ethylene vinyl acetate, polyurethanes and may comprise other polymers such as polyethylene, or polytetrafluoroethylene or lubricious coating polymers. Numerous suitable catheters are commercially available from a wide variety of vendors including Boston Scientific Corporation (Natick, Mass.), Cordis Corporation (Miami Lakes, Fla.), C.R. Bard Inc. (Murray Hill, N.J.), and Baxter Healthcare Corporation (Deerfield, Ill.).

Stents may be used as a scaffold by positioning high drug loading microparticles, optionally using a carrier such as a gel or hydrogel, onto the surface of the catheter, or into pores within catheter wall. The microparticles, and optionally a carrier, may be applied by means such as dipping, spraying or painting. Optionally, microparticles may be incorporated at the time of catheter manufacture. In the case of balloon catheters, microparticles could be incorporated into the device such that the balloon is inflated with a carrier containing microparticles. The balloon catheter may be so constructed as to allow the microparticles to pass through the inflated balloon, being delivered to the lumen wall.

b. Stents

In certain embodiments of the invention, microspheres or a composition comprising microspheres may be combined with a scaffold that is a stent designed to maintain the opening of a lumen within the body.

A wide variety of stents may be developed to contain and/or release the high loading microparticles provided herein, including esophageal stents, gastrointestinal stents, vascular stents, biliary stents, colonic stents, pancreatic stents, ureteric and urethral stents, lacrimal stents, Eustachian tube stents, fallopian tube stents, nasal stents, sinus stents and tracheal/bronchial stents. Stents that can be used in the present invention include metallic stents, which may be fabricated of materials comprising metals, such as, titanium, nickel, or suitable alloys such as steel or nickel-tatnium, polymeric stents, biodegradable stents and covered stents. Stents may be self-expandable or balloon-expandable, composed of a variety of metal compounds and/or polymeric materials, fabricated in innumerable designs, used in coronary or peripheral vessels, composed of degradable and/or nondegradable components, fully or partially covered with vascular graft materials or “sleeves,” and can be bare metal or drug-eluting.

Stents may be readily obtained from commercial sources, or constructed in accordance with well-known techniques. Representative examples of stents include those described in U.S. Pat. No. 4,768,523, entitled “Hydrogel Adhesive”; U.S. Pat. No. 4,776,337, entitled “Expandable Intraluminal Graft, and Method and Apparatus for Implanting and Expandable Intraluminal Graft”; U.S. Pat. No. 5,041,126 entitled “Endovascular Stent and Delivery System”; U.S. Pat. No. 5,052,998 entitled “Indwelling Stent and Method of Use”; U.S. Pat. No. 5,064,435 entitled “Self-Expanding Prosthesis Having Stable Axial Length”; U.S. Pat. No. 5,089,606, entitled “Water-insoluble Polysaccharide Hydrogel Foam for Medical Applications”; U.S. Pat. No. 5,147,370, entitled “Nitinol Stent for Hollow Body Conduits”; U.S. Pat. No. 5,176,626, entitled “Indwelling Stent”; U.S. Pat. No. 5,213,580, entitled “Biodegradable Polymeric Endoluminal Sealing Process”; and U.S. Pat. No. 5,328,471, entitled “Method and Apparatus for Treatment of Focal Disease in Hollow Tubular Organs and Other Tissue Lumens.” Drug delivery stents are described, e.g., in PCT Publication No. WO 01/01957 and U.S. Pat. Nos. 6,165,210; 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,766,237; 5,769,883; 5,735,811; 5,700,286; 5,683,448; 5,679,400; 5,665,115; 5,649,977; 5,637,113; 5,591,227; 5,551,954; 5,545,208; 5,500,013; 5,464,450; 5,419,760; 5,411,550; 5,342,348; 5,286,254; and 5,163,952. Removable drug-eluting stents are described, e.g., in Lambert, T. (1993) J. Am. Coll. Cardiol. 21: 483A. Moreover, the stent may be adapted to release the desired agent at only the distal ends, or along the entire body of the stent. Self-expanding stents that can be used include the coronary WALLSTENT and the SciMED RADIUS stent from Boston Scientific, Natick, Mass. Examples of balloon expandable stents that can be used include the CROSSFLEX stent, BX-VELOCITY stent and the PALMAZ-SCHATZ Crown and Spiral stents from Cordis, the V-FLEX PLUS stent by Cook, Inc., the NIR and EXPRESS stents by Boston Scientific Corp., the ACS MULTILINK and MULTILINK PENTA stents by Guidant Corp., the Coronary Stent S670 and S7 by Medtronic AVE, and the PAS stent by Progressive Angioplasty Systems Inc. In addition to using the more traditional stents, stents that are specifically designed for drug delivery can be used. Examples of these specialized drug delivery stents as well as traditional stents include those from Conor Medsystems (Palo Alto, Calif.) (U.S. Pat. Nos. 6,527,799; 6,293,967; 6,290,673; 6,241,762; U.S. Patent Application Nos. 2003/0199970 and 2003/0167085; and PCT Publication No. WO 03/015664). Other types of stents for use as scaffolds include coronary stents such as, for example, AVE Micro stent, FREEDOM stent, or the SciMED self expanding stent. Additional exemplary coronary stents are listed in the Handbook of Coronary Stents (PW Serruys, Mosby, St Louis, 1997). Suitable stents may also be designed or used in peripheral blood vessels, the bile duct (e.g., DYNALINK or OMNILINK from Advanced Cardiovascular Systems, Inc., Santa Clara, Calif.), the duodenum (e.g., WALLSTENT), the esophagus (e.g., WALLSTENT), or the trachea or bronchia (e.g., ULTRAFLEX stent from Boston Scientific Co.).

Stent scaffolds may also include polymers such as polyurethanes or polyethylene (van Berkel et al, Endoscopy 2003(35) 478-82), poly(L-lactide) (Su et al., Ann. Biomed Eng 2003(31) 667-77; Tsuji et al., Int. J. Cardiovasc. Intervent 2003(5) 13-6), bioresorbable polymers (Eberhart et al., J Biomater. Sci. Polym. Ed 2003(14) 299-312) or polytetrafluoroethylene (Gyenes et al., Can J Cardiol. 2003(19) 569-71).

Stents may be used as a scaffold by depositing microparticles having a high loading of drug, optionally using a carrier such as a gel or hydrogel, onto the surface of the stent, into a depression within the stent structure, into gaps between the stent tines, or into holes formed by means such as drilling into the stent surface (as described in, e.g., US 2003/0068355A1). The microparticles and optional carriers may be applied to the stent by means such as dipping, spraying or painting.

c. Grafts and Stent-Grafts

A wide variety of stent grafts may be utilized as a scaffold within the context of the present invention, depending on the site and nature of treatment desired. Stent grafts may be, for example, bifurcated or tube grafts, cylindrical or tapered, self-expandable or balloon-expandable, unibody, or, modular. Moreover, the stent graft may be adapted to release the desired agent at only the distal ends, or along the entire body of the stent graft. The graft portion of the stent may be composed of a textile, polymer, or other suitable material such as biological tissue. Representative examples of suitable graft materials include textiles such as nylon, acylonitrile polymers, such as ORLON from E. I. Du Pont De Nemours and Company, Wilmington, Del., polyester, such as DACRON from E. I. Du Pont De Nemours and Company, Wilmington, Del.), or woven polytetrafluoroethylene (e.g., TEFLON from E. I. Du Pont De Nemours and Company, Wilmington, Del.), and non-textiles such as expanded polytetrafluoroethylene (PTFE). Representative examples of stent grafts, and methods for making and utilizing such grafts are described in more detail in U.S. Pat. Nos. 5,810,870; 5,776,180; 5,755,774; 5,735,892; 5,700,285; 5,723,004; 5,718,973; 5,716,365; 5,713,917; 5,693,087; 5,683,452; 5,683,448; 5,653,747; 5,643,208; 5,639,278; 5,632,772; 5,628,788; 5,591,229; 5,591,195; 5,578,072; 5,578,071; 5,571,173; 5,571,171; 5,522,880; 5,405,377; and 5,360,443.

A stent grafts used as a scaffold in the present invention may be coated with, or otherwise adapted to release an agent that induces adhesion to vessel walls. Such an agent, such as a profibrotic agent, may be contained with a high loading in microparticles and the microparticles attached to the graft surface for example by electrostatic charge and optionally a “glue” or reinforcing layer such as a hydrogel may be added. Alternatively, microparticles may be incorporated into a carrier such a s a gel or polymer solution which is coated onto the scaffold by either spraying the stent graft with a polymer/drug film, or by dipping the stent graft into the carrier solution. In another embodiment, microparticles may be incorporated into the spaces in the weave of the fabric on the stent graft, or may be incorporated into the fibers themselves, to facilitate weaving the microparticles into the material.

Similarly, a wide range of grafts may also be employed as a scaffold. Synthetic grafts are commonly made of expanded TEFLON but other suitable textiles may be used, as listed above for stent grafts. Microparticles may be incorporated into grafts in a manner similar to that disclosed for stent grafts.

d. Gauze and Bandages

In certain embodiments of the invention, microparticles or a composition comprising microparticles may be combined with a scaffold that is a bandage or a fabric, such as a gauze. The gauze or bandage may be so designed as to be useful for covering a wound for example on the skin, or to be used as a packing into a internal wound or to be used as an adjunct in a surgical procedure. Gauze (e.g., a woven or non-woven mesh material) may be formed of materials such as cotton, rayon or polyester fibers. Bandages may include adhesive and non-adhesive bandages. Microparticles may be incorporated onto the exterior surface of such a scaffold, or into the porous structure (e.g., within the weave) of a gauze.

e. Sutures

In certain embodiments of the invention, microparticles or a composition comprising microparticles may be combined with a scaffold that is a suture designed to effect the closure of a wound or incision, or to fix a tissue or medical device or implant in place. Such a suture may be fabricated of materials and by methods known to those skilled in the art. Suitable sutures may comprise for example biodegradable polymers such as poly(glycolide), poly(lactide) or co-polymers thereof. Sutures may be formed comprising materials such as silk or catgut, nylon, or polypropylene. Suitable sutures may be braided or monofilamentous. Microparticles may be affixed onto sutures by incorporation into a carrier that adheres to the surface of the suture. Microparticles may be introduced within the suture at the time of its manufacture. Microparticles may alternatively be applied to the suture immediately prior to its use, for example by dipping the suture into a medium containing the microparticles, allowing them to adhere to the surface.

f. Sponges, Pledgets and Implantable Porous Membranes

In certain embodiments of the invention, microparticles or a composition comprising microparticles may be combined with a scaffold that is a sponge, pledget or implantable porous membrane designed to allow for the ingress of body fluids or tissues after implantation. Such a device may be fabricated of materials and by methods known to those skilled in the art. Such porous materials may be made of materials such as collagen, gelatin (e.g., GELFOAM), HA and derivatives thereof (e.g., SEPRAMESH or SEPRAFILM from Genzyme Corporation, Cambridge, Mass.), and cellulose.

In certain embodiments, the sponge may be a pledget comprising materials such as cotton, cellulose, gelatin, or TEFLON. Microparticles may be incorporated into a pledget by suspending them in a carrier and soaking the pledget in the suspension, taking up the liquid and the suspended microparticles. Microparticles may be loaded in this manner immediately prior to use of the composition, or at an earlier time of manufacture. In certain embodiments, the liquid carrier may then be removed by methods such as drying are using pressure to expel the liquid. In certain embodiments, the carrier may be a semi-solid such as a gel or ointment. The pledget may be implanted or used topically or on a wound surface.

In certain embodiments, the scaffold may be a wound dressing, including those in the form of a membrane, a fabric material (exemplified by 3M Medipore products), a bandage or a hydrogel structure, and a foam structure (e.g., those comprising polyurethane). Suitable wound dressings include cellulosic materials (e.g. those exemplified by Aquacel Hydrofiber, which comprises sodium carboxymethylcellulose), nylon fabrics, silicone hydrogels, and oil emulsion dressings (exemplified by Adaptic and Invacare® Oil Emulsion Dressing).

g. Orthopedic Implants

In certain embodiments of the invention, microparticles or a composition that comprises microparticles may be combined with a scaffold that is an orthopedic implant designed to provide stability or articulation to the skeletal system, including joints. Implants include pins, screws, plates, grafts (including allografts) of, for example, tendons, anchors, total joint replacement devices, such as artificial knees and hips. The orthopedic implant may be fabricated of materials that include metals, such as, for example, titanium, nickel, or suitable alloys such as steel or nickel-titanium. Suitable orthopedic implants may also comprise polymers such as polyurethanes or polyethylene, polycarbonate, polyacrylates (e.g., polymethyl methacrylate), poly(L-lactide) or polytetrafluoroethylene. Orthopedic implants may also include bone implants that include tricalcium phosphate or hydroxyapatite.

Exemplary orthopedic devices which are suitable scaffolds in certain embodiments of this invention are described in The Radiology of Orthopaedic Implants An Atlas of Techniques and Assessment by Andrew A. Freiberg (Editor), William, M.D. Martel, Mosby Publishing (2001) ISBN 0323002226. The microparticles and optional carriers may be applied to the orthopedic devices by means such as dipping, spraying or painting.

h. Tissue Fillers

In certain embodiments of the invention, microparticles or a composition that comprises microparticles may be combined with a scaffold that is a tissue filler such as dermal fillers and soft tissue implants or with material(s) for forming a tissue filler to form a drug loaded tissue filler.

Tissue fillers such as soft tissue implants are used in a variety of cosmetic, plastic, and reconstructive surgical procedures and may be delivered to many different parts of the body, including, without limitation, the face, nose, jaw, breast, chin, buttocks, chest, lip, and cheek. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Soft tissue implants may be used for the augmentation of tissue for cosmetic (aesthetic) enhancement or in association with reconstructive surgery following disease or surgical resection. Representative examples of soft tissue implants that can be coated with, or otherwise constructed to contain and/or release microparticles or therapeutic agents present in the microparticles (e.g., anti-fibrotic agents and local anesthetics) include, e.g., saline breast implants, silicone breast implants, triglyceride-filled breast implants, chin and mandibular implants, nasal implants, cheek implants, lip implants, and other facial implants, pectoral and chest implants, malar and submalar implants, and buttocks implants.

Soft tissue implants have numerous constructions and may be formed of a variety of materials, such as to conform to the surrounding anatomical structures and characteristics. In one aspect, soft tissue implants suitable for combining with a fibrosis-inhibitor are formed from a polymer such as silicone, poly(tetrafluoroethylene), polyethylene, polyurethane, polymethylmethacrylate, polyester, polyamide and polypropylene. Soft tissue implants may be in the form shell (or envelope) that is filled with a fluid material such as saline.

In one aspect, soft tissue implants include or are formed from silicone or dimethylsiloxane. Silicone implants can be solid, yet flexible and very durable and stable. They are manufactured in different durometers (degrees of hardness) to be soft or quite hard, which is determined by the extent of polymerization. Short polymer chains result in liquid silicone with less viscosity, while lengthening the chains produces gel-type substances, and cross-linking of the polymer chains results in high-viscosity silicone rubber. Silicone may also be mixed as a particulate with water and a hydrogel carrier to allow for fibrous tissue ingrowth. These implants are designed to enhance soft tissue areas rather than the underlying bone structure. In certain aspects, silicone-based implants (e.g., chin implants) may be affixed to the underlying bone by way of one or several titanium screws. Silicone implants can be used to augment tissue in a variety of locations in the body, including, for example, breast, nasal, chin, malar (e.g., cheek), and chest/pectoral area. Silicone gel with low viscosity has been primarily used for filling breast implants, while high viscosity silicone is used for tissue expanders and outer shells of both saline-filled and silicone-filled breast implants. For example, breast implants are manufactured by both Inamed Corporation (Santa Barbara, Calif.) and Mentor Corporation (Santa Barbara, Calif.).

In another aspect, soft tissue implants include or are formed from poly(tetrafluoroethylene) (PTFE). In certain aspects, the poly(tetrafluoroethylene) is expanded polytetrafluoroethylene (ePTFE). PTFE used for soft tissue implants may be formed of an expanded polymer of solid PTFE nodes with interconnecting, thin PTFE fibrils that form a grid pattern, resulting in a pliable, durable, biocompatible material. Soft tissue implants made of PTFE are often available in sheets that may be easily contoured and stacked to a desired thickness, as well as solid blocks. These implants are porous and can become integrated into the surrounding tissue that aids in maintaining the implant in its appropriate anatomical location. PTFE implants generally are not as firm as silicone implants. Further, there is less bone resorption underneath ePTFE implants as opposed to silicone implants. Soft tissue implants composed of PTFE may be used to augment tissue in a variety of locations in the body, including, for example, facial, chest, lip, nasal, and chin, as well as the mandibular and malar region and for the treatment of nasolabial and glabellar creases. For example, GORE-TEX (W.L. Gore & Associates, Inc., Newark, Del.) is an expanded synthetic PTFE that may be used to form facial implants for augmentation purposes.

In yet another aspect, soft tissue implants include or are formed from polyethylene. Polyethylene implants are frequently used, for example in chin augmentation. Polyethylene implants can be porous, such that they may become integrated into the surrounding tissue, which provides an alternative to using titanium screws for stability. Polyethylene implants may be available with varying biochemical properties, including chemical resistance, tensile strength, and hardness. Polyethylene implants may be used for facial reconstruction, including malar, chin, nasal, and cranial implants. For example, Porex Surgical Products Group (Newnan, Ga.) makes MEDPOR, which is a high-density, porous polyethylene implant that is used in facial reconstruction. The porosity allows for vascular and soft tissue ingrowth for incorporation of the implant.

In yet another aspect, soft tissue implants include or are formed from polypropylene. Polypropylene implants are a loosely woven, high density polymer having similar properties to polyethylene. These implants have good tensile strength and are available as a woven mesh, such as PROLENE (Ethicon, Inc., Sommerville, N.J.) or MARLEX (C.R. Bard, Inc., Billerica, Mass.). Polypropylene implants may be used, for example, as chest implants.

In yet another aspect, soft tissue implants include or are formed from polyamide. Polyamide is a nylon compound that is woven into a mesh that may be implanted for use in facial reconstruction and augmentation. These implants are easily shaped and sutured and undergo resorption over time. SUPRAMID and SUPRAMESH(S. Jackson, Inc., Minneapolis, Minn.) are nylon-based products that may be used for augmentation; however, because of their resorptive properties, their application is limited.

In yet another aspect, soft tissue implants include or are formed from polyester. Nonbiodegradable polyesters, such as MERSILENE Mesh (Ethicon, Inc.) and DACRON (available from Invista, Wichita, Kans.), may be suitable as implants for applications that require both tensile strength and stability, such as chest, chin and nasal augmentation.

In yet another aspect, soft tissue implants include or are formed from polymethylmethacrylate. These implants have a high molecular weight and have compressive strength and rigidity even though they have extensive porosity. Polymethylmethacrylate, such as Hard Tissue Replacement (HTR) polymer made by U.S. Surgical Corporation (Norwalk, Conn.), may be used for chin and malar augmentation as well as craniomaxillofacial reconstruction.

In yet another aspect, soft tissue implants include or are formed from polyurethane. Polyurethane may be used as a foam to cover breast implants. This polymer promotes tissue ingrowth resulting in low capsular contracture rate in breast implants.

In yet another aspect, soft tissue implants include or are formed from collagen. Such implants may be used as dermal fillers.

Examples of commercially available polymeric soft tissue implants suitable for use in combination with a fibrosis-inhibitor include silicone implants from Surgiform Technology, Ltd. (Columbia Station, Ohio); ImplantTech Associates (Ventura, Calif.); Inamed Corporation (Santa Barbara, Calif.; see M766A Spectrum Catalog); Mentor Corporation (Santa Barbara, Calif.); and Allied Biomedical (Ventura, Calif.). Saline filled breast implants are made by both Inamed and Mentor and may also benefit from implantation in combination with a fibrosis inhibitor. Commercially available poly(tetrafluoroethylene) soft tissue implants suitable for use in combination with a fibrosis-inhibitor include poly(tetrafluoroethylene) cheek, chin, and nasal implants from W. L. Gore & Associates, Inc. (Newark, Del.). Commercially available polyethylene soft tissue implants suitable for use in combination with a fibrosis-inhibitor include polyethylene implants from Porex Surgical Inc. (Fairburn, Ga.) sold under the trade name MEDPOR Biomaterial. MEDPOR Biomaterial is composed of porous, high-density polyethylene material with an omni-directional latticework of interconnecting pores, which allows for integration into host tissues.

In certain embodiments, microspheres or compositions comprising microspheres may be applied to the exterior surface of a tissue filler. In certain embodiments, microspheres or compositions comprising microspheres may be combined with ingredients of a tissue filler to form a drug loaded tissue filler in which microspheres are distributed within the resulting tissue filler. Exemplary methods are provided in the examples below.

### Methods for Making Compositions and Kits

In one aspect, the present invention provides methods for making polymers useful in preparing high drug loaded microparticles, microparticles, compositions and medical devices that comprises microparticles.

In certain embodiments the present invention provides methods for producing a polyester that may be used in making high drug loaded microparticles. Such methods comprise polymerizing a composition comprising one or more of the monomers selected from the group consisting of lactide, lactic acid, glycolide, glycolic acid, ε-caprolactone, γ-caprolactone, hydroxyvaleric acid, hydroxybutyric acid, β-butyrolactone, gamma-butyrolactone, gamma-valerolactone, γ-decanolactone, δ-decanolactone, trimethylene carbonate, 1,4-dioxane-2-one and 1,5-dioxepan-2-one using a polymerization initiator, wherein the polymerization initiator is salicylic acid. Detailed description of these methods may be found in Example 1.

In certain embodiments, the present invention provides a method for making high drug loading microparticles. Various known methods of microsphere manufacture may be adapted to incorporate high percentage of drug loading include a) phase separation followed by solvent evaporation in dispersions such as o/o, w/o, o/w or w/o/w (o=oil, w=water), b) use of super critical fluids c) coacervation, d) melt dispersions, e) spray drying, f) spray congealing, or g) suspension coating. Exemplary methods include those in U.S. Pat. Nos. 4,652,441; 5,100,669; 4,438,253 and 5,665,428. The amount of drugs appropriate for making a particular high drug loaded microparticle may be calculated according to Example 3.

In certain embodiments, the present invention provides methods for preparing microspheres by combining a drug and a polymer in a suitable processing solvent (e.g., dichloromethane, chloroform, ethyl acetate, acetone, and methanol). The resultant mixture may be dispersed to form droplets by, for example, (a) dispersing, with the aid of stirring, the mixture into a liquid containing a stabilizer, wherein the liquid is substantially immiscible with the processing solvent; or (b) spraying the mixture through a nozzle into a heated circulating gas such that droplets are formed. Subsequently, the processing solvents are removed from the formed droplets by evaporation or other means of phase separation of the solvent, leaving solid microparticles. Detailed description of exemplary methods may be found in Examples 3 and 5.

In certain other embodiments, microparticles may be formed according to the process described below. The drug may be combined with a polymer and, optionally, a suitable processing solvent or solvents (as described above) and formed into a liquid or semi-solid composition by, for example, dissolving or melting the components. The liquid composition then is poured, extruded, or injected into or onto a suitable substrate such as a mold, liner or rollers, and the solvent(s) removed by heating and/or reducing pressure, resulting in at most only residual levels of the solvent in the mixture. The particle size of the solid composition may be reduced using any suitable method, such as grinding and milling.

In addition to the methods and compositions described above, additional components/excipients may be included to produce compositions of the present invention. For instance, in certain embodiments, the compositions of this invention may further include water and/or have a pH of about 3-9. In certain embodiments, compositions comprise a drug such as an anti-microtubule agent, an agent that enhances the dispersability of the drug in an aqueous medium, and at least one of polypeptide or a polysaccharide. In addition to any of the compositions described herein, any pharmaceutically or veterinarilly acceptable vehicle, diluent, or excipient, may be included, optionally with other components. Pharmaceutically or veterinarilly acceptable excipients for therapeutic use are well known in the pharmaceutical art, and are described, for example, in Remington: The Science and Practice of Pharmacy (formerly Remington's Pharmaceutical Sciences), Lippincott Williams and Wilkins (A. R. Gennaro, ed., 20^{th} Edition, 2000) and in CRC Handbook of Food, Drug, and Cosmetic Excipients, CRC Press (S. C. Smolinski, ed., 1992). For example, sterile saline, 5% dextrose solution, and phosphate buffered saline at physiological pH may be used.

Preservatives, bacteriostatic agents, bactericidal agents, antioxidants, stabilizers, dyes and/or flavoring agents may also be provided in the composition. For example, in certain embodiments, compositions of the present invention may include one or more preservatives or bacteriostatic agents present in an effective amount to preserve the compositions and/or inhibit bacterial growth in the compositions. Exemplary agents include without limitation bismuth tribromophenate, methyl hydroxybenzoate, bacitracin, ethyl hydroxybenzoate, propyl hydroxybenzoate, erythromycin, chlorocresol, benzalkonium chlorides, paraoxybenzoic acid esters, chlorobutanol, benzylalcohol, phenethyl alcohol, dehydroacetic acid, sorbic acid, etc. In certain embodiments, the compositions of the present invention include one or more bactericidal (also known as bacteriacidal) agents.

In certain embodiments, the compositions of the present invention include one or more antioxidants, present in an effective amount. Examples of the antioxidant include sulfites and ascorbic acid.

In certain embodiments, the compositions of the present invention include one or more coloring agents, also referred to as dyestuffs, which will be present in an effective amount to impart observable coloration to the composition. Examples of coloring agents include dyes suitable for food such as those known as F. D. and C. dyes, and natural coloring agents such as grape skin extract, beet red powder, beta carotene, annato, carmine, turmeric, paprika, and so forth.

In certain embodiments, the compositions of the present invention may include an excipient that is a wax. A variety of natural and synthetic waxes may be used to prepare microparticles in accordance with the invention. Natural waxes may, for example, be derived from animals (e.g. beeswax), vegetables (e.g., carnauba), or minerals (e.g., fossil and petroleum waxes, such as paraffin and microcrystalline wax). Synthetic waxes include hydrocarbon waxes and waxes prepared from ethylenic polymers and polyol ether-esters (e.g. sorbitol). Other types of waxes that may be used to prepare high drug loaded microparticles include esters of fafty acids and alcohols, such as semi-synthetic glyceryl esters.

In certain embodiments, still other excipients may be added to impart specific properties to microparticles or compositions that comprise the microparticles. Such excipients may include binders to form granules, radioactive, radioopaque or X-ray opaque materials such as tantalum, or MRI contrast agents for ease of visualization in a clinical setting, pore formers, density adjusting materials, osmotic pressure adjusting materials, or degradation rate modifiers such as acids or bases.

The microparticles produced according to the present invention are generally between about 0.5 μm to about 1000 μm in size, such as between about between about 0.5 μm and about 500 μm, between about 0.5 μm to about 200 μm, between about 0.5 μm and about 100 μm, between about 0.5 μm and about 50 μm, between about 0.5 μm and about 25 μm, between about 0.5 μm and about 10 μm, or between about 1 μm and about 10 μm. The optimal sizes of the microparticles may be determined by the desired drug release properties and the particular applications. In certain embodiments, the microparticles or microspheres have an average diameter of at least about 0.5 μm, 1 μm, 5 μm, 10 μm, 20 μm, 50 μm or 100 μm. In certain embodiments, the microparticles have a preferred average diameter of no more than about 5 μm, 10 μm, 20 μm, 50 μm, 100 μm, 150 atm, 250 μm, 500 μm, or 1000 μm. The microparticles and microspheres of the invention may have an average diameter of between about 0.5 μm and about 1000 μm, between about 0.5 μm and about 500 μm, between about 0.5 μm to about 200 μm, between about 0.5 μm and about 100 μm, between about 0.5 μm and about 50 μm, between about 0.5 μm and about 25 μm, between about 0.5 μm and about 10 μm, or between about 1 μm and about 10 μm.

Exemplary microparticles include, but are not limited to, microparticles that comprise greater than 65%, 70%, 75%, 80%, 85%, 90% or 95% (w/w) of paclitaxel or its analogue or derivative and a polyester, microparticles that comprise greater than 65%, 70%, 75%, 80%, 85%, 90% or 95% (w/w) of paclitaxel or its analogue or derivative and polylactide, microparticles that comprise greater than 65%, 70%, 75%, 80%, 85%, 90% or 95% (w/w) of paclitaxel or its analogue or derivative and a lactide copolymer, and microparticles that comprise greater than 65%, 70%, 75%, 80%, 85%, 90% or 95% (w/w) of paclitaxel or its analogue or derivative and polylactide-co-glycolide.

Additional exemplary microparticles include, but are not limited to, microparticles that comprise greater than 50%, 55%, 60%, 65%, 70%, 75%, 80%, 85%, 90% or 95% (w/w) of lidocaine or its analogue or derivative and a polyester, microparticles that comprise greater than 50%, 55%, 60%, 65%, 70%, 75%, 80%, 85%, 90% or 95% (w/w) of lidocaine or its analogue or derivative and polylactide, microparticles that comprise greater than 50%, 55%, 60%, 65%, 70%, 75%, 80%, 85%, 90% or 95% (w/w) of lidocaine or its analogue or derivative and a lactide copolymer, and microparticles that comprise greater than 50%, 55%, 60%, 65%, 70%, 75%, 80%, 85%, 90% or 95% (w/w) of lidocaine or its analogue or derivative and polylactide-co-glycolide.

Additional exemplary microparticles include, but are not limited to, microparticles that comprise greater than 50%, 55%, 60%, 65%, 70%, 75%, 80%, 85%, 90% or 95% (w/w) of naproxen and a polymer selected from the group consisting of polyesters, polyethers, polylactide, lactide copolymers, polylactide-co-glycolide).

Additional exemplary microparticles include, but are not limited to, microparticles that comprise greater than 50%, 55%, 60%, 65%, 70%, 75%, 80%, 85%, 90% or 95% (w/w) of hydrocortisone 21-caprylate and a polymer selected from the group consisting of polyesters, polyethers, polylactide, lactide copolymers, polylactide-co-glycolide).

Additional exemplary microparticles include, but are not limited to, microparticles that comprise greater than 50%, 55%, 60%, 65%, 70%, 75%, 80%, 85%, 90% or 95% (w/w) of erythromycin and a polymer selected from the group consisting of polyesters, polyethers, polylactide, lactide copolymers, polylactide-co-glycolide).

Additional exemplary microparticles include, but are not limited to, microparticles that comprise greater than 50%, 55%, 60%, 65%, 70%, 75%, 80%, 85%, 90% or 95% (w/w) of mycophenolic acid and a polymer selected from the group consisting of polyesters, polyethers, polylactide, lactide copolymers, polylactide-co-glycolide).

Additional exemplary microparticles include, but are not limited to, microparticles that comprise greater than 50%, 55%, 60%, 65%, 70%, 75%, 80%, 85%, 90% or 95% (w/w) of phenyloin and a polymer selected from the group consisting of polyesters, polyethers, polylactide, lactide copolymers, polylactide-co-glycolide).

An exemplary microparticle may comprise a polymer having a number average molecular weight (Mₙ) of less than 67,000 g/mol and a drug, wherein the drug is present in the microparticle at a concentration of greater than 60% (weight of drug/weight of microparticle).

Another exemplary microparticle may comprise a polymer having a weight average molecular weight (M_{w}) of less than 100,000 g/mol and a drug, wherein the drug is present in the microparticle at a concentration of greater than 60% (weight of drug/weight of microparticle). In certain embodiments, the polymer may have a M_{w} of less than about 50,000 g/mol or of less than about 10,000 g/mol.

Another exemplary microparticle may comprise a polymer and a drug has a water solubility of less than 10% (weight of drug/volume of water) at 25° C., and wherein the drug is present in the microparticle at a concentration of greater than 60% (weight of drug/weight of microparticle). For example, the drug may have a water solubility of less than 2.5% (weight drug/volume of water) at 25° C., less than 1% (weight drug/volume of water) at 25° C., less than 0.5% (weight drug/volume of water) at 25° C., or less than 0.1% (weight drug/volume of water) at 25° C.

Another exemplary microparticle may comprise a polymer and a drug, wherein the drug has at least 23 carbon atoms, and wherein the drug is present in the microparticle at a concentration of greater than 60% (weight of drug/weight of microparticle).

Another exemplary microparticle may comprise a polymer and a drug, wherein the drug has a molecular weight of greater than 445 g/mol, and wherein the drug is present in the microparticle at a concentration of greater than 60% (weight of drug/weight of microparticle).

Another exemplary microparticle may comprise a polymer and a drug, wherein the drug has a molecular weight of less than 180 g/mol, and wherein the drug is present in the microparticle at a concentration of greater than 50% (weight of drug/weight of microparticle). In certain embodiments, the drug may be present in the microparticle at a concentration of greater than 60% (weight of drug/weight of microparticle).

Another exemplary microparticle may comprise a polyester having a Mₙ of less than 67,000 g/mol and a drug, wherein the drug is present in the microparticle at a concentration of greater than 50% (weight of drug/weight of microparticle).

Another exemplary microparticle may comprise a polyamide and a drug, wherein the drug is present in the microparticle at a concentration of greater than 60% (weight of drug/weight of microparticle).

Another exemplary microparticle may comprise a wax and a drug, wherein the drug is present in the microparticle at a concentration of greater than 60% (weight of drug/weight of microparticle).

Another exemplary microparticle may comprise a polysaccharide and a drug, wherein the drug is present in the microparticle at a concentration of greater than 70% (weight of drug/weight of microparticle).

In certain embodiments, the present application provides a method for making a microparticle composition. Such a method may comprise combining microparticles with a carrier (including a scaffold). In certain related embodiments, the present application provides a method for making a drug loaded medical device. Such a method may comprise combining a medical device (which may function as a scaffold) and high drug loaded microparticles.

Within certain embodiments, microparticles or compositions comprising microparticles are biocompatible. Further, in certain embodiments, microparticles or compositions comprising microparticles are stable for several months and capable of being produced and/or maintained under sterile conditions.

In certain embodiments, microparticles or compositions comprising microparticles release one or more therapeutic agents over a period of several hours (e.g., 1 hour, 2 hours, 4 hours, 8 hours, 12 hours or 24 hours) to days (e.g., 1 day, 2 days, 3 days, 7 days, or 14 days) to months (e.g., 1 month, 2 months, 3 months, 6 months or 12 months). Release profiles may be characterized in terms of the initial rate, time for 50%, 90% or 100% drug release, or by appropriate kinetic models such as zero-order, first order, diffusion controlled (e.g., square-root of time, Higuchi model) kinetics, or by the number of distinct phases of release rate (e.g., monophasic, biphasic, or triphasic). The release profile may be characterized by the extent of its burst (initial) phase. The burst phase may result in little or large amounts of drug release and consequently microparticles may be defined as “low” or “high” burst systems. For example, low burst systems may release as little as about 30, 20, 10 or even 5 or 1% of the total amount loaded in the initial phase of release. High burst systems may release at least about 50, 60, 70 or even 100% of the total amount of drug in the burst phase. The duration of the burst phase is dependant on the overall intended duration of the release profile. For microparticles intended to release all of the loaded drug within hours, the burst phase may occur over several minutes (e.g., 1 to 30 minutes). For microparticles intended to release over several days, the burst phase may on the order of hours (e.g., 1 to 24 hours). For microparticles intended to release over several weeks, the burst phase may be from several hours to several days (e.g., 12 hours to 7 days). An exemplary release profile describing a microparticles release characteristics may be a low burst microsphere, releasing less than 10% in the first 24 hours, followed by a phase of approximately zero-order release and a gradual reduction in rate after 5 days, until all of the drug is depleted. Microparticles within the scope of this invention may have a widerange of release characteristics depending on the composition. For example, high load 5-fluororacil or mycophenolic acid microspheres made of a relatively hydrophilic polymer will have a high burst and release all of the drug with in several hours to a few days. Alternately, paclitaxel loaded poly(lactide) microspheres embedded in a PEG-based hydrogel scaffold, may release only a small fraction of the total dose over 5 days, with a very small burst phase.

In certain embodiments, microparticles or compositions comprising microparticles of the present invention are sterile. Many pharmaceuticals are manufactured to be sterile and this criterion is defined by the USP XXII <1211>. Sterilization in this embodiment may be accomplished by a number of means accepted in the industry and listed in the USP XXII <1211>, including without limitation autoclaving, dry heat, gas sterilization, ionizing radiation, and filtration. Sterilization may be maintained by what is termed aseptic processing, defined also in USP XXII <1211>. Acceptable gases used for gas sterilization include ethylene oxide. Acceptable radiation types used for ionizing radiation methods include gamma, for instance, from a cobalt 60 source and electron beam. A typical dose of gamma radiation is 2.5 MRad. Filtration may be accomplished using a filter with suitable pore size, such as 0.22 μm, and of a suitable material, such as TEFLON. In one aspect, when a polysaccharide such as HA is used as an excipient, sterilization should be by a method other than irradiation as HA tends to decompose upon exposure to γ radiation. Furthermore, a sterile composition may be achieved by using a combination of these sterilization methods and optionally aseptic techniques. In certain aspects of the invention comprising microparticles greater than 200 nm in diameter, a method of sterilization other than filtration should be used since the particles would not pass easily through a 0.22 μm filter. Since not all components of certain embodiments of the invention may be conveniently sterilized by a single method, sterilization may be accomplished by sterilizing components of the embodied invention in separate steps and combining the sterilized components into the embodied composition.

In certain embodiments, microparticles or compositions comprising microparticles of the present invention are contained in a container that allows them to be used for their intended purpose, i.e., as a pharmaceutical composition. Properties of the container that are important are a volume of empty space to allow for the addition of a constitution medium, such as water or other aqueous medium (e.g., saline), an acceptable light transmission characteristic in order to prevent light energy from damaging the composition in the container (refer to USP XXII <661>), an acceptable limit of extractables within the container material (refer to USP XXII), and an acceptable barrier capacity for moisture (refer to USP XXII <671>) or oxygen. In the case of oxygen penetration, this may be controlled by including in the container a positive pressure of an inert gas such as high purity nitrogen, or a noble gas such as argon.

Typical materials used to make containers for pharmaceuticals include USP Type I through III and Type NP glass (refer to USP XXII <661>), polyethylene, polyvinyl chloride, TEFLON, silicone, and gray-butyl rubber. For parenterals, USP Types I to III glass and polyethylene are preferred. In addition, a container may contain more than one chamber (e.g., a dual chamber syringe) to allow extrusion and mixing of separate solutions to generate a single bioactive composition. In one embodiment, microparticles dispersed in a carrier component (e.g., a polymer) may be in a first delivery chamber and a second carrier component (e.g., a buffer) may be in a second delivery chamber.

In certain embodiments, the compositions of the present invention are subjected to a process of lyophilization, comprising lyophilization of any of the compositions described above to create a lyophilized powder. In addition, compositions of the invention may be spray dried as described above. In one embodiment, the process further comprises reconstitution of the lyophilized powder with water or other aqueous media, such as benzyl alcohol-containing bacteriostatic water for injection, to create a reconstituted suspension of microparticles (Bacteriostatic Water for Injection, Abbott Laboratories, Abbott Park, Ill.).

In certain embodiments of the invention, compositions may be administered to a patient as a single dosage unit or form (e.g., stent, graft, film or gel), and the compositions may be administered as a plurality of dosage units (e.g., in aerosol form as a spray, or a cream dispensed from a multidose tube). For example, the high loading microparticle formulations may be sterilized and packaged in single-use, plastic laminated pouches or plastic tubes of dimensions selected to provide for routine, measured dispensing. In one example, the container may have dimensions anticipated to dispense 0.5 ml of the composition (e.g., a gel form) to a limited area of a target site or in a subject to treat or prevent a condition. A typical target, for example, is in the immediate vicinity of or within an arthritic joint, the site of a surgery, or within an aneurysm. In another aspect, the compositions of the instant invention may also be formulated for use in vitro, such as in experimental systems in the laboratory.

In another aspect, the present invention provides kits that include in one or more containers containing high drug loaded microparticles and optionally one or more containers containing a carrier and/or a scaffold. In certain embodiments, the kits further comprise a device for combining or mixing the microparticles with the carrier and/or the scaffold. One exemparly kit may include anti-microtubule agent loaded microparticles, a buffered aqueous carrier and a hydrogel scaffold. Another example of a kit includes: (a) a first container that contains a composition that includes one or more microparticles; (b) a second container that includes a buffer; (c) a third container that includes hydrogel forming components; and (d) a fourth container that includes having a buffer selected to result in crosslinking of the hydrogel forming components from the third container. The kit may be used by combining the contents of the first and third containers to form a first precursor composition and the contents of the second and fourth containers to form a second precursor composition. The final therapeutic composition is formed by combining the two precursor compositions, resulting in microparticles contained in a hydrogel scaffold. In yet another exemplary kit, separate containers are provided that contain: (a) high drug loaded microparticles; (b) a carrier solution; and (c) an absorbent scaffold (e.g., a pledget, gauze, a sponge, or a porous wafer). The kit may be used by combining the high drug loaded microparticles and the carrier solution to yield a suspension of microparticles. The suspension then may be absorbed into the scaffold by means such as dipping the absorbent material into the suspension or pouring the suspension into or onto the scaffold. A further exemplary kit may comprise a first container containing high drug loaded microparticles (e.g., microparticles containing higher than 50% lidocaine (w/w)), a second container comprising a carrier (e.g., a collagen composition), and a device allowing for mixing the microparticles and the carrier (e.g., a syringe). Another exemplary kit may comprise (i) a container containing high drug loaded microparticles and (ii) a scaffold.

### Clinical Applications

In one aspect, a method is provided for treating a disease or condition that comprising administering to a patient in need thereof (e.g., a mammal including human, horses and dogs) a therapeutically effective amount of a composition including microparticles having a high loading of an indicated drug as described herein. In certain embodiments, the method comprises delivering the therapeutic composition to a target site or confined space within the body.

As utilized herein, it should be understood that the terms “treat” or “treatment” refer to the therapeutic administration of a desired composition or compound in an amount and/or for a time sufficient to treat, inhibit, or prevent at least one aspect or marker of a disease, in a statistically or clinically significant manner. For example, the therapeutic efficacy of high loading microparticle composition according to the present invention is based on a successful clinical outcome and does not require 100% elimination of the symptoms associated with a disease such as an inflammatory disease (e.g., inflammatory arthritis, restenosis and surgical adhesions), infection, pain or a cancer. For example, achieving a drug level at the site of disease, which allows the patient to resolve or otherwise eradicate the symptoms, or allows the patient to have a better quality of life, is sufficient.

Compositions of the present invention may be administered by a variety of routes, depending on the condition targeted for treatment. In certain embodiments, the route of administration comprises intraarticular, intraperitoneal, topical, intravenous, intramuscular, subcutaneous, ocular, oral, rectal, into the urinary/genital tract, or to a surgically incised area such as a resection margin, incision, or anastomosis. For examples, treatment may be effected by local administration such as implantation into a dental pouch, an eye, a joint or a body passageway such as an artery or a duct. Administration may be regional, being not explicitly contained or confined to a space or structure in the body, but having limited systemic exposure of the drug, such as administration to a tumor resection site, administration by lavage, a subcutaneous implant, or exposure to the skin. Alternatively, the administration may be systemic, with the drug being distributed throughout the body such as by oral administration, intravenous infusion or intramuscular depot injection.

In order to further the understanding of the compositions and methods for their use, representative clinical applications are discussed in more detail below.

1. Inflammation

In certain embodiments, the present invention provides a method for treating an inflammationary condition comprising administering to a patient in need thereof an effective amount of microparticles or compositions comprising microparticles described herein. Such microparticles may comprise an anti-inflammatory agent, an analgesis, anti-neoplastic agent, anti-proliferative agent, anti-restenotic agnet, anti-infective agent, hemostatic agent, and/or anti-microtubule agent (e.g., paclitaxel or an analogue or derivative thereof).

An “inflammatory condition” as used herein refers to any of a number of conditions or diseases which are characterized by vascular changes: edema and infiltration of neutrophils (e.g., acute inflammatory reactions); infiltration of tissues by mononuclear cells; tissue destruction by inflammatory cells, connective tissue cells and their cellular products; and attempts at repair by connective tissue replacement (e.g., chronic inflammatory reactions). Representative examples of such conditions include many common medical conditions such as inflammatory arthritis, restenosis, adhesions (e.g., surgical adhesions), fibroproliferative opthalmic conditions, and tumors or excision sites.

In certain embodiments, methods are provided for treating or preventing inflammatory arthritis. Inflammatory arthritis refers to a number of inflammatory diseases that principally (although not solely) affect one or more joints. Representative examples of inflammatory arthritis include, but are not limited to, rheumatoid arthritis, systemic lupus erythematosus, systemic sclerosis (scleroderma), mixed connective tissue disease, Sjögren's syndrome, ankylosing spondylitis, Behçet's syndrome, sarcoidosis, and osteoarthritis all of which feature inflamed, painful joints as a prominent symptom. The methods for treatment comprise the step of administering to a patient a therapeutically effective amount of a high loaded drug microsphere containing an anti-inflammatory, anti-microtubule or analgesic agent, as described above. Within certain embodiments of the invention, microspheres may be administered directly to a joint by intra-articular injection in a liquid carrier or contained within a solid or semisolid matrix, for example a gel, hydrogel or polymer implant, or administered by another route, e.g., systemically, subcutaneously or orally.

An effective high drug loaded microsphere based therapy for inflammatory arthritis may accomplish one or more of the following: (i) decrease the severity of symptoms (pain, swelling and tenderness of affected joints; morning stiffness, weakness, fatigue, anorexia, weight loss); (ii) decrease the severity of clinical signs of the disease (thickening of the joint capsule, synovial hypertrophy, joint effusion, soft tissue contractures, decreased range of motion, ankylosis and fixed joint deformity); (iii) decrease the extra-articular manifestations of the disease (rheumatic nodules, vasculitis, pulmonary nodules, interstitial fibrosis, pericarditis, episcleritis, iritis, Felty's syndrome, osteoporosis); (iv) increase the frequency and duration of disease remission/symptom-free periods; (v) prevent fixed impairment and disability; and/or (vi) prevent/aftenuate chronic progression of the disease.

2. Adhesions

In certain embodiments, the present invention provides a method for preventing adhesions comprising administering to a patient in need thereof an effective amount of microparticles or compositions comprising microparticles described herein. Such microparticles may comprise anti-inflammatory agents, anti-proliferatives (including certain anticancer agents), anti-fibrotic agents (e.g., paclitaxel and analogues and derivatives thereof), or immunosuppressive agents. Microparticles may be incorporated into a carrier or scaffold for their administration in adhesion prevention. The carrier or scaffold may be a gel, hydrogel, film, woven fabric, spray, or solution. The carrier or scaffold may act to position the microparticles in addition to providing a barrier function.

Adhesion formation, a complex process in which bodily tissues that are normally separate grow together, is most commonly seen to occur as a result of surgical trauma. These post-operative adhesions occur in 60 to 90% of patients undergoing major gynecologic surgery and represent one of the most common causes of intestinal obstruction and infertility in the industrialized world. Other adhesion-treated complications include chronic pelvic pain, urethral obstruction and voiding dysfunction. Currently, preventative therapies, such inert surgical barriers made of hyaluronic acid or cellulose placed at the operative site at the time of surgery, are used to inhibit adhesion formation. Various modes of adhesion prevention have been examined, including (1) prevention of fibrin deposition, (2) reduction of local tissue inflammation, and (3) removal of fibrin deposits. Fibrin deposition is prevented through the use of physical barriers that are either mechanical or comprised of viscous solutions.

Utilizing the agents, compositions and methods provided herein, a wide variety of adhesions and complications of surgery can be treated or prevented. Adhesion formation or unwanted scar tissue accumulation and/or encapsulation complicate a variety of surgical procedures, such as open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule that compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies; infections such as fungal and mycobacterial).

A wide variety of animal models may be utilized in order to assess a particular therapeutic composition or treatment regimen. Briefly, peritoneal adhesions occur in animals as a result of severe inflicted damage, which usually involves two adjacent surfaces. Injuries may be mechanical, due to ischemia, or due to the introduction of foreign material. Mechanical injuries include crushing of the bowel (Choate et al., Arch. Surg. 88:249-254, 1964) and stripping or scrubbing away the outer layers of bowel wall (Gustavsson et al., Acta Chir. Scand. 109:327-333, 1955). Dividing major vessels to loops of the intestine induces ischemia (James et al., J. Path. Bact. 90:279-287, 1965). Foreign material that may be introduced into the area includes talcum (Green et al., Proc. Soc. Exp. Biol. Med. 133:544-550, 1970), gauze sponges (Lehman and Boys, Ann. Surg 111:427-435, 1940), toxic chemicals (Chancy, Arch. Surg. 60:1151-1153, 1950), bacteria (Moin et al., Am. J. Med. Sci. 250:675-679, 1965) and feces (Jackson, Surgery 44:507-518, 1958).

Additionally, adhesion prevention models may also be used, including the rabbit uterine horn model, which involves the abrasion of the rabbit uterus (Linsky et al., J. Reprod. Med. 32(1):17-20, 1987), the rabbit uterine horn; devascularization modification model, which involves abrasion and devascularization of the uterus (Wiseman et al., J. Invest Surg. 7:527-532, 1994); and the rabbit cecal sidewall model which involves the excision of a patch of parietal peritoneum plus the abrasion of the cecum (Wiseman and Johns, Fertil. Steril. Suppl: 25S, 1993).

3. Tumor

In certain embodiments, the present invention provides a method for preventing local recurrence of cancer by administering to a patient in need thereof high drug loaded microparticles or compositions comprising such microparticles at a tumor excision site in a therapeutically effective amount. In certain embodiments, the microparticles comprise an anti-tumor agent. In certain related embodiments, the present invention provides a method for treating cancer by administering to a patient in need thereof microparticles that comprise a high loading of an anti-cancer agent or a composition that comprises the microparticles in a therapeutically effective amount.

Local recurrence of malignancy following primary surgical excision of the mass remains a significant clinical problem. In one series of breast cancer patients who underwent lumpectomy of a primary breast tumor, almost ⅔ of the patients that presented with recurrent disease had local (i.e., tumor in the same breast) disease, while only ⅓ presented with metastatic disease. Other pathological studies have demonstrated that most local tumor recurrence occurs within a 2 cm margin of the primary resection margin. Therefore, treatments designed to address this problem are greatly needed. Local recurrence is also a significant problem in the surgical management of brain tumors. For example, within one embodiment of the invention, anti-microtubule compositions may be administered to the site of a neurological tumor subsequent to excision, such that recurrence of the brain tumor (benign or malignant) is inhibited. Briefly, the brain is highly functionally localized; i.e., each specific anatomical region is specialized to carry out a specific function. Therefore it is the location of brain tumor pathology that is often more important than the type. A relatively small lesion in a key area can be far more devastating than a much larger lesion in a less important area. Similarly, a lesion on the surface of the brain may be easy to resect surgically, while the same tumor located deep in the brain may not (one would have to cut through too many vital structures to reach it). Also, even benign tumors can be dangerous for several reasons: they may grow in a key area and cause significant damage; even though they would be cured by surgical resection this may not be possible; and finally, if left unchecked they can cause increased intracranial pressure. The skull is an enclosed space incapable of expansion. Therefore, if something is growing in one location, something else must be being compressed in another location—the result is increased pressure in the skull or increased intracranial pressure. If such a condition is left untreated, vital structures can be compressed, resulting in death. The incidence of CNS (central nervous system) malignancies is 8-16 per 100,000. The prognosis of primary malignancy of the brain is dismal, with a median survival of less than one year, even following surgical resection. These tumors, especially gliomas, are predominantly a local disease that recurs within 2 centimeters of the original focus of disease after surgical removal.

Representative examples of brain tumors which may be treated utilizing the compositions and methods described herein include glial tumors (such as anaplastic astrocytoma, glioblastoma multiform, pilocytic astrocytoma, oligodendroglioma, ependymoma, myxopapillary ependymoma, subependymoma, choroid plexus papilloma); neuron tumors (e.g., neuroblastoma, ganglioneuroblastoma, ganglioneuroma, and medulloblastoma); pineal gland tumors (e.g., pineoblastoma and pineocytoma); menigeal tumors (e.g., meningioma, meningeal hemangiopericytoma, meningeal sarcoma); tumors of nerve sheath cells (e.g., schwannoma (neurolemmoma) and neurofibroma); lymphomas (e.g., Hodgkin's and non-Hodgkin's lymphoma (including numerous subtypes, both primary and secondary); malformative tumors (e.g., craniopharyngioma, epidermoid cysts, dermoid cysts and colloid cysts); and metastatic tumors (which can be derived from virtually any tumor, the most common being from lung, breast, melanoma, kidney, and gastrointestinal tract tumors).

Within one embodiment of the invention, the compound or composition is administered directly to the tumor excision site (e.g., applied by painting, spraying, swabbing, brushing or otherwise coating the resection margins of the tumor with the microparticle composition(s)). Within particular embodiments of the invention, the treatment is applied to hepatic, colon, breast, bladder, nerological, ovarian, head and neck tumor resections. Alternately, the treatment may be applied to tumors after radiotherapy.

For the treatment of tumor resection margins, any anti-cancer agents selected for their specific activity in a given clinical application may be used. For example, breast cancer tumor resections may be treated with paclitaxel. For paclitaxel, a variety of embodiments are described for the management of local tumor recurrence. In one embodiment, 1-25 mg of paclitaxel is loaded into a microsphere at a loading of 70%, incorporated into a hyaluronic acid carrier and applied to the resection surface as a “paste”, “film”, or “gel” which releases the drug over a period of time such that the incidence of tumor recurrence is reduced. In another embodiment, the microparticles are incorporated into a gel or hydrogel comprising a polyether such as a polyethylene glycol or PLURONIC polymer. Optionally these polymers are cross-linked. During endoscopic procedures, 1-25 mg of paclitaxel contained in the microsphere is applied as a “spray”, via delivery ports in an endoscope, to the resection site. In another embodiment, an intraperitoneal surgical lavage fluid containing 10 to 250 mg paclitaxel in 70% loaded microparticles is administered at the time of, or immediately following, surgery. For this last embodiment, a fluid that has the added property of mucoadherence (i.e., adheres selectively to the mesenteric and peritoneal surfaces of the abdomen) would be preferred. Other appropriate anticancer agents may be used in their appropriate doses in a similar manner.

In certain embodiments, the treatment may be administered prior to tumor resection, or as a chemotherapy when no surgical treatment is possible. For example, rather than resection for example in the case of a diffuse, widespread peritoneal cancer, high drug loaded microparticles containing for example paclitaxel (or any other suitable agent) may be instilled into the peritoneum in a suspension, providing and efficacious drug concentration throughout the cavity.

4. Analgesia

In certain embodiments, the present invention provides a method for treating or preventing pain by administering to a patient in need thereof microparticles that comprise a high loading of an analgesia or a composition that comprises the microparticles in a therapeutically effective amount.

Pain is the most common symptomatic complaint among the general patient population. It may result from acute or chronic conditions and from a wide variety of underlying pathologies and as such treatments vary. Generally, pain is best treated by prevention (e.g., by elimination of its root cause); however, symptomatic therapies are also often required due to the often debilitating nature of pain.

Microparticles useful in treating or preventing pain may be fast or slow releasing depending on the precise nature of the pain. Microparticles may be administered locally, at the site of pain, if the drug's mechanism of action is on the peripheral nervous system or other locally occurring biochemistry, or may be administered (e.g., subcutaneously) so as to provide efficacious systemic concentrations for centrally acting agents. Analgesics that may be administered according to these methods include non-steroidal anti-inflammatories, non-narcotics (e.g., acetaminophen), and narcotic agents (e.g., codeine). Additionally, anticonvulsants such as phenyloin may be used for neuropathic pain, or amphetamines (e.g., dextroamphetamine) or antihistamines (e.g., hydroxyzine) may be used for somatic or visceral pain treatment. Topical anesthetics may also be used (e.g., lidcocaine), particularly for pain arising from a site on the surface of the body.

For topical application to sites of pain such as topical cuts, abrasions, incisions, and burns, an analgesic drug may be administered in a cream, ointment, spray, powder, or other suitable carrier having within it microparticles with a high loading of the drug. Additionally, a scaffold such as a bandage or patch holding the microspheres may be used. For injections such as subcutaneous injection, a dispersion of microspheres in a liquid carrier may be used.

5. Infection and Prophylaxis

In certain embodiments, the present invention provides a method for treating or preventing infection whereby microparticles with a high loading of an anti-infective agent (e.g., penicillin, cephalosporin, erythromycin, or a cipro drug, such as quinolone) or a composition comprising the microparticles is administered to a patient in need thereof in a therapeutically effective amount.

The infection may be caused by microorganisms including for example bacteria, yeasts, virus, worms, spirochetes and the like. Infection is a significant medical problem in both developed and third world countries. Continually new microbial pathogens and are identified, while some of the most common infections (e.g., pneumococcal pneumonia, Chlamydia, and HIV) continue to be a leading cause of death and morbidity worldwide. As a result, new therapies for these conditions are continually sought, including both novel antibiotic and antiviral drugs, and novel treatment regimes or drug delivery systems. Exemplary conditions which may be treated by this method are, without limitation, tuberculosis, which may be treated for example with microspheres having a high loading of rifampicin, delivered to the lungs (e.g., by inhalation); purulent burns treated for example with microspheres with a high loading of vancomycin contained in a cream carrier or within a dressing pad; streptococcal infections including purulent skin infections (streptococcal pyoderma) and necrotizing fasciitis (streptococcal gangrene), treated for example by intralesional injection or topical application of rapidly dissolving microspheres with a high load of drugs such as clindamycin and penicillin; intra-articular infections, treated with an injectable suspension of high drug loading microspheres; gastric tract infections of Helicobacter pylori or related ulcers, treated with orally administered microspheres containing a high loading of amoxicillin; systemic infections (e.g., sepsis or septic arthritis); orthopedic infection secondary to a spinal impant procedurel; osteopmyelitis using microspheres loaded with gentamycin contained in a carrier paste or dispersion which may include a wax, hydroxyapatite or mineral salt such as CaPO₄; periodontitis, treated with, for example, high drug loading microspheres containing cefazolin, placed within the periodontal pouch; topical infections treated with erythromycin or tetracycline; bacterial prostatitis treated by intraprostatic injection of microspheres containing for example ofloxacin; staphylococcus infections treated for example by the intraperitoneal implantation of microspheres having a high load of methicillin.

In addition to the treatment of infections, methods are provided for the prophylaxis of infection, particularly in cases where an increased risk of infection exists. Such a risk may be in immunocompromised patients receiving immune suppression drugs or chemotherapy agents, or having diseases which cause immunodeficiency. Exemplary applications of such prophylaxis include, without limitation, post surgical infection, infection following joint surgery in fracture repair, infection following oral/dental surgery or procedures, potential infection of patients receiving catheters, both vascular and urinary, particularly in-dwelling catheters, or potential infection of topical wounds or burns. In some of these applications, microparticles containing the efficacious antibiotic or anti-infective agent may be contained within or on a scaffold appropriate to the application. For example in post surgical infection, the scaffold may be a suture and in the case of catheter-caused infection prevention, the scaffold may be the catheter.

Additional methods for the treatment or prevention of infection are disclosed wherein high loading microspheres are administered in feeds, or as drug delivery systems to animals such as chickens, cattle, fish in aquaculture, other livestock or pets such as dogs and cats.

6. Implants and Surgical or Medical devices

In certain embodiments, the present invention provides methods for making implants and surgical or medical devices that comprise high drug loaded microspheres or a composition that comprises the microsphere.

A variety of implants, surgical devices or stents, may be coated with or otherwise constructed to contain and/or release certain embodiments of the high drug loading microparticles provided herein. Representative examples include cardiovascular devices (e.g., implantable venous catheters, venous ports, tunneled venous catheters, chronic infusion lines or ports, including hepatic artery infusion catheters, pacemaker wires, implantable defibrillators); neurologic/neurosurgical devices (e.g., ventricular peritoneal shunts, ventricular atrial shunts, nerve stimulator devices, dural patches and implants to prevent epidural fibrosis post-laminectomy, devices for continuous subarachnoid infusions); gastrointestinal devices (e.g., chronic indwelling catheters, feeding tubes, portosystemic shunts, shunts for ascites, peritoneal implants for drug delivery, peritoneal dialysis catheters, implantable meshes for hernias, suspensions or solid implants to prevent surgical adhesions, including meshes); genitourinary devices (e.g., uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy); opthalmologic implants (e.g., multino implants and other implants for neovascular glaucoma, drug eluting contact lenses for pterygiums, splints for failed dacrocystalrhinostomy, drug eluting contact lenses for corneal neovascularity, implants for diabetic retinopathy, drug eluting contact lenses for high risk corneal transplants); otolaryngology devices (e.g., ossicular implants, Eustachian tube splints or stents for glue ear or chronic otitis as an alternative to transtempanic drains); plastic surgery implants (e.g., prevention of fibrous contracture in response to gel- or saline-containing breast implants in the subpectoral or subglandular approaches or post-mastectomy, or chin implants), and orthopedic implants (e.g., cemented orthopedic prostheses).

Implants and other surgical or medical devices may act as scaffolds to be coated with (or otherwise adapted to contain or release) microparticle compositions of the present invention in a variety of manners, including for example: (a) by directly affixing to the implant or device a microparticle or composition (e.g., by electrostatic or chemical interaction by covalent or noncovalent means); (b) by coating the implant or device with a substance such as a hydrogel which will in turn absorb or contain the microparticle composition; (c) by coating or embedding the microparticles into a thread and interweaving the thread containing high drug loading microparticles into the implant or device; or (d) by inserting the implant or device into a sleeve or mesh which is comprised of or coated with microparticles of the present invention. Typically, it is desirable that the microparticle composition should firmly adhere to or be embedded in the implant or device during storage and at the time of insertion. The microparticle composition should also preferably not degrade during storage, prior to insertion, or when warmed to body temperature after insertion inside the body (if this is required). For vascular stents, in addition to the above properties, the composition should not render the stent thrombogenic (causing blood clots to form), or cause significant turbulence in blood flow (more than the stent itself would be expected to cause if it was uncoated).

7. Digestive Tract Diseases

In certain embodiments, the present invention provides a method for treating digestive tract diseases whereby microparticles with a high load of an anti-inflammatory agent or an anti-infective agent or a composition comprising the microparticles is administered to a patient in need thereof in a therapeutically effective amount.

For example, utilizing the compositions and methods provided herein, a wide variety of diseases of the bowel can be treated or prevented. Inflammatory bowel disease is a general term for a group of chronic inflammatory disorders of unknown etiology involving the gastrointestinal tract. Chronic IBD is divided into 2 groups: ulcerative colitis and Crohn's disease. In Western Europe and the United States, ulcerative colitis has an incidence of 6 to 8 cases per 100,000. Methods for treatment of these conditions may include oral administration of compositions that contain drugs that are clinically effective in treating these conditions. Anti-inflammatory agents, both steroidal and non-steroidal may be use, as can TNF-α inhibitors such as remicade. In certain cases, antibiotics may also be used, for instance in the case of certain ulcers in which H pylori is implicated. Alternatively, such agents may be administered rectally, by injection or to the surfaces of affected tissues in the course of a surgical procedure.

8. Surgical Procedures

High drug loading microparticles as well as their compositions may be utilized in a wide variety of surgical procedures. For example, within certain embodiments, an anti-cancer agent or composition (in the form of a high drug loading microparticle) may be utilized to coat or spray an area prior to removal of a tumor, in order to isolate normal surrounding tissues from malignant tissue, and/or to prevent the spread of disease to surrounding tissues. Within other aspects of the present invention, anti-cancer agents or compositions (e.g., in the form of a spray) may be delivered via endoscopic procedures in order to coat tumors, or inhibit disease in a desired locale. Within yet other aspects of the present invention, surgical meshes which have been coated with or adapted to release anti-cancer agents or compositions of the present invention may be utilized in any procedure wherein a surgical mesh might be utilized. For example, within one embodiment of the invention, a surgical mesh laden with an anti-cancer agent loaded microparticle (e.g., 70% w/w paclitaxel or cisplatin loaded PLGA microparticles) composition may be utilized during abdominal cancer resection surgery (e.g., subsequent to colon resection) to provide support to the structure, and to release an amount of the anti-cancer agent.

In certain embodiments, the high drug loading microparticle (e.g., those comprising hemostatic agents) may be administered during surgery to provide hemostasis to reduce or stop bleeding.

9. Chronic Inflammatory Diseases of the Respiratory Tract

In certain embodiments, the present invention provides a method for treating or preventing chronic inflammatory disease of the respiratory tract whereby microparticles with a high loading of an anti-inflammatory agent, an anti-microtubule agent or another effective agent or a composition comprising the microparticles is administered to a patient in need thereof in a therapeutically effective amount. Exemplary chronic inflammatory diseases of the respiratory tract that may be treated include asthma and chronic obstructive pulmonary disease (COPD). Within certain embodiments of the invention, the agents or compositions may be administered intranasally, systemically, by inhalation, topically (e.g., in the case of nasal polyps), or into the sinus cavities in order to achieve statistically significant clinical results.

10. Skin Diseases

In certain embodiments, the present invention provides a method for treating or preventing skin diseases whereby microparticles with high loading of a drug (such as an anti-inflammatory agent, an anti-infective agent, an anti-cancer agent, an anesthetic, or an analgestic) or a composition that comprises the microparticles is administered to a patient in need thereof in a therapeutically effective amount.

For example, within one embodiment of the invention, an inflammatory skin disease such as psoriasis or eczema may be treated or prevented by delivering to a site of inflammation (or a potential site of inflammation) high drug loading microparticle that inhibits microtubule function or other inflammatory or proliferative processes. Alternatively, topical cancers, such as Kaposi's sarcoma may be treated with microspheres containing a high load of an anticancer agent. In further examples of clinical applications, topical infections or burns may be treated with high loaded microparticles. For such treatments antibiotics or anti-infectives may be loaded into microparticles. Alternatively, anti-inflammatory agents, or topical anaesthetics or analgesics could be used for symptomatic relieve of pain or irritation. For such applications, microparticles of the present invention may be incorporated into a carrier such as an ointment, lotion or cream. Alternatively, a scaffold may be additionally employed, such as a patch or wound dressing, which is impregnated with high drug loading microparticles. In other embodiments, a suspension of microparticles may be injected intralesionally or subcutaneously beneath or adjacent to the lesion.

11. Restenosis

In certain embodiments, the present invention provides a method for treating or preventing restenosis whereby high drug loaded microparticles or a composition that comprises the microparticles is administered to a patient in need thereof in a therapeutically effective amount.

Restenosis is a form of chronic vascular injury leading to vessel wall thickening and loss of blood flow to the tissue supplied by the blood vessel. It occurs in response to vascular reconstructive procedures, including virtually any manipulation that attempts to relieve vessel obstructions, and is the major factor limiting the effectiveness of invasive treatments for vascular diseases.

Therapeutic agents that may be used for loading the microparticles include, but are not limited to, agents directed at treatment of endothelial loss, anti-platelet agents (e.g., aspirin), vasodilators (e.g., calcium channel blockers), antithrombotics (e.g., heparin), anti-inflammatory agents (e.g., steroids), agents which prevent vascular smooth muscle cell (VSMC) proliferation (e.g., colchicine), promoters of re-endothelialization (e.g., vascular endothelial growth factor), and heparin.

In certain embodiments, treatment may be achieved by incorporation of the microparticles into or onto medical devices used in related procedures, including stents, grafts, anastomotic closure devices, sealants and the like, as described above.

12. Fibrosis

In certain embodiments, the present invention provides a method for inhibiting fibrosis whereby microparticles that comprise an anti-fibrotic agent or a composition that comprises the microparticles is administered to a patient in need thereof in a therapeutically effective amount.

The clinical function of certain medical implants and devices may be dependent upon the devices being able to effectively maintain an anatomical, or surgically created, space or passageway. Unfortunately, many devices implanted in the body are subject to a “foreign body” response from the surrounding host tissues. In particular, injury to tubular anatomical structures (such as blood vessels, the gastrointestinal tract, the male and female reproductive tract, the urinary tract, sinuses, spinal nerve root canals, lacrimal ducts, Eustachian tubes, the auditory canal, and the respiratory tract) from surgery and/or injury created by the implantation of medical devices can lead to a well known clinical problem called “stenosis” (or narrowing). Stenosis occurs in response to trauma to the epithelial lining or the entire body tube during the procedure, including virtually any manipulation that attempts to relieve obstruction of the passageway, and is a major factor limiting the effectiveness of invasive treatments for a variety of diseases

In certain embodiments, microparticles that comprise anti-fibrotic agents or compositions that comprise the microparticles may be used to coat or otherwise attach to a medical device of which clinical functions may be adversely affected by fibrotic responses of a host to the device. Such medical devices include, but are not limited to, various intravascular implants (e.g., vascular graft or wrap, hemodialysis access, and implants that provides anatomotic connection), ventricular assist implants, prosthetic heart valve implants, inferior vena cava filter implants, peritoneal dialysis catheter implants, central nervous system shunts, intraocular lens, glaucoma drainage devices, penile implants, endothacheal tubes, tracheostomy tubes, gastrointestinal devices, spinal implants, pressure monitoring implants, tympanostomy tube implants, implantable nonvascular stents or tubes, central venous catheter implants, neurostimulators, cardiac rhythm management devices, other electrical devices (e.g., electrical leads), implantable sensors, implantable pumps, and soft tissue implants (e.g., breast, facial, chin, mandibular, lip, nasal, check, pectoral, buttocks, and autogenous tissue implants).

In certain related embodiments, microparticles that comprise anti-fibrotic agents, compositions comprising the microparticles or medical devices that comprises the microparticles or the compositions may be used to prevent surgical adhesions, treat or prevent inflammatory arthritis, treat hypertrophic scar or keloid, reduce or prevent cartilage loss, treat vascular disease (e.g., stenosis, restenosis, and atherosclerosis), or treat benign fibrotic hyperplasias.

In certain embodiments, the present invention provides a method for promoting fibrosis whereby microparticles that comprise a fibrosing agent or a composition that comprises the microparticles is administered to a patient in need thereof in a therapeutically effective amount.

The clinical performance of certain medical devices may also depend upon the devices being effectively anchored into the surrounding tissue to provide either structural support or to facilitate scarring and healing. Effective attachment of the device into the surrounding tissue, however, is not always readily achieved. One reason for ineffective attachment is that implantable medical devices generally are composed of materials that are highly biocompatible and designed to reduce the host tissue response. These materials (e.g., stainless steel, titanium based alloys, fluoropolymers, and ceramics) typically do not provide a good substrate for host tissue attachment and ingrowth during the scarring process. As a result of poor attachment between the device and the host tissue, devices can have a tendency to migrate within the vessel or tissue in which they are implanted.

In certain embodiments, microparticles that comprise fibrosing agents described herein and compositions comprising the microparticles may be used to coat or otherwise attach to a medical device intended to be present inside a host for a significant period of time. Such medical devices include, but are not limited to, intravascular devices (e.g., stents and stent grafts), spinal fusion devices, hernia mesh implants, vascular coil implants, soft palate implants, gastric restriction implants, suture-based endoluminal implants, electrostimulation implants, anal sphincters, urinary slings, fallopian tube implants, vas defenens implants, orthopedic implants, dental implants, joint implants, surgical films, septal occlusion patches, and endoluminal fasterners. The fibrosing agents promote fibrosis and in turn allow for better attachment between the devices and the tissue in the host surrounding the device.

In certain embodiments, microparticles that comprise fibrosing agents, compositions comprising the microparticles or medical devices that comprises the microparticles or the compositions may be used to treat vulnerable plaques, treat aneurysm, reduce perigraft leakage, treat shoulder injury, provide pulmonary sealing, treat or prevent aneurysm, treat fecal incontinence, provide hernia repair, treat obesity, treat gastroesophageal reflux disease (GERD), treat urinary incontinence, provide contraception, treat orthopedic conditions, or treat dental conditions.

13. Tissue Filling

In certain embodiments, the present invention provides a method for tissue filling whereby high drug loaded microparticles or a composition that comprises the microparticles is administered to a patient in need thereof in a therapeutically effective amount. The microparticles or the composition comprising the microparticles may be combined with a tissue filler before, concurrently, or after the tissue filler is implanted into a host. Alternatively, the microparticles or the composition comprising the microparticles may be combined with ingredients for forming a tissue filler to produce a drug loaded tissue filler. The resulting drug loaded tissue filler may then be implanted into a host. Exemplary drugs useful in tissue filling include anti-fibrotic agents (to prevent scarring or undesirable fibrosis between a tissue filler and the surrounding tissue), anti-infective agents (to prevent or reduce infection at the site of the tissue filler implantation), anti-inflammatory agents (to prevent or reduce inflammation due to the implantation of the tissue filler), and local anesthetics (to prevent or reduce pain associated with the implantation of the tissue filler).

Exemplary tissue fillers that may be combined with microparticles or compositions comprising microparticles of the present invention include, but are not limited to collagen or hyaluronic acid implants (e.g., CosmoDerm™, CosmoPlast™, Zyderm®, Zyplast®, and Hylaform®) and implants containing solid bulking materials such as hydroxyapatite, polymethylmethacrylate, polylactide-co-glycolide, or ceramic materials. Additional tissue fillers include soft tissue implants described above.

In certain embodiments, the tissue fillers in combination of microparticles or compositions comprising the microparticles of the present invention may be administered intradermally or subcutaneously into humans or other mammals to augment soft tissue, to repair tissue defects, to correct congenital anomalies, to correct cosmetic defects, and the like. Such defects or anomalies may be caused by aging, environmental exposure, weight loss, child bearing, surgery, diseases (e.g., acne and skin cancer), or combinations thereof. The defects or anomalies include, but are not limited to, frown lines, worry lines, wrinkles, crow's feet, marionette lines, stretch marks, and internal or external scars resulted from injury, wound, surgery, bites, cuts, or accidents. The tissue fillers in combination of microparticles or compositions of the present invention may also be injected into internal tissues to augment such tissues or treating diseases. For instance, they may be injected into the vocal cord, nose, and the tissues defining body sphincters (e.g., the lower esophageal sphincter, the diaphragm, the bladder sphincter or urethra) for augmenting or repairing such tissues and treating diseases such as gastroesophageal reflux disease, urinary incontinence (e.g., caused by bladder-neck hypermobility), or urinary reflux disease. In certain other embodiments, the tissue fillers in combination of microparticles or compositions comprising the microparticles of the present invention may also be used for repair or augmentation of hard tissues, such as bone, cartilage, connective tissues, and the like.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. The invention having been described, the following examples are intended to illustrate, and not limit, the invention.

### EXAMPLES

### Example 1

### Synthesis of Polymers for the Production of High Loading Microparticles

Polyester polymers were synthesized for use in the production of high drug loading (e.g., 50 to 90% w/w loading) microparticles. Polymers were produced by ring opening polymerization using the alcoholic initiators listed in Table 1 and monomers listed in Table 2.

TABLE 1
Initiators used for ring opening polymerization reactions.

| Initiator | Molecular Weight (g/mol) |
|---|---|
| Methoxypolyethylene glycol | 2000 |
| Methoxypolyethylene glycol | 5000 |
| Salicylic acid | 138 |
| 1-Octadecanol | 270 |
| 1-Octanol | 130 |

TABLE 2
Monomers used for ring opening polymerization reactions.

| Initiator | Molecular Weight (g/mol) |
|---|---|
| Glycolide | 116 |
| L-Lactide | 144 |
| DL-Lactide | 144 |
| ε-Caprolactone | 114 |
| δ-Valerolactone | 100 |
| δ-Decanolactone | 170 |

The reagents were charged into a 50 ml round bottom flask reaction vessel in sufficient quantity to give a total mass (batch size) of 5, 10, 50 or 100 g, and in ratios appropriate for the desired molecular weight (Target MW) and in the case of copolymers, monomer ratio. The mass of initiator required was determined using the following equation:

Mass of Initiator (g)=MW of Initiator (g/mol)×Target Polymer MW (g/mol)×Batch Size (g)

The mass of monomer required was determined using the following equation:

Mass of Monomer (g)=Batch Size (g)−Mass of Initiator (g)

For copolymers (e.g., of glycolide and DL-lactide), two monomers were combined in weight ratios that were then listed in the polymer's name, along with the monomers and initiators used and the target molecular weight. For example “Salicylic acid-PDLLA/GA(75/25) (MW=3000)” denotes a polymer synthesized using salicylic acid as the initiator, and DL-lactide and glycolide in a 75:25 weight ratio, and a target molecular weight of 3000 g/mol.

After charging the reaction vessel a TEFLON-coated stir bar was added and the vessel transferred to an oil bath previously equilibrated to 140° C. The oil bath was a glass beaker with heavy mineral oil, heated on a Corning combination hot-plate/stirrer, equipped with a Dyna-Sense Mkl On/Off Digital Temperature Controller. The Corning hot-plate was set to a heat setting of “6” and the temperature controller set to 140° C. (284° F.). The system equilibrated after approximately 15 minutes. The flask was submerged to the neck and stirred with a stir setting of “6”. After at least 10 minutes, the reagents had melted to form a homogeneous liquid at which point 0.5% w/w stannous octoate was added to catalyze the polymerization. Constant stirring was maintained for several minutes and the reaction was maintained with stirring at 140° C. for approximately 6 hours.

After polymerization was completed, the product was poured from the reaction vessel onto glass or stainless steel plates or trays and allowed to solidify. The solid polymer was broken into pieces using a spatula and transferred to glass bottles with TEFLON-lined caps for storage. Some polymers were stored at 2-8° C. and some were frozen to approximately −20° C. Exemplary batches of polymers produced by this method are summarized in Table 3.

TABLE 3
Polymers Synthesized by Ring-Opening Polymerization

| | Batch Size | Molecular Weight(s) (MW) |
|---|---|---|
| Polymer | (g) | Synthesized (g/mol) |
| MePEG2000-PDLLA | 50 | 2857, 3636, 4444, 5000, 6667, |
| | | 10000, 40000 |
| MePEG2000-PLLA | 50 | 3333, 4000, 5000, 10000, 20000 |
| MePEG2000-PGA | 50 | 3333, 4000 |
| MePEG2000-PCL | 50 | 2500, 3333, 5000, 10000 |
| MePEG2000-Poly(δ- | 50 | 3333 |
| Decanolactone) | | |
| MePEG2000-Poly(δ- | 50 | 3333 |
| Valerolactone) | | |
| MePEG5000-PDLLA | | 3076, 8333, 16667, 25000 |
| MePEG5000-PLLA | | 100000 |
| C18-PLLA | 10 | 2000 |
| SA-PLGA(75/25) | 5 | 1000, 2000, 3000, 4000, 5000 |
| SA-PLGA(25/75) | 5 | 1000, 2000, 3000, 4000, 5000 |
| SA-PLGA(75/25) | 5 | 1000, 2000, 3000, 4000, 5000 |

Abbreviations:
MePEG2000 = Methoxypolyethylene glycol MW = 2000;
MePEG5000 = Methoxypolyethylene glycol MW = 5000;
SA = salicylic acid;
C18 = 1-Octadecanol;
C8 = 1-Octanol
PCL = Poly(ε-caprolactone);
PDLLA = Poly(DL-Lactide);
PLLA = Poly(L-lactide);
PLGA = Poly(DL-lactide-co-glycolide);
PGA = Poly(glycolide).

### Example 2

### Evaluation of the Solubility of Polymers

Certain polymers, prepared according to the method of Example 1, were evaluated for their solubility in exemplary production solvents, namely water and dichloromethane. Approximately 5% w/v polymer (accurately weighed) was dispersed into deionized water in a 50 ml beaker, covered with tin foil. A 5% w/v polymer dispersion in dichloromethane was made by combining 1 g of polymer and 20 ml of dichloromethane in a 20 ml glass vial with a polypropylene-lined screw cap lid. To each dispersion, a TEFLON-lined stir bar was added and the mixtures stirred using a VARIO-MAG Multi-stir Plate (Daytona Beach, Fla.) on its lowest setting for at least 3.5 hours. The physical appearance of each mixture was used to grade the polymer's solubility in either water or dichloromethane. Clear solutions indicated solubility; hazy or cloudy mixtures were called partly soluble and mixtures having particles were: considered to have poor solubility.

Polymers with good solubility in a processing solvent and poor solubility in water may be considered good candidates for use in preparing microparticles by the solvent evaporation (O/W) method (Example 3). For polymers with an opposite solubility profile, a w/o method may be preferred. As well, this test may be used in screening solvents that may be useful in forming microparticles using a spray drying technique (Example 5).

The method is suitable for the evaluation of any number of other production solvents, such as tetrahydrofuran, toluene, chloroform, acetone, alcohols and dimethylacetamide. The dissolution time may be increased to several hours if desired since the ultimate result is sought to be evaluated rather than the kinetics of dissolution. For example higher molecular weight polymers in solvents such as tetrahydrofuran may require longer dissolution times. Table 4 summarizes the results for a number of polymers.

TABLE 4
Solubility of polymers in water and dichloromethane.

| | Molecular | | |
|---|---|---|---|
| | Weight | Solubility in | Solubility in |
| Polymer | (g/mol) | Water | Dichloromethane |
| MePEG2000-PLLA | 3333 | Partly Soluble | Soluble |
| | 5000 | Partly Soluble | Soluble |
| | 10000 | Soluble | Partly Soluble |
| | 20000 | Partly Soluble | Not Tested |
| | 40000 | Partly Soluble | Not Tested |
| MePEG5000-PLLA | 8333 | Partly Soluble | Soluble |
| | 100000 | Not Soluble | Not Tested |
| MePEG2000-PDLLA | 2857 | Soluble | Soluble |
| | 3333 | Partly Soluble | Not Tested |
| | 3636 | Soluble | Soluble |
| | 4000 | Soluble | Soluble |
| | 4444 | Soluble | Not Tested |
| | 5000 | Partly Soluble | Soluble |
| | 6667 | Partly Soluble | Soluble |
| | 10000 | Not Soluble | Soluble |
| MePEG5000-PDLLA | 7692 | Partly Soluble | Partly Soluble |
| | 8333 | Partly Soluble | Not Tested |
| | 16667 | Partly Soluble | Not Tested |
| | 25000 | Partly Soluble | Not Tested |
| MePEG2000-PCL | 2500 | Partly Soluble | Soluble |
| | 3333 | Partly Soluble | Soluble |
| | 5000 | Partly Soluble | Not Tested |
| MePEG2000-PGA | 3333 | Partly Soluble | Partly Soluble |
| MePEG2000-Poly(δ- | 3333 | Not Soluble | Not Tested |
| Decanolactone) | | | |
| MePEG2000-Poly(δ-X | 3333 | Partly Soluble | Not Tested |
| Decanolactone) | | | |

Abbreviations:
MePEG2000 = Methoxypolyethylene glycol MW = 2000;
MePEG5000 = Methoxypolyethylene glycol MW = 5000;
PCL = Poly(ε-caprolactone);
PDLLA = Poly(DL-Lactide);
PLLA = Poly(L-lactide);
PGA = Poly(glycolide).

### Example 3

### Preparation of Microparticles by a Solvent Evaporation Method

High drug loading (i.e., 50% to 90% loading) microparticles were prepared by a solvent evaporation method as follows. A 500 ml quantity of an aqueous stabilizer solution (10% poly(vinyl alcohol) (PVA) (87-89% hydrolyzed, MW 13,000-23,000)) was prepared by mixing 50 g PVA and 500 ml deionized water in a 1000-ml glass bottle. A TEFLON-coated stir bar was added and the PVA was dissolved with stirring and low heat using a Corning stirrer/hot plate (heat setting 4, stir setting 6). After all the PVA had dissolved, the solution was cooled down at ambient conditions for at least 3 hours. A 100 ml aliquot of the 10% PVA solution was poured into a 1000 ml glass beaker, to be used in microparticle production. The beaker was anchored with double-sided tape to the floor of a fumehood, to provide stability.

Aliquots of drug and polymer were weighed into a 50 ml beaker and then dissolved in 20 ml of dichloromethane. The masses of each depended on the batch size (either 1.0 g or 0.5 g) and theoretical drug loading (% w/w), and were calculated using the following equations:

Mass of drug (g)=Batch Size (g)×Theoretical Drug Loading (% w/w)

Mass of polymer (g)=Batch Size (g)−Mass of Drug (g)

Several batches using different drug-polymer combinations were prepared and are summarized in Table 4.

TABLE 4
High drug loading microparticles made by the solvent evaporation
method.

| | % Drug | |
|---|---|---|
| Drug Type | Loading | Polymer |
| Lidocaine | 70 | PLLA |
| (S)-(+)-6-methoxy- | 70 | PLLA (MW = 2000) (Polysciences Inc.) |
| α-methyl-2- | | |
| napthaleneacetic | | |
| acid (Naproxen) | | |
| Hydrocortisone 21- | 70 | PLLA (MW = 2000) (Polysciences Inc.) |
| caprylate | | |
| Lidocaine | 80 | PLLA (MW = 2000) (Polysciences Inc.) |
| Erythromycin | 70 | PLLA (MW = 2000) (Polysciences Inc.) |
| Paclitaxel | 70 | PLLA (MW = 2000) (Polysciences Inc.) |
| Paclitaxel | 70 | 60/40 PLGA |
| Paclitaxel | 70 | 50/50 PLGA |
| Paclitaxel | 90 | 50/50 PLGA |
| Paclitaxel | 70 | MePEG750-PDLLA (MW = 3750) |
| Paclitaxel | 70 and 80 | C8-PLLA (MW = 1200) |
| Paclitaxel | 70 and 80 | C18-PLLA (MW = 1200) |
| Paclitaxel | 70 | MePEG5000-PLLA (MW = 50000) |
| Paclitaxel | 70 | PLLA (MW = 2000) (Polysciences Inc.) |

Abbreviations:
MePEG750 = Methoxypolyethylene glycol MW = 750;
MePEG5000 = Methoxypolyethylene glycol MW = 5000;
C18 = 1-Octadecanol;
C8 = 1-Octanol;
PDLLA = Poly(DL-Lactide);
PLLA = Poly(L-lactide);
PLGA = Poly(DL-lactide-co-glycolide).

The organic phase was stirred to dissolve the drug and polymer in dichloromethane and then it was added to the 100 ml aqueous phase as follows.

The aqueous phase (PVA solution) in the 1000-ml beaker was stirred at a rate of 1000 or 2000 rpm using an overhead Dyna-Mix (Fisher Scientific) stirring motor and a Troemer 150¼×12″ 2″ propeller blade. The blade's stir rate was determined using a Monarch strobe light (Nova Strobe DA 115) set at 1000 flashes per minute. Using a Pasteur pipette, the organic phase was added drop-wise to the stirring PVA solution. The resulting dispersion was stirred for 3 hours then the contents of beaker were poured in four fractions into 50-ml Falcon tubes. The Falcon tubes were centrifuged (Beckman J6-HC centrifuge) for 10 minutes at 2500 rpm and 20° C. The supernatants of each tube were discarded and the pellets resuspended and pooled in a single 50-ml blue Falcon tube using deionized water. The pooled microparticle product was washed as follows. The tube containing the pooled pellets was filled with deionized water to contain 50 ml and was then vortexed for 15 seconds (Fisher Vortex Genie 2, lot# 12-812, setting 8), before being centrifuged again for 10 minutes at 2500 rpm and 20° C. The washing step was repeated for a total of three washes. After washing the pellet was resuspended in 5 to 7 ml of deionized water and the dispersion frozen by submersion of the bottom of the Falcon tube in a 50 ml mixture of acetone:dry ice for 10 minutes. The Falcon tube was removed from the acetone:dry ice mixture and the frozen dispersion freeze dried on a side port of a Stoppering Tray Dryer (Labconco) attached to a Freeze Dryer (Labconco, Freezone 8) for at least 48 hours. Freeze dryer conditions were a trap temperature of −45° C. and vacuum pressure of less than 0.133 mbar. Freeze drying resulted in the production of a white powder comprised of microparticles.

The products of this method were observed by optical microscopy at up to 1000× magnification to evaluate the sphericity of microparticles, their size and tendency to aggregate. Lidocaine microspheres (70 and 80% w/w) had low yields of microspheres with a diameter of 0.5 to 2 μm. Phenyloin (70% w/w 5,5-diphenylhydantoin) microspheres were of a similar size, with some of irregular shape and a small number of crystals and aggregates. Hydrocortisone caprylate (70% w/w) microspheres were in the 0.5 to 5 μm size range. Larger (approximately 5 μm) particles tended to have irregular shapes. Erythromycin stearate microspheres were approximately 1 μm in diameter. No evidence of drug crystallization or particle aggregation was observed.

### Example 4

### Evaluation of Various Polymeric Stabilizers in the Aqueous Phase Used in the Solvent Evaporation Method

The effectiveness of various polymeric stabilizers in the aqueous phase used the production of microparticles using the method described in Example 3. The evaluation used various polymers (listed in Table 3) in place of the PVA used in Example 3. For the evaluation, 200 ml quantities of aqueous stabilizer were prepared and 100 ml aliquots were used to prepare microparticles from 2000 g/mol PLLA. Stabilizer solutions were dissolved using a Corning stirrer/hot plate (stir setting 6). PVA required both stirring and heat to dissolve (Corning stirrer/hot plate—heat setting 4, stir setting 6), whereas other polymers dissolved at ambient conditions After dissolution, the viscosity of each aqueous phase was determined using a Brookfield Programmable DVIII+Rheometer (Model RVDV-III+CP) and CP40 (CPE40) spindle (viscosity range 1.7-32,700 cP). The rheometer was standardized with a CANNON Certified Viscosity Standard (S600) (lot no. 98301). The 10% PVA (87-89% hydrolyzed, MW 13,000-23,000) stabilizer solution's (i.e., the “standard” stabilizer solution's) viscosity was determined first. Other polymeric stabilizer solutions' viscosities were determined and compared to the standard stabilizer solution's viscosity. Solutions with a viscosity similar to that of the standard solution (e.g., not different by more than 5 cP) were accepted while those with different viscosities were prepared at higher or lower concentrations if the viscosity was lower or higher than that of the standard solution, respectively. Concentrations were adjusted using the assumption that viscosity and concentration were linearly related. After adjusting the concentrations, each polymeric stabilizer was used in the preparation of microparticles and the product evaluated. Table 6 summarizes the polymeric stabilizer solutions tested for viscosity and the final concentrations of each polymer used in the production of microparticles.

TABLE 6
Polymeric stabilizers evaluated in the solvent evaporation method.

| | | Concentration Used in the Aquueous |
|---|---|---|
| | Viscosity | Phase in the Solvent Evaporation |
| % (w/v) Polymeric Stabilizer Solution | (cP) | Method |
| 10% PVA (87-89% hydrolyzed, MW 13,000-23,000) | 12.62 | 10% |
| 5% PVA (99+% hydrolyzed, MW 124,000-186,000) | 54.7 | Diluted to 1.25%. |
| 10% PVA (98% hydrolyzed, MW 13,000-23,000) | 14.4 | 10% |
| 5% Dextran Sulfate (MW 500,000) | 23.0 | Diluted to 2.5%. |
| 10% Polyvinylpyrollidone (PVP) (MW 55,000) | 11.6 | 10% |
| 1% Carbopol | 19.5 | Diluted to 0.67%. |
| 10% Polaxamer 188 (MW 7,680-9,510) | 6.0 | Prepared again at 20%. |

Abbreviations in the table.
PVA = poly(vinyl alcohol)

After preparing microparticles, the product was observed by optical microscopy to ascertain its quality. The presence of microspheres (spherical microparticles), crystals (non-incorporated drug) and aggregation of microparticles was noted and is summarized in Table 7.

TABLE 7
Product evaluation of microspheres prepared.

| % w/v Stabilizer Solution | Paclitaxel Loading | Polymer | Observations |
|---|---|---|---|
| 1.25% PVA (99+% | 70 | 85/15 PLGA | Some microspheres, some |
| hydrolyzed, MW 124,000-186,000) | 70 | PLLA (MW 2000) | irregular microparticles with |
| | | | crystals. |
| 10% PVA (98% | Control | 85/15 PLGA | Some microspheres and |
| hydrolyzed, MW 13,000-23,000) | | | microparticles |
| | 70 | 85/15 PLGA | Irregular microparticles |
| | 70 | PLLA (MW 2000) | Some microspheres, some |
| | | | irregular microparticles |
| 10% PVA (87-89% | Control | PLLA (MW = 2000) | Microspheres |
| hydrolyzed, MW 13,000-23,000) | 70 | PLLA (MW 1000) | Microspheres |
| | 80 | PLLA (MW 1200) | Microspheres |
| | 80 | PLLA (MW 2000) | Microspheres |
| | 70 | PCL (MW 10,000) | Aggregated mass |
| | 70 | PCL (MW 80,000) | Aggregated mass |
| | 70 | MePEG750/PDLLA | Microspheres |
| | 70 | MePEG5000/PLLA | Microspheres |
| | 70 | PDLLA (MW 2000) | Microspheres |
| | 70 | 60/40 PLGA (MW | Microspheres with some |
| | | 2500) | aggregation |
| | | 50/50 PLGA (MW | Microspheres and crystals |
| | | 7900) | |
| | 70 | 50/50 PLGA (MW | Microspheres |
| | | 7900) | |
| 1% PVA (87-89% | Control | 50/50 PLGA | Microspheres |
| hydrolyzed, MW 13,000-23,000) | 10 | 50/50 PLGA | Microspheres |
| | 90 | 50/50 PLGA | Microspheres |
| 2.5% Dextran Sulfate | 70 | 85/15 PLGA | Microspheres |
| (MW 500,000) | 70 | PLLA (MW 2000) | Some microspheres, |
| | | | irregular microparticles and |
| | | | crystals |
| 10% PVP (MW 55,000) | Control | 85/15 PLGA | Some microspheres, |
| | | | irregular microparticles and |
| | | | crystals |
| | 70 | 85/15 PLGA | Some microspheres |
| 0.67% Carbopol | 70 | 85/15 PLGA | Aggregated mass |
| | 70 | PLLA (MW 2000) | Microspheres and crystals |
| 20% Polaxamer 188 | 70 | 85/15 PLGA | Few microspheres |
| (MW 7,680-9,510) | 70 | PLLA (MW 2000) | Aggregated mass |

Abbreviations:
MePEG750 = Methoxypolyethylene glycol MW = 750;
MePEG5000 = Methoxypolyethylene glycol MW = 5000;
PDLLA = Poly(DL-lactide);
PLLA = Poly(L-lactide);
PCL = Poly (caprolactone);
PLGA = Poly(DL-lactide-co-glycolide);
PVP = Polyvinyl pyrrolidone

### Example 5

### Preparation of Microparticles by a Spray Drying Method

Microparticles having a high percentage of drug loading (i.e., 50% to 90% loading) were prepared by a spray drying method as follows. Aliquots of drug, polymer and dichloromethane were weighed into a 250 ml round bottom flask and then dissolved in 20 ml of dichloromethane. The quantity of each depended on the batch size (either 1.0 g or 1.5 g) and theoretical drug loading (% w/w), and were calculated using the following equations:

Mass of drug (g)=Batch Size (g)×Theoretical Drug Loading (% w/w)

Mass of polymer (g)=Batch Size (g)−Mass of Drug (g)

Volume of dichloromethane (ml)=Batch Size (g)×100 ml/g

Several drugs were used to produce high drug loading microparticles with 2000 g/mol PLLA (Polysciences Inc.) (Table 8).

TABLE 8
Compositions of high drug loading microparticles.

| Theoretical Drug | | |
|---|---|---|
| Loading (% w/w) | Drug Type | Visual Appearance of Product |
| 70 | Lidocaine | Some aggregation, <1 to 8 μm |
| | | microspheres |
| 80 | Lidocaine | <1 to approximately 10 μm |
| | | microspheres |
| 70 | (S)-(+)-6-methoxy-α-methyl-2- | approximately 1-5 μm microspheres, |
| | napthaleneacetic acid (Naproxen) | some 1 μm microparticles and |
| | | aggregation |
| 70 | Hydrocortisone 21-caprylate | approximately 15 μm microspheres |
| | | with aggregation and smaller |
| | | microparticles |
| 70 | Mycophenolic acid | microparticles |
| 70 | Erythromycin | approximately 1 to 5 μm |
| | | microparticles with aggregation and |
| | | some crystals |
| 70 | Paclitaxel | microparticles with no crystals of |
| | | drug |
| 70 | 5,5-diphenylhydantoin (phenytoin) | approximately 1 to 5 μm |
| | | microparticles and microspheres with |
| | | no aggregation |

A stir bar was placed into the 250-ml round bottom flask, and the mixture was stirred using a Corning stirrer/hot plate (heat setting off, stir setting 6) until all polymer and drug was dissolved in the dichloromethane. The Buchi Mini Spray Dryer (type B-191) equipment was rinsed with acetone, allowed to dry, and set up. The unit was set with the parameters listed in Table 9.

TABLE 9
Spray drying parameters used to prepare high drug loading
microparticles.

| Parameter | Parameter Setting |
|---|---|
| Inlet preset ° C. | 48 |
| Aspirator % | 100 |
| Pump % | 50 |

Before spray drying microspheres, the spray dryer temperature was allowed to stabilize until the “inlet actual ° C.” was the same as the “inlet preset ° C.”. This was done by aspirating the unit with heat until the “inlet actual ° C.” read 47, and then pumping the unit through with dichloromethane for approximately 5 minutes until the “inlet actual ° C.” read 48. Once the inlet temperature was stable, the contents of the 250-ml round bottom flask were spray dried. The spray dried microparticles were collected in a glass screw capped vial.

### Example 6

### Evaluation of Microparticle Total Drug Content Using UV Spectroscopy

Methods: The measured drug loading in microparticles made by methods described in Examples 3 and 5 was determined for several drugs by UV spectroscopy as follows. For each drug, a characteristic wavelength at which the drug absorbs was determined from a 0.5% w/v drug (in dichloromethane) solution using an HP 8453 UV Spectrophotometer and Agilent Chemstation software. The wavelength analyzed was 200 to 400 nm. For drug solutions yielding absorbance values greater than 3 AU, solutions were diluted 5 to 25 fold and reanalyzed to yield spectra with distinct patterns and signal strength that did not overload the instrument. The characteristic wavelength was selected from each drug's spectral pattern as one with strong UV absorptivity. Control polymer solutions were used as blanks for analysis of microparticles. The concentration of polymer was selected in the blank to approximate the anticipated polymer concentration in samples. For example, to prepare 10 ml of 0.5% w/v polymer solution, 5 mg of polymer was dissolved in 10 ml of dichloromethane. The UV spectrum of the polymer solution was observed between 200 and 400 nm to ensure no interfering absorbance characteristics existed. Using the Agilent Chemstation software, the UV spectra of the polymer and drug to be analyzed were overlaid to determine the optimal wavelength for analysis. Optimal wavelengths (Table 10) typically showed a drug peak with an absorbance between 0.5 and 1.5, and no polymer peak. Using five standard solutions of the drug, a standard curve was constructed for absorbance at the selected wavelength. Standard concentrations were selected to yield a maximum absorbance of approximately 1.5 AU.

The drug loading level of microparticles prepared by the methods described in Examples 3 and 5 were determined by dissolving a sample of microparticles to a concentration at which the theoretical drug loading would be within the standard curve range. Test solutions were prepared in volumetric glassware by dissolving an accurately weighed quantity of microparticles in dichloromethane, with stirring at ambient temperature until clear solutions were formed. Clear solutions were analyzed in the same manner as standard solutions.

Results: Microparticles containing the following drugs were analyzed: lidocaine, naproxen, erythromycin stearate, and hydrocortisone 21-caprylate. All of the microparticles tested were made with PLLA (MW=2000) from Polysciences Inc. Standard curves for each are described by the regression parameters of the standard curves, summarized in Table 10. The measured loading and encapsulation efficiency of high drug loading microparticles are summarized in Table 11.

TABLE 10
Parameters describing the measurement of standard drug solutions.

| | Standard | Analysis | | |
|---|---|---|---|---|
| | Solution | Wave- | | |
| | Concentration | length | | |
| Drug | Range (% w/v) | (nm) | Linear Equation | R² |
| Lidocaine | 0.002-0.006 | 231 | y = 208x + 0.046 | 0.9998 |
| Naproxen | 0.0001-0.0005 | 234 | y = 2877x + | 0.9968 |
| | | | 0.0137 | |
| Erythromycin | 1.8-3.0 | 294 | y = 0.542x − | 0.9872 |
| Stearate | | | 0.476 | |
| Hydrocortisone | 0.001-0.005 | 239 | y = 387x + | 0.9997 |
| 21-caprylate | | | 0.0285 | |

TABLE 11
Measured loading of drugs in microparticles.

| | Measured | |
|---|---|---|
| | Loading (% | Encapsulation |
| High Drug Loading Microspheres | w/w) | Efficiency (%) |
| 70%-Erythromycin Stearate PLLA microspheres (spray | 78 | 112* |
| dried) | | |
| 70%-Lidocaine PLLA microspheres (spray dried) | 70 | 99 |
| 80%-Lidocaine PLLA microspheres (spray dried) | 70 | 87 |
| 70%-Hydrocortisone 21-caprylate PLLA microspheres | 97 | 138 |
| (solvent evaporation) | | |
| 70%-Hydrocortisone 21-caprylate PLLA microspheres (spray | 64 | 92 |
| dried) | | |
| 70%-Naproxen PLLA microspheres (solvent evaporation) | 71 | 101 |
| 70%-Naproxen PLLA microspheres (spray dried) | 85 | 122 |

*Encapsulation Efficiency values greater than 100% are due to greater efficiency of incorporation of the drug than the excipient.

### Example 7

### Evaluation of Microparticle Total Paclitaxel Content Using UV HPLC

Method: The total content of paclitaxel in microparticles made by the methods described in Examples 3 and 5 was determined using an Agilent 1100 HPLC system equipped with a diode array UV detector and Chemstation software. Samples were prepared to have a target paclitaxel concentration between 200-1000 μg/ml paclitaxel. For example, 70% w/w loaded microparticles were dissolved at 10 mg in 10 ml acetonitrile to yield a target concentration of 700 μg/ml. The test solution was injected (10 μl) onto a PFP Curasil column (150×4.6 mm×5 μm) and eluted using gradient mobile phase. The gradient parameters were 30% v/v acetonitrile in water for 20 minutes, increasing to 50% v/v acetonitrile over 3 minutes, increasing to 90% v/v acetonitrile over 5 minutes, decreasing to 30% v/v acetonitrile over 0.5 minutes and running at 30% v/v acetonitrile for 1.5 minutes thereafter. The flow rate was 2 ml/min. A total UV spectrum was obtained using the DAD detector and the absorbance at 227 nm was used to quantify paclitaxel concentrations in samples.

Results: Total content data collected by this method is summarized in Table 12. Microspheres made by the solvent evaporation method showed lower encapsulation efficiencies than those prepared by the spray drying method. However, the efficiency of both methods was sufficient to produce high loading microspheres with paclitaxel using a number of polymers. The table also shows the total content data for four lots of microspheres made with traditional (lower) contents of 10-50% w/w. These data show that the solvent evaporation method incorporates drug with comparable efficiency at all loadings from 10 to 90% w/w (77-105% encapsulation efficiency). No trend in theoretical loading was observed in the encapsulation efficiencies calculated.

TABLE 12
Total content of paclitaxel loaded microspheres.

| | | | | |
|---|---|---|---|---|
| | Theoretical | Measured | | |
| | Loading | Loading | Standard | Encapsulation |
| Polymer | (% w/w) | (% w/w) | Deviation | Efficiency (%) |

Microspheres Made by the Solvent Evaporation Method

| | | | | |
|---|---|---|---|---|
| 50/50 PLGA 0.15 dL/g | 10 | 7.7 | 0.3 | 77 |
| 50/50 PLGA 0.15 dL/g | 20 | 13.6 | 0.3 | 68 |
| PLLA MW = 2000 | 40 | 42.0 | 0.7 | 105 |
| PLLA MW = 2000 | 50 | 45.2 | 2.0 | 90 |
| PLLA | 60 | 50.9 | (n = 2) | 85 |
| PLLA | 70 | 70.7 | 1.4 | 101 |
| PLLA (Birmingham Polymers 99 dL/g) | 70 | 41.7 | 0.2 | 60 |
| C8-PLLA (MW = 1000) | 70 | 45.2 | 0.7 | 65 |
| C8-PLLA (MW = 1200) | 70 | 42.3 | 0.2 | 60 |
| C18-PLLA (MW = 1200) | 70 | 65.6 | 1.2 | 94 |
| 10/90 MePEG5000-PLLA | 70 | 61.4 | 2.1 | 88 |
| PDLLA (MW = 2000) | 70 | 57.1 | 1.3 | 82 |
| PLLA (MW = 2000), Polysciences, Inc. | 80 | 54.2 | 2.6 | 68 |
| C8-PLLA (MW = 1200) | 80 | 63.6 | 1.7 | 80 |
| C18-PLLA (MW = 1200) | 80 | 73.2 | 1.6 | 92 |
| PLLA (MW = 2000), Polysciences, Inc. | 90 | 76.5 | 1.8 | 85 |

Spray Dried Microspheres

| | | | | |
|---|---|---|---|---|
| Spray dried MePEG5000-PDLLA | 10 | 10.0 | 0.2 | 100 |
| PLLA (MW = 2000) | 64 | 63.1 | 0.3 | 99 |
| 60/40 MePEG5000-PDLLA | 70 | 70.8 | 0.6 | 101* |
| 65/35 MePEG5000-PDLLA | 70 | 70.6 | 1.6 | 101 |
| 60/40 PLGA (MW = 2500) | 70 | 68.2 | 6.4 | 97 |
| 20/80 MePEG750/PDLLA | 70 | 73.7 | 2.1 | 105 |
| Spray dried 50/50 PLGA MW = 7000 | 70 | 70.9 | 0.9 | 101 |
| 60/40 MePEG2000-PDLLA | 70 | 71.9 | 0.6 | 103 |
| Spray dried 50/50 PLGA MW = 7000 | 70 | 59.5 | 1.5 | 85 |
| 60/40 PLGA (MW = 2500) | 70 | 82.1 | 1.1 | 117 |
| Spray dried 50/50 PLGA MW = 7000 | 70 | 61.9 | 0.2 | 88 |
| Spray dried 50/50 PLGA MW = 7000 | 90 | 92.0 | 0.7 | 102 |

*Encapsulation Efficiency values greater than 100% are due to greater efficiency of incorporation of the drug than the excipient.

### Example 8

### Particle Size Analysis of Microparticles by Laser Diffraction

Particle size of microparticles made by the methods in Examples 3 and 5 was determined using a Malvern Mastersizer2000 equipped with a Hydro2000S sampling unit and version 5.1 software. Samples were prepared by mixing microspheres and about 5 ml of deionized water in a 50-ml Blue Falcon tube. The amount of sample required varied with the microsphere type. Microsphere solutions were sonicated for at least 15 minutes using a VWR Scientific Aquasonic (model 50T) sonicator. The resulting dispersions were white or opaque. Dispersions containing clumps of particulates were sonicated for a further 5 to 10 minutes. The following measurement parameters were used for analysis:

TABLE 13
Spray drying process parameters.

| Parameter | Setting |
|---|---|
| Sample material name | “test” |
| | (refractive index = 1.9, |
| | absorption = 0.05) |
| Dispersant name | Water |
| | (refractive index = 1.33) |
| Model | General Purpose |
| Obscuration Limits | Default (10% to 20%) |
| Stir rate | 1995 rpm |
| All other parameters | Default setting |

Prior to each analysis, the Hydro2000S sampling unit was cleaned by filling and emptying the unit with deionized water at least 3 times. After being cleaned, the background was measured. Then, using a Pasteur pipette, sample was transferred dropwise into the Hydro2000S until the minimum obscuration limit was reached. The Hydro2000S Was used to sonicate (at a maximum setting) the sample solution for approximately 2 minutes. Sonication was stopped and the particle size of the sample measured. Resulting weighted residuals were observed to ensure they were less than 1%.

Results: Table 14 lists the particle size data for microparticles tested.

TABLE 14
Particle size of microparticles.

| | Method of | | |
|---|---|---|---|
| | Preparation | | Weighted |
| | (Polymeric | d(0.5) | Residual |
| Microsphere Description | stabilizer used) | (μm) | (%) |
| 70% erythromycin stearate in | SE (PVA 10%) | 10.4 | 0.953 |
| PLLA | | | |
| 70% lidocaine in PLLA | SD | 12.5 | 0.463 |
| 80% lidocaine in PLLA | SD | 10.9 | 0.490 |
| 70% hydrocortisone 21-caprylate | SE (PVA 10%) | 4.7 | 0.746 |
| in PLLA | | | |
| 70% hydrocortisone 21-caprylate | SD | 36.3 | 0.601 |
| in PLLA | | | |
| 70% naproxen in PLLA | SE(PVA 10%) | 7.8 | 0.705 |
| 70% naproxen in PLLA | SD | 16.2 | 2.928 |
| 70% paclitaxel in PLGA | SE(Carbopol | 97.4 | 2.084 |
| | 0.67%) | | |
| 70% paclitaxel in PLGA | SE (Dextran | 21.4 | 0.834 |
| | 2.5%) | | |
| 70% paclitaxel in PLGA | SE (PVA 10%) | 48.3 | 1.906 |
| 70% paclitaxel in PLGA | SE (PVP 10%) | 33.2 | 1.170 |

Abbreviations:
PLLA = Poly(L-lactide)
PLGA = Poly(DL-lactide-co-glycolide)
PVP = Polyvinylpyrrolidone
PVA = Poly(vinyl alcohol)
SE = solvent evaporation (made according to Example 3)
SD = spray drying (made according to Example 5)

Particle size distribution is represented in Table 15 by “d(0.5)”. This number refers to the size that 50% of all particles measured fall below. For example, “d(0.5)=4.7 μm” means that 50% of particles in the sample fall under 4.7 μm. Despite much sonication, many results reflect a degree of aggregation. The d(0.5) values did not all correlate with particle size estimates made by optical microscopy (400×).

### Example 9

### In Vitro Drug Release Properties of High-Load Paclitaxel Microsphere Formulations

Method: Microparticles tested in this manner were made by the methods of Examples 3 and 5. Release study experiments were conducted using replicates of 2-5 mg (accurately weighed) microspheres placed in 15 ml Kimax tubes with TEFLON-lined lids and 15 ml of release medium (0.02 M phosphate buffered saline (PBS) (pH=7.4), with X % albumin). Tubes were incubated at 37° C. rotating at 30 RPM on a 10 ° incline. At sampling intervals, tubes were centrifuged for 10 minutes at 2500 rpm to pellet microspheres. A 10 ml aliquot of the supernatant was sampled and replaced with 10 ml fresh release medium. Paclitaxel was extracted from the supernatant by solid phase extraction using a Rapidtrace T system with DSC₁₈ (Supelco) 3 ml cartridges and 2 ml acetonitrile to elute the drug from the cartridge over 40 seconds. The eluant was dried under N2 gas using a Turbovap™ drier for 50 minutes at 35° C. and 5-15 psi. The residue containing paclitaxel was reconstituted in 1 ml of 85% v/v acetonitrile in water with vortexing for about 30 seconds. Samples were then analyzed by HPLC.

HPLC Method: Samples were analyzed using an Agilent HPLC system with Chemstation software and UV detection at 254 nm. The injection volume was 10 μl onto a C18 column with a mobile phase of 60/40 v/v acetonitrile/water flowing at 1 ml/min. The run time was 10 minutes.

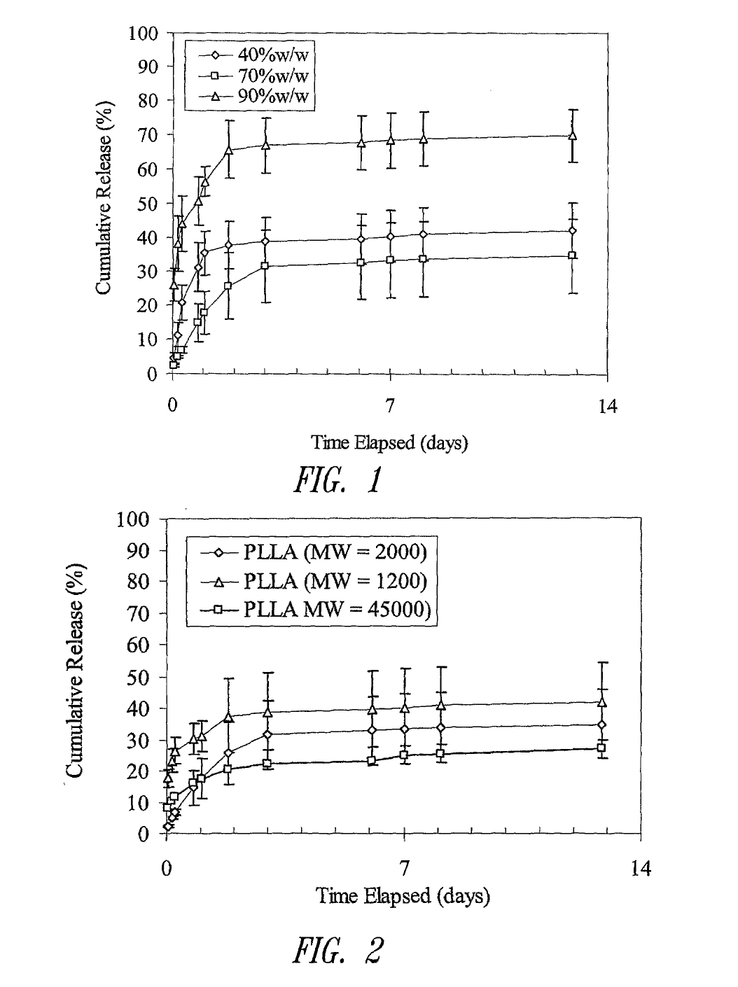

Results: Microspheres made with PLLA (MW=2000) were prepared having loadings of 40, 70, and 90% w/w paclitaxel contents. Using this method a release profile over 15 days was obtained, shown in FIG. 1. FIG. 2 shows the release profile for 70% paclitaxel loaded PLLA 1200, 2000, and 45,000 microparticles.

### Example 10

### Dissolution Characteristics of High-Drug Loaded Microsphere Formulations

Methods: The dissolution characteristics of high-drug loaded microspheres having 70% w/w paclitaxel in various polymers were determined as follows. Aliquots of microspheres (25 mg) were weighed into 60 ml glass jars with sealable lids which were modified include a 0.45 μm membrane having a cross-sectional area of about 9.6 cm² per jar. To each jar, a TEFLON coated stir bar was added, the jars filled with deionized water and the jars sealed. Jars with microspheres were placed in a water bath having about a 13 L capacity, filled with water. The water in the bath was circulated so that fresh water was exchange into it at a rate of 2 ml/min. Beneath the water bath a magnetic multi-stirrer was situated, having 15 stirring pads, allowing up to 15 samples to be analyzed simultaneously. Samples were stirred at 100-300 rpm. At weekly intervals, samples were removed and centrifuged to pellet all solids. The solids and a small amount of the supernatant (about 1-2 ml) were transferred to serum bottles and freeze dried in a Labconco Freeze drier, removing all but trace water. The resulting solid was analyzed for paclitaxel content using the method described in Example 6.

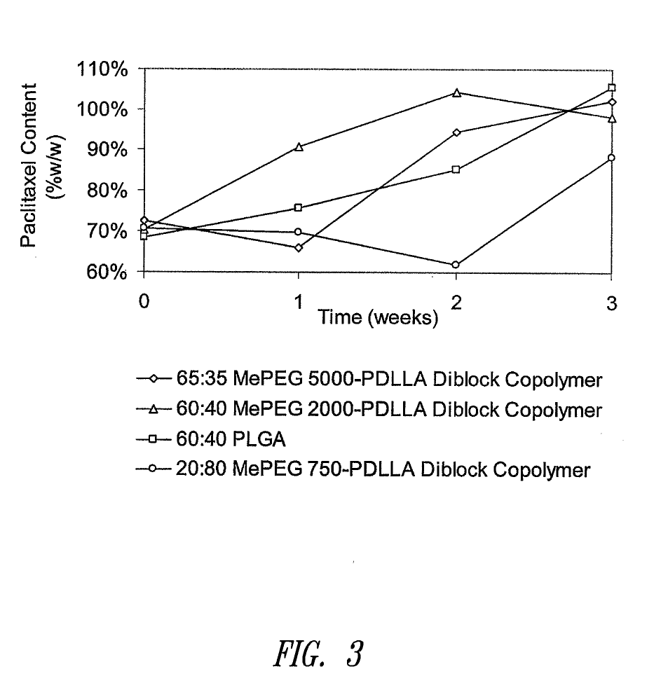

Results: After the first week, all samples lost between 50 and 80% of their total mass indicating significant dissolution of total mass over this time period. Over the following two weeks mass loss continued at a slower pace and not due to variability and testing only single replicates, no trends in weight loss over time were apparent. FIG. 3 shows the change in paclitaxel content by weight in samples over three weeks. The increase in paclitaxel content over three weeks showed that the polymers tend to dissolve more rapidly than the drug from the paclitaxel microparticles, so that the remaining solids become enriched with drug. The more hydrophobic polymers (60:40 PLGA and 20:80 MePEG750:PDLLA diblock) tended to show the slowest dissolution of polymer, resulting in a slower increase in paclitaxel content. After three weeks, three of the four samples were about 100% w/w paclitaxel, suggesting that substantially all of the polymer had dissolved, leaving only paclitaxel.

### Example 11

### High Loading Paclitaxel Microspheres Contained in an Hyaluronic Acid Gel Carrier

Preparation of the Gel Carrier: a Hyaluronic Acid (Ha) Gel Suitable for use as a carrier for high drug loading microparticles was prepared as follows. Hyaluronic acid (1 MDa HA, Genzyme, Cambridge, Mass.) (40 mg) was weighed into a tared 10 ml serum vial. To the vial was added 2 ml of sterile saline solution. A TEFLON-coated stir bar was added and the serum vial sealed with a gray butyl rubber septum and aluminum crimp seal. The mixture was allowed to stir on a magnetic stirrer (Corning) for several minutes to disperse the HA particles and initiate dissolution. The serum vial was vented with a 19 gauge needle and transferred to an autoclave. The mixture was heated to 121° C. for 15 minutes at 15 atm. After the autoclaving cycle was complete the serum vial was allowed to cool to ambient condition. The result was a homogeneous gel containing 20 mg/ml HA in saline suitable for in vivo administration.

Incorporation of high load microparticles: A microparticle formulation containing a theoretical loading of 70% w/w paclitaxel and an encapsulation efficiency of >95% in 2000 g/mol MW poly(L-lactide) (PLLA) was prepared according to Example 3A 6.4 mg aliquot of microparticles was weighed into a tared 10 ml serum vial and sealed with a gray butyl rubber stopper and an aluminum crimp seal. The vial was exposed to 2.5 MRad of γ irradiation using a Co-60 source at MDS Nordion (Location). After irradiation, the microparticles were constituted in 3 ml of sterile saline with vortexing (Vortex Genie) for several minutes. After a visually homogeneous suspension was achieved, a 2 ml aliquot was withdrawn from the vial into a 3 ml syringe and the aliquot transferred to a vial of HA gel. The mixture was stirred for at least 30 minutes on a magnetic stirrer (Corning) to form a homogeneous suspension of paclitaxel loaded microparticles with a theoretical loading of 1.5 mg/ml paclitaxel and 10 mg/ml HA.

### Example 12

### Assessment of Intra-Articular Biocompatibility High Drug Loaded Microparticles in a Polysaccharide Gel Carrier

Biocompatibility of paclitaxel given to guinea pigs by intra-articular injection may be assessed as follows. Paclitaxel was incorporated into the test article to form a hydrogel by means such as those described in Example 11. A 100 μl aliquot was administered by intraarticular injection into the right knee of a healthy male Hartley guinea pig aged at least 6 weeks. After injection, guinea pigs were housed 5 to a cage with free access to food and water. One week after injection, the animals were assessed for swelling, sacrificed, and the knee exposed for visual examination. Visual evidence of swelling or tissue irritation (fluid, vascularization) indicated an incompatibility of the formulation. Absence of these indicators indicated a positive result. Paclitaxel was loaded into a non-polysaccharide micellar carrier and used in this assay of biocompatibility. The results indicated that a 7.5 mg/ml dose of paclitaxel in the micellar carrier was not biocompatible, illiciting swelling and a tissue response, whereas a 1.5 mg/ml dose of paclitaxel in the micellar carrier was compatible, with no evidence of swelling or tissue response upon post-mortum examination.

The biodistribution of paclitaxel in the joint delivered using high dose microspheres may be determined by intra-articular administration in a similar manner. High drug loading microparticles contained in a hydrogel carrier are prepared according to the method of Example 11. A 100 μl aliquot is administered to guinea pigs according to the method of this example. After 4 or 24 hours, the guinea pigs are euthanized and joint tissues harvested. Tissues that may be harvested include cartilage, ligaments, fatty tissue, and synovium. Paclitaxel content may be measured in tissue by extraction and analysis by HPLC as described above.

### Example 13

### Intraperitoneal Administration of Microspheres in Saline to Prevent Tumor Cell Seeding

Microspheres with a high loading of an anti-cancer agent such as paclitaxel may be used to treat cancer such as intraperitoneal carcinomatosis which may arise as a result of tumor cells seeding the peritoneal cavity. The efficacy of 70% w/w paclitaxel loaded microspheres may be evaluated using the model established by Demetrick et al (Am J Surg 1997(173) 403-6) as follows. Tumor cells (e.g., 9 Lglioblastoma cells) sensitive to the drug are cultured in minimum essential medium with 10% fetal calf serum and 1% gentamicin. After incubation the cells are washed with phosphate buffered saline (PBS) (pH=7.4) and a 5% trypsin-EDTA solution. Cells are suspended in PBS without calcium at a concentration of 2 million cells/ml. Male Wistar rats weight 500 g are anaesthetized with atropine and Innovar and maintained on 3% halothane. Each rate receives a <=1 cm midline incision into the peritoneum, through which 1 ml of cell suspension is administered. Rats are immediately treated with a dose of paclitaxel loaded microspheres in saline, or control (saline alone). The dose may be in the range of 25-75 mg. In the current state of the art, a dose of 30 mg paclitaxel in 100 mg microspheres was efficacious. Thus, this new treatment improves the therapy by reducing the total biomaterial (excipient) load by up to a factor of three, with the potential for more rapid drug release than was observed in current state of the art.

After administering the tumor cells and the treatment, the incision is closed with a buried running suture and rats are allowed to recover, eating and drinking freely for a period of two weeks. At this time, the rats are sacrificed and the peritoneal cavity examined grossly and histologically for signs of cancer growth.

Using this same protocol other formulations may be tested, including microparticles in a carrier such as paclitaxel in a polysaccharide gel, made according to the method of Example 11.

### Example 14

### Preparation of High Load Microparticles in a Hydrogel Forming Carrier

High drug loading microparticles (e.g., microparticles containing up to 70% w/w paclitaxel) may be demonstrated to be efficacious in treating a cancerous tumor when administered in a hydrogel forming matrix as follows.

Formulation Preparation: A hydrogel forming formulation is prepared as follows. A “premix” is made by combining 40 mg each of pentaerythritol poly(ethylene glycol) ether tetra-succinimidyl glutarate and pentaerythritol poly(ethylene glycol) ether tetra-thiol into a 3 ml plastic syringe, weighing and transferring components in a dry argon atmosphere. The premix is constituted by adding 0.4 ml of a pH 9 sodium carbonate buffer. To a second 1 cc syringe 0.4 ml of 6.3 mM hydrochloric acid is added. In a third 1 cc syringe 20 mg of 70% w/w paclitaxel loaded microparticles are weighed and transferred. Immediately after constitution of the premix the 3 ml syringe is connected to the 1 ml syringe containing microparticles, by means of a male-male luer lok junction. The contents of both are combined by passing them back and forth between the two syringe barrels a minimum of twenty times. The combined liquid is transferred to the 1 cc syringe and it is disconnected from the luer lok junction. Both 1 cc syringes (one containing hydrochloric acid and one containing premix, pH 9.7 buffer and microparticles) are attached to a Micromedics, Inc. two syringe blending connector with cannula tip. The formulation is now ready for injection and is to be used within 10 minutes of its preparation.

### Example 15

### Preparation of Hydroxypropylcellulose Film Scaffolds Containing High Drug Loading Paclitaxel-Loaded Microparticles

Non-crosslinked films: Five grams of ethyl cellulose and hydroxypropyl cellulose (or other cellulose) with a ratio from 100:0 to 0:100 are dissolved in 100 ml of acetone in a glass jar having a screw-cap TEFLON-lined lid. Then 5-500 mg of microparticles (1-10 μm in diameter) having a theoretical loading of 70% w/w paclitaxel in 50 k g/mol PLLA are dispersed in the acetone solution with stirring of the mixture using a TEFLON-coated stir bar on a magnetic stirrer (Corning) for 5 minutes on a high setting. The dispersion is cast onto a release liner using a stainless steel casting knife with 40 mil opening. The dried cellulose film is obtained after the evaporation of acetone. The samples are further dried in vacuum oven overnight.

Crosslinked films: Five grams of ethyl cellulose and hydropropyl cellulose (or other cellulose) with a ratio from 100:0 to 0:100 are dissolved in 95 ml of acetone. Then 5-500 mg of paclitaxel are added and completely dissolved in the acetone solution. Then 4 ml of acetic acid solution (5%) was added into the solution to make the above solution pH around 2 to 3. Also, 1 ml of 5% glutaraldehyde solution is added into the above solution. The cellulose/acetone/paclitaxel solution is cast onto the release liner using a casting knife with 40 mil opening. The dried cellulose film is obtained after the evaporation of acetone. The samples are further dried in vacuum oven overnight.

### Example 16

### Incorporation of 70% w/w Loaded Microparticles into Topical Formulations

Two formulation types were prepared, an ointment and a cream. A 1% w/w lidocaine ointment was prepared as follows. A 10 mg aliquot of 70% w/w lidocaine microspheres was placed on a glass slab. To it 100 mg of petrolatum was added and the components mixed by levigation using a flat metal spatula blade for about 1 minute. After mixing, an additional 600 mg of petrolatum was added and mixed by further levigation for about 3 minutes. The result was an ointment having 7 mg lidocaine in 700 mg, or 1% w/w loading.

A 1% w/w hydrocortisone cream was prepared as follows. A cream base (Glaxal) was used as were 65% loaded hydrocortisone acetate microparticles. A 10 mg aliquot of microparticles and 650 mg of cream based were combined by levigation in a manner similar to that used for the lidocaine ointment.

This method is suitable for the incorporation of any number of pharmaceutically acceptable topical vehicles having at least the viscosity of a cream or ointment. Any number of high drug loading microparticles may be used, containing a variety of drugs.

### Example 17

### Efficacy of High Loading Microparticles in a Polysaccharide Matrix Assessed in a Rat Caecal-Sidewall Abrasion Model of Surgical Adhesions

Sprague Dawley rats are prepared for surgery by anesthetic induction with 5% halothane in an enclosed chamber. Animals are transferred to the surgical table, and anesthesia maintained by nose cone on halothane throughout the procedure and Buprenorphen 0.035 mg/kg is injected intramuscularly. The abdomen is shaved, sterilized, draped and entered via a midline incision. The caecum is lifted from the abdomen and placed on sterile gauze dampened with saline. Dorsal and ventral aspects of the caecum are scraped a total of 45 times over the terminal 1.5 cm using a #10 scalpel blade, held at a 45° angle. Blade angle and pressure are controlled to produce punctuated bleeding, while avoiding severe tissue damage or tearing. The left side of the abdominal cavity is retracted and everted to expose a section of the peritoneal wall nearest the natural resting caecal location. The exposed superficial layer of muscle (transverses abdominis) is then excised over an area of 1.0×1.5 cm². Excision includes portions of the underlying internal oblique muscle, leaving behind some intact and some torn fibers from the second layer. Minor local bleeding is tamponaded until controlled. The formulations containing a high drug loaded microparticle, for example those from Examples 11, 14 and 15, are deployed at the wounded areas, on the abraded sidewall, between the caecum and sidewall. The abraded caecum is then positioned over the sidewall wound and sutured at four points immediately beyond the dorsal corners of the wound edge. The large intestine is replaced in a natural orientation continuous with the caecum. The abdominal incision is then closed in two layers with 4-0 silk sutures. Healthy subjects are followed for one week, and then euthanized by lethal injection for post mortem examination to score. Severity of post-surgical adhesions is scored by independently assessing the tenacity and extent of adhesions at the site of caecal-sidewall abrasion, at the edges of the abraded site, and by evaluating the extent of intestinal attachments to the exposed caecum. Adhesions are scored on a scale of 0-4 with increasing severity and tenacity.

### Example 18

### High Load Microparticles in an Injectable Implant Useful as a Filling Agent

High load microparticles may be incorporated into a gel formulation that is suitable for use as a dermal filler. Such a formulation may contain collagen. Collagen may be obtained from a bovine source or from a human source (being the patient, cultured from the patient source or from another human (autolagous)). Collagen may be found in approved products such as ZYDERM. Collagen may also be obtained from commercial sources in a form suitable for use in medical products.

Alternatively, collagen may be obtained as follows: Collagen is obtained from rabbit skin, defatted, lyophilized and ground at low temperature (e.g. using a cryomill) to produce a fine powder. A suspension of the powdered skin in prepared by adding the powdered material to a 0.5 M acetic acid solution such that the skin concentration is 5 g dry wt skin/l. The suspension is cooled to 10° C. A freshly prepared pepsin solution (0.5 g in 10 ml 0.01 N HCl) is added to the skin suspension and the mixture was incubated for 5 days at 10° C. with occasional stirring. Following the enzymatic treatment, the pepsin in the mixture was denatured by adding 5 ml Tris base and adjusting the pH to 7.0 with 3 N NaOH at 4° C. 30 g NaCl is stirred into the mixture to keep the collagen in solution. After 4 hours, the mixture is centrifuged at 30,000 g for 30 minutes to remove the precipitated pepsin.

The enzymatically treated collagen is precipitated from the supernatant liquid by adding an additional 140 g NaCl. The solution is stirred and allowed to stand for 4 hours at 4° C. The precipitated collagen is centrifuged out at 30,000 g for 30 minutes. The resulting collagen pellet is resuspended in 200 ml deionized water. 0.5 N acetic acid is added to bring the final volume to one liter. The collagen is precipitated from this solution by adding 50 g NaCl, allowing the solution to stand for 5 hours at 4° C. and centrifuging at 30,000 g for 30 minutes.

The collagen pellet is resuspended in 200 ml distilled water, transferred into sterilized dialysis tubing and dialysed for 72 hours against 50 volumes 1 N acetic acid. The collagen is then dialysed for 24 hours against 50 volumes 0.001 N acetic acid with the solution being changed 3 times during this period. The dialysed solution is then concentrated by placing the dialysis tube on sterile absorbant towels in a laminar-flow bacteriologic barrier until the concentration reached 12-15 mg collagen/ml solution. The concentrated solution is then dialysed against 50 volumes 0.001 N acetic acid for 24 hours. The collagen solution is then stored in sterile vials at 4° C.

Immediately prior to use a buffered salt solution (NaCl 2.5 mM/l, NaHPO4 0.1 mM/l, pH 7.4) is added at 4° C. to the collagen solution in a volume:volume ratio of 10:1 (collagen:buffer), and the buffered concentrate is transferred to a chilled (4° C.) syringe.

Other filler materials may also be used to form a matrix for incorporation of lidocaine high load microparticles. These materials may be used to form injectable implants. They include: fibril, a gelatin powder compound that is mixed with a patient's own blood and is injected to plump up the skin (similar to injectable collagen); and GORTEX, a thread-like material that is implanted beneath the skin to add soft-tissue support. These materials suspended into an injection vehicle may be combined with the microparticles.

Incorporation of high load microparticles into a dermal filling material: A microparticle formulation containing a theoretical loading of 70% w/w lidocaine and an encapsulation efficiency of >95% in poly(L-lactide) (PLLA) was prepared according to Example 3. An aliquot of microparticles is weighed into a tared 10 ml serum vial and sealed with a gray butyl rubber stopper and an aluminum crimp seal. The microspheres may be sterilized. An aliquot of dermal filling material (e.g., collagen solution), described above is added to the vial and the contents are blended by, for example, stirring, vortexing or other agitation.

### Example 19

### Preparation of a Two Component Microsphere Kit

40 mg of the freeze-dried microsphere batimistat material is weighed into a capped 1 mL syringe. The plunger is replaced and the syringe is sealed in a plastic pouch using a heat sealer. The sample is sterilized using 2.5 Mrad γ-ray irradiation. Just prior to application, the plastic pouch containing the sterilized freeze-dried material is opened and connected to a dual syringe connector. A syringe containing 2 mL 3.5% bovine collagen (95% type 1 and 5% Type III) is attached to the remaining end of the dual syringe connector. The plunger of the syringe containing the collagen material is pushed in order to transfer the collagen material into the syringe containing the microsphere material. The material is passed from one syringe to the other until a homogeneous dispersion is obtained. The material is then transferred into the syringe that originally contained the collagen. This syringe is disconnected from the connector and a 30-gauge needle is connected to the syringe. The material is now ready for application.

From the foregoing, it is appreciated that, although specific embodiments of the invention have been described herein for purposes of illustration, various modifications may be made without deviating from the spirit and scope of the invention. Accordingly, the invention is not limited except as by the appended claims.

1. A composition comprising a microparticle wherein the microparticle comprises a polymer and a drug, and wherein the drug is present in the microparticle at a concentration of greater than 75% (weight of drug/weight of microparticle).

2. The composition of claim 1 wherein the drug is present in the microparticle at a concentration of greater than 80% (weight of drug/weight of microparticle).

3. The composition of claim 1 wherein the drug is present in the microparticle at a concentration of greater than 90% (weight of drug/weight of microparticle).

4. The composition of claim 1 wherein the polymer is a synthetic polymer.

5. The composition of claim 4 wherein the synthetic polymer comprises a polyester.

6. The composition of claim 4 wherein the polyester comprises the residues of one or more of the monomers selected from lactide, lactic acid, glycolide, glycolic acid, e-caprolactone, γ-caprolactone, hydroxyvaleric acid, hydroxybutyric acid, β-butyrolactone, γ-butyrolactone, gamma-valerolactone, γ-decanolactone, d-decanolactone, trimethylene carbonate, 1,4-dioxane-2-one and 1,5-dioxepan-2one.

7.-14. (canceled)

15. The composition of claim 4 wherein the polymer comprises a polyether.

16. The composition of claim 15 wherein the polyether comprises a residue of polyethylene glycol (PEG) or a copolymer thereof.

17.-23. (canceled)

24. The composition of claim 1 wherein the drug is an anti-cancer agent.

25. The composition of claim 24 wherein the anti-cancer agent is selected from the group consisting of paclitaxel, cisplatin, 5-fluorouracil, doxorubicin, mitoxantrone, etoposide, and derivatives and analogues thereof.

26.-40. (canceled)

41. The composition of claim 1 wherein the microparticle has an average diameter of between about 0.5 mm and about 100 mm.

42. The composition of claim 41 wherein the microparticle has an average diameter of between about 0.5 mm and about 50 mm.

43.-44. (canceled)

45. The composition of claim 1 further comprising a carrier.

46. The composition of claim 45 wherein the carrier comprises a polymer.

47. The composition of claim 45 wherein the carrier is in the form of a gel, hydrogel, paste, ointment, cream, tablet, capsule, spray, powder, film, or surgical sealant.

48.-60. (canceled)

61. A method of treating or preventing a neoplastic disease comprising administering to a patient in need thereof an effective amount of the composition of claim 1, wherein the drug is an anti-neoplastic agent.

62. The method of claim 61 wherein the anti-neoplastic agent is paclitaxel or an analogue or a derivative thereof.

63. The method of claim 61 wherein the neoplastic disease is cancer.

64. A method of treating or preventing fibrosis comprising administering to a patient in need thereof an effective amount of the composition of claim 1, wherein the drug is an anti-fibrotic agent.

65. The method of claim 64 wherein the fibrosis-inhibiting agent is paclitaxel or an analogue or a derivative thereof.

66.-83. (canceled)
